SEQUENCE LISTING (259 sequences)

SEQ ID NO: 1 - protein, 5 aa, synthetic construct
  REGION 1..5: note = synthetic
DYYMN

SEQ ID NO: 2 - protein, 17 aa, synthetic construct
  REGION 1..17: note = synthetic
VINPFNGDTAYKQKFKG

SEQ ID NO: 3 - protein, 11 aa, synthetic construct
  REGION 1..11: note = synthetic
DDGYYLYYFDS

SEQ ID NO: 4 - protein, 11 aa, synthetic construct
  REGION 1..11: note = synthetic
RASQDIGSRLN

SEQ ID NO: 5 - protein, 7 aa, synthetic construct
  REGION 1..7: note = synthetic
ATSSLDS

SEQ ID NO: 6 - protein, 9 aa, synthetic construct
  REGION 1..9: note = synthetic
LQYASSPPT

SEQ ID NO: 7 - protein, 120 aa, synthetic construct
  REGION 1..120: note = synthetic
EVQLQQSGPVLVKPGASVKLSCKASGFTFTDYYMNWVKLSHGKSLEWIGVINPFNGDTAYKQKFKGKATLTVDKSSSTAYMELNSLTSEDSAVYYCARDDGYYLYYFDSWGQGTTLTVSS

SEQ ID NO: 8 - protein, 107 aa, synthetic construct
  REGION 1..107: note = synthetic
DIQMTQSPSSLSASLGERVSLTCRASQDIGSRLNWLQQEPDGTIKRLIYATSSLDSGVPKRFSGSRSGSDYSLTISSLDSEDFVVYYCLQYASSPPTFGAGTKLELK

SEQ ID NO: 9 - protein, 6 aa, synthetic construct
  REGION 1..6: note = synthetic
SGYYWN

SEQ ID NO: 10 - protein, 16 aa, synthetic construct
  REGION 1..16: note = synthetic
DISYDGGNNYNPSLKN

SEQ ID NO: 11 - protein, 10 aa, synthetic construct
  REGION 1..10: note = synthetic
DITLTGAMDY

SEQ ID NO: 12 - protein, 15 aa, synthetic construct
  REGION 1..15: note = synthetic
RASESVDNYGISFMH

SEQ ID NO: 13 - protein, 7 aa, synthetic construct
  REGION 1..7: note = synthetic
RASNLES

SEQ ID NO: 14 - protein, 9 aa, synthetic construct
  REGION 1..9: note = synthetic
QQSNKDPYT

SEQ ID NO: 15 - protein, 119 aa, synthetic construct
  REGION 1..119: note = synthetic
DIQLQESGPGLVKPSQSLSLTCSVTGYSITSGYYWNWIRQFPGNKLEWMGDISYDGGNNYNPSLKNRISITRDTSKNQFFLKLNSVTTEDTATYYCARDITLTGAMDYWGQGTSVTVSS

SEQ ID NO: 16 - protein, 111 aa, synthetic construct
  REGION 1..111: note = synthetic
DIVLTQSPASLAVSLGQRATISCRASESVDNYGISFMHWYQQRPGQPPKLLIYRASNLESGIPARFSGSGSRTDFTLTINPVETDDVATYYCQQSNKDPYTFGGGTKLEIK

SEQ ID NO: 17 - protein, 5 aa, synthetic construct
  REGION 1..5: note = synthetic
ESPMN

SEQ ID NO: 18 - protein, 17 aa, synthetic construct
  REGION 1..17: note = synthetic
MIYTDTGEPTYAEEFKG

SEQ ID NO: 19 - protein, 12 aa, synthetic construct
  REGION 1..12: note = synthetic
DYGSILYWYFDV

SEQ ID NO: 20 - protein, 11 aa, synthetic construct
  REGION 1..11: note = synthetic
KASQDVSTAVA

SEQ ID NO: 21 - protein, 7 aa, synthetic construct
  REGION 1..7: note = synthetic
SASYRYT

SEQ ID NO: 22 - protein, 9 aa, synthetic construct
  REGION 1..9: note = synthetic
QQHYNSPPT

SEQ ID NO: 23 - protein, 121 aa, synthetic construct
  REGION 1..121: note = synthetic
QIQLVQSGPVLKKPGETVKISCKASGYTFTESPMNWVKQAPGRGFKWMGMIYTDTGEPTYAEEFKGRFAFSLETSASTAYLQINNLKSEDTATYFCVSDYGSILYWYFDVWGTGTTVTVSS

SEQ ID NO: 24 - protein, 107 aa, synthetic construct
  REGION 1..107: note = synthetic
DIVMTQSHKFMSTSVGDRVSITCKASQDVSTAVAWYQQTPGQSPKLLIYSASYRYTGVPDRFTGSGSGTDFTFTISSVQAEDLAVYYCQQHYNSPPTFGAGTKLELK

SEQ ID NO: 25 - protein, 17 aa, synthetic construct
  REGION 1..17: note = synthetic
DIDPNNGGTSYNQKFKG

SEQ ID NO: 26 - protein, 16 aa, synthetic construct
  REGION 1..16: note = synthetic
AGSYYYGSSYVEYFDV

SEQ ID NO: 27 - protein, 11 aa, synthetic construct
  REGION 1..11: note = synthetic
RASGNIHNYLA

SEQ ID NO: 28 - protein, 7 aa, synthetic construct
  REGION 1..7: note = synthetic
NAKTLAD

SEQ ID NO: 29 - protein, 9 aa, synthetic construct
  REGION 1..9: note = synthetic
QHFWNTPYT

SEQ ID NO: 30 - protein, 125 aa, synthetic construct
  REGION 1..125: note = synthetic
EVQLQQSGPELVKPGASVKISCKASGYTFTDYYMNWVKQSHGKRLEWIGDIDPNNGGTSYNQKFKGKATLTVDKSSSSAYMDLRSLTSEDSAVYYCARAGSYYYGSSYVEYFDVWGTGTTVTVSS

SEQ ID NO: 31 - protein, 107 aa, synthetic construct
  REGION 1..107: note = synthetic
DIQMTQSPASLSASVGETVTITCRASGNIHNYLAWYQQKQGASPQLLVYNAKTLADGVPSRFSGSGSGTEFSLKINSLQPEDFGNYYCQHFWNTPYTFGGGTKLEIK

SEQ ID NO: 32 - protein, 5 aa, synthetic construct
  REGION 1..5: note = synthetic
RYWMS

SEQ ID NO: 33 - protein, 17 aa, synthetic construct
  REGION 1..17: note = synthetic
EINPDSSTINYAPSLKD

SEQ ID NO: 34 - protein, 20 aa, synthetic construct
  REGION 1..20: note = synthetic
LGIIPTYHYGSSGYYYAMDY

SEQ ID NO: 35 - protein, 16 aa, synthetic construct
  REGION 1..16: note = synthetic
RSSQTIVHGNGHTSLE

SEQ ID NO: 36 - protein, 7 aa, synthetic construct
  REGION 1..7: note = synthetic
KVSNRFS

SEQ ID NO: 37 - protein, 9 aa, synthetic construct
  REGION 1..9: note = synthetic
FQVSHVPYT

SEQ ID NO: 38 - protein, 129 aa, synthetic construct
  REGION 1..129: note = synthetic
EVKILQSGGGLVQPGGSLKLSCAASGIDFSRYWMSWVRRTPGKGLEWIGEINPDSSTINYAPSLKDKFIISRDNAKNTLYLQMSKVRSEDTALYYCARLGIIPTYHYGSSGYYYAMDYWGQGTSVTVSS

SEQ ID NO: 39 - protein, 112 aa, synthetic construct
  REGION 1..112: note = synthetic
DVLMTQTPLSLPVSLGDRASISCRSSQTIVHGNGHTSLEWYLQKPGQSPQLLIYKVSNRFSGVPDRFSASGSGTDFTLTISRVEAEDLGVYYCFQVSHVPYTFGGGTKLEIK

SEQ ID NO: 40 - protein, 5 aa, synthetic construct
  REGION 1..5: note = synthetic
SYAMS

SEQ ID NO: 41 - protein, 17 aa, synthetic construct
  REGION 1..17: note = synthetic
YVTSGGDYIYYADTVKG

SEQ ID NO: 42 - protein, 11 aa, synthetic construct
  REGION 1..11: note = synthetic
VAYDYEWYFDV

SEQ ID NO: 43 - protein, 11 aa, synthetic construct
  REGION 1..11: note = synthetic
KASQDVTTAVA

SEQ ID NO: 44 - protein, 9 aa, synthetic construct
  REGION 1..9: note = synthetic
QQHDSSPWT

SEQ ID NO: 45 - protein, 120 aa, synthetic construct
  REGION 1..120: note = synthetic
DVKLVESGEDLVKPGGSLKLSCAASGFTFSSYAMSWVRQTPEKRLEWVAYVTSGGDYIYYADTVKGRFTISRDDVRNTLYLQMSSLKSEDTAMYYCTRVAYDYEWYFDVWGTGAPVTVSS

SEQ ID NO: 46 - protein, 107 aa, synthetic construct
  REGION 1..107: note = synthetic
DIVMTQSHKFMSTSVGDRVSITCKASQDVTTAVAWYQQKPGQSPKLLIYSASYRYTGVSDRFTGSGSGTDFTFTISSVQAEDLALYYCQQHDSSPWTFGGGTKLEIK

SEQ ID NO: 47 - protein, 5 aa, synthetic construct
  REGION 1..5: note = synthetic
DDYIH

SEQ ID NO: 48 - protein, 17 aa, synthetic construct
  REGION 1..17: note = synthetic
WIDLENGDTEYASKFQG

SEQ ID NO: 49 - protein, 4 aa, synthetic construct
  REGION 1..4: note = synthetic
KGSH

SEQ ID NO: 50 - protein, 16 aa, synthetic construct
  REGION 1..16: note = synthetic
RSSQSLVYSNGNTYLN

SEQ ID NO: 51 - protein, 9 aa, synthetic construct
  REGION 1..9: note = synthetic
SQSTHVPYT

SEQ ID NO: 52 - protein, 113 aa, synthetic construct
  REGION 1..113: note = synthetic
EVQLQQSGAELVRPGASVKLSCTASDFNIKDDYIHWVKQRPEQGLEWIGWIDLENGDTEYASKFQGKATITADTSSNTAYLQLSSLTSEDTAVYYCTTKGSHWGQGTLVTVSA

SEQ ID NO: 53 - protein, 112 aa, synthetic construct
  REGION 1..112: note = synthetic
DVVMTQIPLSLPVSLGDQASISCRSSQSLVYSNGNTYLNWYLQKPGQSPKLLIHKVSNRFSGVPDRFSGSGSGTDFTLKISRVEAEDLGVYFCSQSTHVPYTFGGGTKLEIR

SEQ ID NO: 54 - protein, 5 aa, synthetic construct
  REGION 1..5: note = synthetic
SYGIN

SEQ ID NO: 55 - protein, 17 aa, synthetic construct
  REGION 1..17: note = synthetic
EIYPRSGNTYYNEKFKD

SEQ ID NO: 56 - protein, 13 aa, synthetic construct
  REGION 1..13: note = synthetic
GGTYDGYYYAMDF

SEQ ID NO: 57 - protein, 15 aa, synthetic construct
  REGION 1..15: note = synthetic
RASESVDNFGSSFLH

SEQ ID NO: 58 - protein, 9 aa, synthetic construct
  REGION 1..9: note = synthetic
QQVNKDPRT

SEQ ID NO: 59 - protein, 122 aa, synthetic construct
  REGION 1..122: note = synthetic
QVQLQQSGAELARPGASVKLSCKASGYSFTSYGINWVKQRTGQGLEWIGEIYPRSGNTYYNEKFKDKATLTADKSSSTAYMELRSLTSEDSAVYFCARGGTYDGYYYAMDFWGQGTSVTVSS

SEQ ID NO: 60 - protein, 111 aa, synthetic construct
  REGION 1..111: note = synthetic
DIVLTQSPASLAVSLGQRATISCRASESVDNFGSSFLHWYQQKPGQAPKLLIYRASNLESGIPARFSGSGSRTDFTLTINPVETDDVATYYCQQVNKDPRTFGGGTKLEIK

SEQ ID NO: 61 - protein, 16 aa, synthetic construct
  REGION 1..16: note = synthetic
YISYDGSNEYNLSLKN

SEQ ID NO: 62 - protein, 11 aa, synthetic construct
  REGION 1..11: note = synthetic
EEDGYYGGMDY

SEQ ID NO: 63 - protein, 11 aa, synthetic construct
  REGION 1..11: note = synthetic
RASQDVDTAVA

SEQ ID NO: 64 - protein, 7 aa, synthetic construct
  REGION 1..7: note = synthetic
WASTRHT

SEQ ID NO: 65 - protein, 9 aa, synthetic construct
  REGION 1..9: note = synthetic
QQYSSYPYT

SEQ ID NO: 66 - protein, 120 aa, synthetic construct
  REGION 1..120: note = synthetic
DVQVQESGPGLVKPSQSLSLTCSVTGYSITSGYYWNWIRQFPGNKLEWMGYISYDGSNEYNLSLKNRLSITRDTSKNQFFLKLNSVTTEDTATYYCAREEDGYYGGMDYWGQGTSVTVSS

SEQ ID NO: 67 - protein, 107 aa, synthetic construct
  REGION 1..107: note = synthetic
DIVMTQSHKFMSASVGVRVSITCRASQDVDTAVAWYQQKPGQSPKLLIYWASTRHTGVPDRFTGSGSGTDFTLTISNVQSEDLADYFCQQYSSYPYTFGGGTKLEIK

SEQ ID NO: 68 - protein, 5 aa, synthetic construct
  REGION 1..5: note = synthetic
DYNLN

SEQ ID NO: 69 - protein, 17 aa, synthetic construct
  REGION 1..17: note = synthetic
LVDPIYGTIRYNQNFKD

SEQ ID NO: 70 - protein, 10 aa, synthetic construct
  REGION 1..10: note = synthetic
IMTAVRYFDH

SEQ ID NO: 71 - protein, 16 aa, synthetic construct
  REGION 1..16: note = synthetic
RSSLNIVHSDGNTYLE

SEQ ID NO: 72 - protein, 9 aa, synthetic construct
  REGION 1..9: note = synthetic
FQGSHVPPT

SEQ ID NO: 73 - protein, 119 aa, synthetic construct
  REGION 1..119: note = synthetic
EFQLQQSGPELVKPGASVKISCKASGYSFTDYNLNWVRQSNGKSLEWIGLVDPIYGTIRYNQNFKDKATLTVDQSSSTAYMQLNSLTSEDSAVYYCASIMTAVRYFDHWGQGTTLTVSS

SEQ ID NO: 74 - protein, 112 aa, synthetic construct
  REGION 1..112: note = synthetic
DVLMTQTPLFLPVSLGDQASISCRSSLNIVHSDGNTYLEWYLQKPGQSPKLLIYKVSNRFSGVPDRFSGSGSGTDFTLKISGVEAEDLGVYYCFQGSHVPPTFGGGTKLEIK

SEQ ID NO: 75 - protein, 5 aa, synthetic construct
  REGION 1..5: note = synthetic
SYGVN

SEQ ID NO: 76 - protein, 17 aa, synthetic construct
  REGION 1..17: note = synthetic
EIYPRSGNTYYNERFGD

SEQ ID NO: 77 - protein, 13 aa, synthetic construct
  REGION 1..13: note = synthetic
GGTYDGYYYAMDY

SEQ ID NO: 78 - protein, 15 aa, synthetic construct
  REGION 1..15: note = synthetic
RASESVDNFGSSFMH

SEQ ID NO: 79 - protein, 122 aa, synthetic construct
  REGION 1..122: note = synthetic
QVQLQQSGAELARPGASVKLSCKASGYSFTSYGVNWVKQRTGQGLEWIGEIYPRSGNTYYNERFGDKATLTADKSSSTAYMEIRSLTSEDSAVYFCARGGTYDGYYYAMDYWGQGTSVTVSS

SEQ ID NO: 80 - protein, 111 aa, synthetic construct
  REGION 1..111: note = synthetic
DIVLTQSPASLAVSLGQRATISCRASESVDNFGSSFMHWYQQKPGQAPKLLIYRASNLESGIPARFSGSGSRTDFTLTINPVETDDVATYYCQQVNKDPRTFGGGTKLEIK

SEQ ID NO: 81 - protein, 5 aa, synthetic construct
  REGION 1..5: note = synthetic
DYNMN

SEQ ID NO: 82 - protein, 17 aa, synthetic construct
  REGION 1..17: note = synthetic
LVDPIYGTIRYNQKFKG

SEQ ID NO: 83 - protein, 10 aa, synthetic construct
  REGION 1..10: note = synthetic
ITTSVRYFDY

SEQ ID NO: 84 - protein, 16 aa, synthetic construct
  REGION 1..16: note = synthetic
RSSQNIVHTDGNTYLE

SEQ ID NO: 85 - protein, 119 aa, synthetic construct
  REGION 1..119: note = synthetic
EFQVQQSGPELVKPGASVKISCKASGYSFSDYNMNWLKQSTGKSLEWIGLVDPIYGTIRYNQKFKGKATLTVDQSSSTAYMQLNSLTSEDSAVYYCALITTSVRYFDYWGQGTTLTVSS

SEQ ID NO: 86 - protein, 112 aa, synthetic construct
  REGION 1..112: note = synthetic
DVLMTQTPLSLPVSLGDQASISCRSSQNIVHTDGNTYLEWYLQKPGQSPKLLIYKVSNRFSGVPDRFSGSGSGTDFTLKISRVEAEDLGVYYCFQGSHVPPTFGGGTKLEIK

SEQ ID NO: 87 - protein, 17 aa, synthetic construct
  REGION 1..17: note = synthetic
EIYPRSGNTYYNEKLKG

SEQ ID NO: 88 - protein, 122 aa, synthetic construct
  REGION 1..122: note = synthetic
QVQLQQSGAELARPGASVKLSCKASGFSFTSYGINWVKQRTGQGLEWIGEIYPRSGNTYYNEKLKGEATLTADRSSSTAYMELRSLTSEDSAVYFCARGGTYDGYYYAMDYWGQGTSVTVSS

SEQ ID NO: 89 - protein, 111 aa, synthetic construct
  REGION 1..111: note = synthetic
NIVLTQSPASLAVSLGQRATISCRASESVDNFGSSFMHWYQQKPGQAPKLLIYRASNLESGIPARFGGSGSGTDFTLTIDPVETDDVATYYCQQVNKDPRTFGGGTKLEIK

SEQ ID NO: 90 - protein, 16 aa, synthetic construct
  REGION 1..16: note = synthetic
DISYEGGNNYNPSLKN

SEQ ID NO: 91 - protein, 7 aa, synthetic construct
  REGION 1..7: note = synthetic
RASNLET

SEQ ID NO: 92 - protein, 119 aa, synthetic construct
  REGION 1..119: note = synthetic
QVQLQESGPGLVKPSQTLSLTCTVSGYSISSGYYWNWIRQHPGNKLEWIGDISYEGGNNYNPSLKNRVTISRDTSKNQFSLKLSSVTAADTAVYYCARDITLTGAMDYWGQGTTVTVSS

SEQ ID NO: 93 - protein, 111 aa, synthetic construct
  REGION 1..111: note = synthetic
EIVLTQSPATLSLSPGERATLSCRASESVDNYGISFMHWYQQKPGQAPRLLIYRASNLETGIPARFSGSGSRTDFTLTISSLEPEDFAVYYCQQSNKDPYTFGQGTKLEIK

SEQ ID NO: 94 - protein, 7 aa, synthetic construct
  REGION 1..7: note = synthetic
RASNRES

SEQ ID NO: 95 - protein, 119 aa, synthetic construct
  REGION 1..119: note = synthetic
QVQLQESGPGLVKPSQTLSLTCTVSGYSITSGYYWNWIRQHPGNKLEWIGDISYDGGNNYNPSLKNRVTISRDTSKNQFSLKLSSVTAADTAVYYCARDITLTGAMDYWGQGTTVTVSS

SEQ ID NO: 96 - protein, 111 aa, synthetic construct
  REGION 1..111: note = synthetic
EIVLTQSPATLSLSPGERATLSCRASESVDNYGISFMHWYQQKPGQAPRLLIYRASNRESGIPARFSGSGSRTDFTLTISSLEPEDFAVYYCQQSNKDPYTFGQGTKLEIK

SEQ ID NO: 97 - protein, 6 aa, synthetic construct
  REGION 1..6: note = synthetic
SGYYWT

SEQ ID NO: 98 - protein, 16 aa, synthetic construct
  REGION 1..16: note = synthetic
DISYDAGNNYNPSLKN

SEQ ID NO: 99 - protein, 119 aa, synthetic construct
  REGION 1..119: note = synthetic
QIQLQESGPGLVKPGQTLSLTCTVSGYSITSGYYWTWIRQHPGNKLEWIGDISYDAGNNYNPSLKNRITISRDTSKNQFSLKLSSVTAEDTATYYCARDITLTGAMDYWGQGTTVTVSS

SEQ ID NO: 100 - protein, 111 aa, synthetic construct
  REGION 1..111: note = synthetic
EIVLTQSPATLSLSPGERATLSCRASESVDNYGISFMHWYQQKPGQAPRLLIYRASNLESGIPARFSGSGSGTDFTLTISSLEPEDFAVYYCQQSNKDPYTFGQGTKLEIK

SEQ ID NO: 101 - protein, 120 aa, synthetic construct
  REGION 1..120: note = synthetic
QVQLVESGGGLVKPGGSLRLSCAASGFTFSSYAMSWVRQAPGKRLEWVAYVTSGGDYIYYADTVKGRFTISRDDAKNSLYLQMNSLRAEDTAMYYCTRVAYDYEWYFDVWGQGALVTVSS

SEQ ID NO: 102 - protein, 107 aa, synthetic construct
  REGION 1..107: note = synthetic
DIQMTQSPSSVSTSVGDRVTITCKASQDVTTAVAWYQQKPGKAPKLLIYSASYRYTGVPSRFSGSGSGTDFTFTISSVQPEDFATYYCQQHDSSPWTFGQGTKLEIK

SEQ ID NO: 103 - protein, 120 aa, synthetic construct
  REGION 1..120: note = synthetic
QVQLVESGGGLVKPGGSLRLSCAASGFTFSSYAMSWIRQAPGKGLEWVAYVTSGGDYIYYADTVKGRFTISRDDAKNSLYLQMNSLRAEDTAMYYCTRVAYDYEWYFDVWGQGALVTVSS

SEQ ID NO: 104 - protein, 107 aa, synthetic construct
  REGION 1..107: note = synthetic
DIQMTQSPSSVSASVGDRVTITCKASQDVTTAVAWYQQKPGKAPKLLIYSASYRYTGVPSRFSGSGSGTDFTLTISSLQPEDFATYYCQQHDSSPWTFGQGTKLEIK

SEQ ID NO: 105 - protein, 120 aa, synthetic construct
  REGION 1..120: note = synthetic
QVQLVESGGGLVKPGGSLRLSCAASGFTFSSYAMSWIRQAPGKGLEWVSYVTSGGDYIYYADTVKGRFTISRDDAKNSLYLQMNSLRAEDTAVYYCTRVAYDYEWYFDVWGQGTLVTVSS

SEQ ID NO: 106 - protein, 9 aa, synthetic construct
  REGION 1..9: note = synthetic
QQHDQSPWT

SEQ ID NO: 107 - protein, 107 aa, synthetic construct
  REGION 1..107: note = synthetic
DIQMTQSPSSVSASVGDRVTITCKASQDVTTAVAWYQQKPGKAPKLLIYSASYRYTGVPSRFSGSGSGTDFTLTISSLQPEDFATYYCQQHDQSPWTFGQGTKLEIK

SEQ ID NO: 108 - protein, 17 aa, synthetic construct
  REGION 1..17: note = synthetic
IIYTDTGEPTYAEEFKG

SEQ ID NO: 109 - protein, 9 aa, synthetic construct
  REGION 1..9: note = synthetic
QQHYNAPPT

SEQ ID NO: 110 - protein, 121 aa, synthetic construct
  REGION 1..121: note = synthetic
QVQLVQSGAEVKKPGASVKVSCKASGYTFTESPMNWVRQAPGQGFEWMGIIYTDTGEPTYAEEFKGRVTMTRDTSTSTVYMELSSLRSEDTAVYFCASDYGSILYWYFDVWGQGTLVTVSS

SEQ ID NO: 111 - protein, 107 aa, synthetic construct
  REGION 1..107: note = synthetic
DIQMTQSPSSLSASVGDRVTITCKASQDVSTAVAWYQQKPGKAPKLLIYSASYRYTGVPSRFSGSGSGTDFTLTISSLQPEDFATYYCQQHYNAPPTFGQGTKLEIK

SEQ ID NO: 112 - protein, 107 aa, synthetic construct
  REGION 1..107: note = synthetic
DIQMTQSPSSLSASVGDRVTITCKASQDVSTAVAWYQQKPGKSPKLLIYSASYRYTGVPSRFSGSGSGTDFTFTISSLQPEDFATYYCQQHYNAPPTFGQGTKLEIK

SEQ ID NO: 113 - protein, 107 aa, synthetic construct
  REGION 1..107: note = synthetic
DIQMTQSPSSLSTSVGDRVTITCKASQDVSTAVAWYQQKPGKSPKLLIYSASYRYTGVPSRFSGSGSGTDFTFTISSVQPEDFATYYCQQHYNAPPTFGQGTKLEIK

SEQ ID NO: 114 - protein, 121 aa, synthetic construct
  REGION 1..121: note = synthetic
QVQLVQSGPEVKKPGASVKVSCKASGYTFTESPMNWVRQAPGQGFEWMGIIYTDTGEPTYAEEFKGRVTMTLDTSTSTVYMELSSLRSEDTAVYFCASDYGSILYWYFDVWGQGTLVTVSS

SEQ ID NO: 115 - protein, 121 aa, synthetic construct
  REGION 1..121: note = synthetic
QVQLVQSGPELKKPGASVKVSCKASGYTFTESPMNWVRQAPGQGFEWMGIIYTDTGEPTYAEEFKGRFVFSLDTSVSTAYLQISSLKAEDTAVYFCVSDYGSILYWYFDVWGQGTLVTVSS

SEQ ID NO: 116 - protein, 121 aa, synthetic construct
  REGION 1..121: note = synthetic
QIQLVQSGPELKKPGASVKISCKASGYTFTESPMNWVRQAPGQGFEWMGIIYTDTGEPTYAEEFKGRFVFSLDTSVSTAYLQISSLKAEDTAVYFCVSDYGSILYWYFDVWGQGTLVTVSS

SEQ ID NO: 117 - protein, 30 aa, synthetic construct
  REGION 1..30: note = synthetic
EVQLQQSGPVLVKPGASVKLSCKASGFTFT

SEQ ID NO: 118 - protein, 14 aa, synthetic construct
  REGION 1..14: note = synthetic
WVKLSHGKSLEWIG

SEQ ID NO: 119 - protein, 32 aa, synthetic construct
  REGION 1..32: note = synthetic
KATLTVDKSSSTAYMELNSLTSEDSAVYYCAR

SEQ ID NO: 120 - protein, 11 aa, synthetic construct
  REGION 1..11: note = synthetic
WGQGTTLTVSS

SEQ ID NO: 121 - protein, 23 aa, synthetic construct
  REGION 1..23: note = synthetic
DIQMTQSPSSLSASLGERVSLTC

SEQ ID NO: 122 - protein, 15 aa, synthetic construct
  REGION 1..15: note = synthetic
WLQQEPDGTIKRLIY

SEQ ID NO: 123 - protein, 32 aa, synthetic construct
  REGION 1..32: note = synthetic
GVPKRFSGSRSGSDYSLTISSLDSEDFVVYYC

SEQ ID NO: 124 - protein, 10 aa, synthetic construct
  REGION 1..10: note = synthetic
FGAGTKLELK

SEQ ID NO: 125 - protein, 30 aa, synthetic construct
  REGION 1..30: note = synthetic
DIQLQESGPGLVKPSQSLSLTCSVTGYSIT

SEQ ID NO: 126 - protein, 14 aa, synthetic construct
  REGION 1..14: note = synthetic
WIRQFPGNKLEWMG

SEQ ID NO: 127 - protein, 32 aa, synthetic construct
  REGION 1..32: note = synthetic
RISITRDTSKNQFFLKLNSVTTEDTATYYCAR

SEQ ID NO: 128 - protein, 11 aa, synthetic construct
  REGION 1..11: note = synthetic
WGQGTSVTVSS

SEQ ID NO: 129 - protein, 23 aa, synthetic construct
  REGION 1..23: note = synthetic
DIVLTQSPASLAVSLGQRATISC

SEQ ID NO: 130 - protein, 15 aa, synthetic construct
  REGION 1..15: note = synthetic
WYQQRPGQPPKLLIY

SEQ ID NO: 131 - protein, 32 aa, synthetic construct
  REGION 1..32: note = synthetic
GIPARFSGSGSRTDFTLTINPVETDDVATYYC

SEQ ID NO: 132 - protein, 10 aa, synthetic construct
  REGION 1..10: note = synthetic
FGGGTKLEIK

SEQ ID NO: 133 - protein, 30 aa, synthetic construct
  REGION 1..30: note = synthetic
QIQLVQSGPVLKKPGETVKISCKASGYTFT

SEQ ID NO: 134 - protein, 14 aa, synthetic construct
  REGION 1..14: note = synthetic
WVKQAPGRGFKWMG

SEQ ID NO: 135 - protein, 32 aa, synthetic construct
  REGION 1..32: note = synthetic
RFAFSLETSASTAYLQINNLKSEDTATYFCVS

SEQ ID NO: 136 - protein, 11 aa, synthetic construct
  REGION 1..11: note = synthetic
WGTGTTVTVSS

SEQ ID NO: 137 - protein, 23 aa, synthetic construct
  REGION 1..23: note = synthetic
DIVMTQSHKFMSTSVGDRVSITC

SEQ ID NO: 138 - protein, 15 aa, synthetic construct
  REGION 1..15: note = synthetic
WYQQTPGQSPKLLIY

SEQ ID NO: 139 - protein, 32 aa, synthetic construct
  REGION 1..32: note = synthetic
GVPDRFTGSGSGTDFTFTISSVQAEDLAVYYC

SEQ ID NO: 140 - protein, 30 aa, synthetic construct
  REGION 1..30: note = synthetic
EVQLQQSGPELVKPGASVKISCKASGYTFT

SEQ ID NO: 141 - protein, 14 aa, synthetic construct
  REGION 1..14: note = synthetic
WVKQSHGKRLEWIG

SEQ ID NO: 142 - protein, 32 aa, synthetic construct
  REGION 1..32: note = synthetic
KATLTVDKSSSSAYMDLRSLTSEDSAVYYCAR

SEQ ID NO: 143 - protein, 23 aa, synthetic construct
  REGION 1..23: note = synthetic
DIQMTQSPASLSASVGETVTITC

SEQ ID NO: 144 - protein, 15 aa, synthetic construct
  REGION 1..15: note = synthetic
WYQQKQGASPQLLVY

SEQ ID NO: 145 - protein, 32 aa, synthetic construct
  REGION 1..32: note = synthetic
GVPSRFSGSGSGTEFSLKINSLQPEDFGNYYC

SEQ ID NO: 146 - protein, 30 aa, synthetic construct
  REGION 1..30: note = synthetic
EVKILQSGGGLVQPGGSLKLSCAASGIDFS

SEQ ID NO: 147 - protein, 14 aa, synthetic construct
  REGION 1..14: note = synthetic
WVRRTPGKGLEWIG

SEQ ID NO: 148 - protein, 32 aa, synthetic construct
  REGION 1..32: note = synthetic
KFIISRDNAKNTLYLQMSKVRSEDTALYYCAR

SEQ ID NO: 149 - protein, 23 aa, synthetic construct
  REGION 1..23: note = synthetic
DVLMTQTPLSLPVSLGDRASISC

SEQ ID NO: 150 - protein, 15 aa, synthetic construct
  REGION 1..15: note = synthetic
WYLQKPGQSPQLLIY

SEQ ID NO: 151 - protein, 32 aa, synthetic construct
  REGION 1..32: note = synthetic
GVPDRFSASGSGTDFTLTISRVEAEDLGVYYC

SEQ ID NO: 152 - protein, 30 aa, synthetic construct
  REGION 1..30: note = synthetic
DVKLVESGEDLVKPGGSLKLSCAASGFTFS

SEQ ID NO: 153 - protein, 14 aa, synthetic construct
  REGION 1..14: note = synthetic
WVRQTPEKRLEWVA

SEQ ID NO: 154 - protein, 32 aa, synthetic construct
  REGION 1..32: note = synthetic
RFTISRDDVRNTLYLQMSSLKSEDTAMYYCTR

SEQ ID NO: 155 - protein, 11 aa, synthetic construct
  REGION 1..11: note = synthetic
WGTGAPVTVSS

SEQ ID NO: 156 - protein, 15 aa, synthetic construct
  REGION 1..15: note = synthetic
WYQQKPGQSPKLLIY

SEQ ID NO: 157 - protein, 32 aa, synthetic construct
  REGION 1..32: note = synthetic
GVSDRFTGSGSGTDFTFTISSVQAEDLALYYC

SEQ ID NO: 158 - protein, 30 aa, synthetic construct
  REGION 1..30: note = synthetic
EVQLQQSGAELVRPGASVKLSCTASDFNIK

SEQ ID NO: 159 - protein, 14 aa, synthetic construct
  REGION 1..14: note = synthetic
WVKQRPEQGLEWIG

SEQ ID NO: 160 - protein, 32 aa, synthetic construct
  REGION 1..32: note = synthetic
KATITADTSSNTAYLQLSSLTSEDTAVYYCTT

SEQ ID NO: 161 - protein, 11 aa, synthetic construct
  REGION 1..11: note = synthetic
WGQGTLVTVSA

SEQ ID NO: 162 - protein, 23 aa, synthetic construct
  REGION 1..23: note = synthetic
DVVMTQIPLSLPVSLGDQASISC

SEQ ID NO: 163 - protein, 15 aa, synthetic construct
  REGION 1..15: note = synthetic
WYLQKPGQSPKLLIH

SEQ ID NO: 164 - protein, 32 aa, synthetic construct
  REGION 1..32: note = synthetic
GVPDRFSGSGSGTDFTLKISRVEAEDLGVYFC

SEQ ID NO: 165 - protein, 10 aa, synthetic construct
  REGION 1..10: note = synthetic
FGGGTKLEIR

SEQ ID NO: 166 - protein, 30 aa, synthetic construct
  REGION 1..30: note = synthetic
QVQLQQSGAELARPGASVKLSCKASGYSFT

SEQ ID NO: 167 - protein, 14 aa, synthetic construct
  REGION 1..14: note = synthetic
WVKQRTGQGLEWIG

SEQ ID NO: 168 - protein, 32 aa, synthetic construct
  REGION 1..32: note = synthetic
KATLTADKSSSTAYMELRSLTSEDSAVYFCAR

SEQ ID NO: 169 - protein, 15 aa, synthetic construct
  REGION 1..15: note = synthetic
WYQQKPGQAPKLLIY

SEQ ID NO: 170 - protein, 30 aa, synthetic construct
  REGION 1..30: note = synthetic
DVQVQESGPGLVKPSQSLSLTCSVTGYSIT

SEQ ID NO: 171 - protein, 32 aa, synthetic construct
  REGION 1..32: note = synthetic
RLSITRDTSKNQFFLKLNSVTTEDTATYYCAR

SEQ ID NO: 172 - protein, 23 aa, synthetic construct
  REGION 1..23: note = synthetic
DIVMTQSHKFMSASVGVRVSITC

SEQ ID NO: 173 - protein, 32 aa, synthetic construct
  REGION 1..32: note = synthetic
GVPDRFTGSGSGTDFTLTISNVQSEDLADYFC

SEQ ID NO: 174 - protein, 30 aa, synthetic construct
  REGION 1..30: note = synthetic
EFQLQQSGPELVKPGASVKISCKASGYSFT

SEQ ID NO: 175 - protein, 14 aa, synthetic construct
  REGION 1..14: note = synthetic
WVRQSNGKSLEWIG

SEQ ID NO: 176 - protein, 32 aa, synthetic construct
  REGION 1..32: note = synthetic
KATLTVDQSSSTAYMQLNSLTSEDSAVYYCAS

SEQ ID NO: 177 - protein, 23 aa, synthetic construct
  REGION 1..23: note = synthetic
DVLMTQTPLFLPVSLGDQASISC

SEQ ID NO: 178 - protein, 15 aa, synthetic construct
  REGION 1..15: note = synthetic
WYLQKPGQSPKLLIY

SEQ ID NO: 179 - protein, 32 aa, synthetic construct
  REGION 1..32: note = synthetic
GVPDRFSGSGSGTDFTLKISGVEAEDLGVYYC

SEQ ID NO: 180 - protein, 30 aa, synthetic construct
  REGION 1..30: note = synthetic
EFQVQQSGPELVKPGASVKISCKASGYSFS

SEQ ID NO: 181 - protein, 14 aa, synthetic construct
  REGION 1..14: note = synthetic
WLKQSTGKSLEWIG

SEQ ID NO: 182 - protein, 32 aa, synthetic construct
  REGION 1..32: note = synthetic
KATLTVDQSSSTAYMQLNSLTSEDSAVYYCAL

SEQ ID NO: 183 - protein, 23 aa, synthetic construct
  REGION 1..23: note = synthetic
DVLMTQTPLSLPVSLGDQASISC

SEQ ID NO: 184 - protein, 32 aa, synthetic construct
  REGION 1..32: note = synthetic
GVPDRFSGSGSGTDFTLKISRVEAEDLGVYYC

SEQ ID NO: 185 - protein, 30 aa, synthetic construct
  REGION 1..30: note = synthetic
QVQLQQSGAELARPGASVKLSCKASGFSFT

SEQ ID NO: 186 - protein, 32 aa, synthetic construct
  REGION 1..32: note = synthetic
EATLTADRSSSTAYMELRSLTSEDSAVYFCAR

SEQ ID NO: 187 - protein, 23 aa, synthetic construct
  REGION 1..23: note = synthetic
NIVLTQSPASLAVSLGQRATISC

SEQ ID NO: 188 - protein, 39 aa, synthetic construct
  REGION 1..39: note = synthetic
RASNLESGIPARFGGSGSGTDFTLTIDPVETDDVATYYC

SEQ ID NO: 189 - protein, 30 aa, synthetic construct
  REGION 1..30: note = synthetic
QVQLQESGPGLVKPSQTLSLTCTVSGYSIS

SEQ ID NO: 190 - protein, 14 aa, synthetic construct
  REGION 1..14: note = synthetic
WIRQHPGNKLEWIG

SEQ ID NO: 191 - protein, 32 aa, synthetic construct
  REGION 1..32: note = synthetic
RVTISRDTSKNQFSLKLSSVTAADTAVYYCAR

SEQ ID NO: 192 - protein, 11 aa, synthetic construct
  REGION 1..11: note = synthetic
WGQGTTVTVSS

SEQ ID NO: 193 - protein, 23 aa, synthetic construct
  REGION 1..23: note = synthetic
EIVLTQSPATLSLSPGERATLSC

SEQ ID NO: 194 - protein, 15 aa, synthetic construct
  REGION 1..15: note = synthetic
WYQQKPGQAPRLLIY

SEQ ID NO: 195 - protein, 32 aa, synthetic construct
  REGION 1..32: note = synthetic
GIPARFSGSGSRTDFTLTISSLEPEDFAVYYC

SEQ ID NO: 196 - protein, 10 aa, synthetic construct
  REGION 1..10: note = synthetic
FGQGTKLEIK

SEQ ID NO: 197 - protein, 30 aa, synthetic construct
  REGION 1..30: note = synthetic
QVQLQESGPGLVKPSQTLSLTCTVSGYSIT

SEQ ID NO: 198 - protein, 30 aa, synthetic construct
  REGION 1..30: note = synthetic
QIQLQESGPGLVKPGQTLSLTCTVSGYSIT

SEQ ID NO: 199 - protein, 32 aa, synthetic construct
  REGION 1..32: note = synthetic
RITISRDTSKNQFSLKLSSVTAEDTATYYCAR

SEQ ID NO: 200 - protein, 32 aa, synthetic construct
  REGION 1..32: note = synthetic
GIPARFSGSGSGTDFTLTISSLEPEDFAVYYC

SEQ ID NO: 201 - protein, 30 aa, synthetic construct
  REGION 1..30: note = synthetic
QVQLVESGGGLVKPGGSLRLSCAASGFTFS

SEQ ID NO: 202 - protein, 14 aa, synthetic construct
  REGION 1..14: note = synthetic
WVRQAPGKRLEWVA

SEQ ID NO: 203 - protein, 32 aa, synthetic construct
  REGION 1..32: note = synthetic
RFTISRDDAKNSLYLQMNSLRAEDTAMYYCTR

SEQ ID NO: 204 - protein, 11 aa, synthetic construct
  REGION 1..11: note = synthetic
WGQGALVTVSS

SEQ ID NO: 205 - protein, 23 aa, synthetic construct
  REGION 1..23: note = synthetic
DIQMTQSPSSVSTSVGDRVTITC

SEQ ID NO: 206 - protein, 15 aa, synthetic construct
  REGION 1..15: note = synthetic
WYQQKPGKAPKLLIY

SEQ ID NO: 207 - protein, 32 aa, synthetic construct
  REGION 1..32: note = synthetic
GVPSRFSGSGSGTDFTFTISSVQPEDFATYYC

SEQ ID NO: 208 - protein, 14 aa, synthetic construct
  REGION 1..14: note = synthetic
WIRQAPGKGLEWVA

SEQ ID NO: 209 - protein, 23 aa, synthetic construct
  REGION 1..23: note = synthetic
DIQMTQSPSSVSASVGDRVTITC

SEQ ID NO: 210 - protein, 32 aa, synthetic construct
  REGION 1..32: note = synthetic
GVPSRFSGSGSGTDFTLTISSLQPEDFATYYC

SEQ ID NO: 211 - protein, 14 aa, synthetic construct
  REGION 1..14: note = synthetic
WIRQAPGKGLEWVS

SEQ ID NO: 212 - protein, 32 aa, synthetic construct
  REGION 1..32: note = synthetic
RFTISRDDAKNSLYLQMNSLRAEDTAVYYCTR

SEQ ID NO: 213 - protein, 11 aa, synthetic construct
  REGION 1..11: note = synthetic
WGQGTLVTVSS

SEQ ID NO: 214 - protein, 30 aa, synthetic construct
  REGION 1..30: note = synthetic
QVQLVQSGAEVKKPGASVKVSCKASGYTFT

SEQ ID NO: 215 - protein, 14 aa, synthetic construct
  REGION 1..14: note = synthetic
WVRQAPGQGFEWMG

SEQ ID NO: 216 - protein, 32 aa, synthetic construct
  REGION 1..32: note = synthetic
RVTMTRDTSTSTVYMELSSLRSEDTAVYFCAS

SEQ ID NO: 217 - protein, 23 aa, synthetic construct
  REGION 1..23: note = synthetic
DIQMTQSPSSLSASVGDRVTITC

SEQ ID NO: 218 - protein, 15 aa, synthetic construct
  REGION 1..15: note = synthetic
WYQQKPGKSPKLLIY

SEQ ID NO: 219 - protein, 32 aa, synthetic construct
  REGION 1..32: note = synthetic
GVPSRFSGSGSGTDFTFTISSLQPEDFATYYC

SEQ ID NO: 220 - protein, 23 aa, synthetic construct
  REGION 1..23: note = synthetic
DIQMTQSPSSLSTSVGDRVTITC

SEQ ID NO: 221 - protein, 30 aa, synthetic construct
  REGION 1..30: note = synthetic
QVQLVQSGPEVKKPGASVKVSCKASGYTFT

SEQ ID NO: 222 - protein, 32 aa, synthetic construct
  REGION 1..32: note = synthetic
RVTMTLDTSTSTVYMELSSLRSEDTAVYFCAS

SEQ ID NO: 223 - protein, 30 aa, synthetic construct
  REGION 1..30: note = synthetic
QVQLVQSGPELKKPGASVKVSCKASGYTFT

SEQ ID NO: 224 - protein, 32 aa, synthetic construct
  REGION 1..32: note = synthetic
RFVFSLDTSVSTAYLQISSLKAEDTAVYFCVS

SEQ ID NO: 225 - protein, 30 aa, synthetic construct
  REGION 1..30: note = synthetic
QIQLVQSGPELKKPGASVKISCKASGYTFT

SEQ ID NO: 226 - protein, 5 aa, synthetic construct
  REGION 1..5: note = synthetic
  VAR_SEQ 4: note = X can be I or V
SYGXN

SEQ ID NO: 227 - protein, 17 aa, synthetic construct
  REGION 1..17: note = synthetic
  VAR_SEQ 14: note = X can be K or R
  VAR_SEQ 15: note = X can be F or L
  VAR_SEQ 16: note = X can be K or G
  VAR_SEQ 17: note = X can be D or G
EIYPRSGNTYYNEXXXX

SEQ ID NO: 228 - protein, 13 aa, synthetic construct
  REGION 1..13: note = synthetic
  VAR_SEQ 13: note = X can be F or Y
GGTYDGYYYAMDX

SEQ ID NO: 229 - protein, 15 aa, synthetic construct
  REGION 1..15: note = synthetic
  VAR_SEQ 14: note = X can be L or M
RASESVDNFGSSFXH

SEQ ID NO: 230 - protein, 5 aa, synthetic construct
  REGION 1..5: note = synthetic
  VAR_SEQ 4: note = X can be L or M
DYNXN

SEQ ID NO: 231 - protein, 17 aa, synthetic construct
  REGION 1..17: note = synthetic
  VAR_SEQ 14: note = X can be N or K
  VAR_SEQ 17: note = X can be D or G
LVDPIYGTIRYNQXFKX

SEQ ID NO: 232 - protein, 10 aa, synthetic construct
  REGION 1..10: note = synthetic
  VAR_SEQ 2: note = X can be M or T
  VAR_SEQ 4: note = X can be A or S
  VAR_SEQ 10: note = X can be H or Y
IXTXVRYFDX

SEQ ID NO: 233 - protein, 16 aa, synthetic construct
  REGION 1..16: note = synthetic
  VAR_SEQ 4: note = X can be L or Q
  VAR_SEQ 9: note = X can be S or T
RSSXNIVHXDGNTYLE

SEQ ID NO: 234 - protein, 6 aa, synthetic construct
  REGION 1..6: note = synthetic
  VAR_SEQ 6: note = X can be N or T
SGYYWX

SEQ ID NO: 235 - protein, 16 aa, synthetic construct
  REGION 1..16: note = synthetic
  VAR_SEQ 5: note = X can be E or D
  VAR_SEQ 6: note = X can be G or A
DISYXXGNNYNPSLKN

SEQ ID NO: 236 - protein, 7 aa, synthetic construct
  REGION 1..7: note = synthetic
  VAR_SEQ 5: note = X can be L or R
  VAR_SEQ 7: note = X can be T or S
RASNXEX

SEQ ID NO: 237 - protein, 9 aa, synthetic construct
  REGION 1..9: note = synthetic
  VAR_SEQ 5: note = X can be S or Q
QQHDXSPWT

SEQ ID NO: 238 - protein, 17 aa, synthetic construct
  REGION 1..17: note = synthetic
  VAR_SEQ 1: note = X can be M or I
XIYTDTGEPTYAEEFKG

SEQ ID NO: 239 - protein, 9 aa, synthetic construct
  REGION 1..9: note = synthetic
  VAR_SEQ 6: note = X can be A or S
QQHYNXPPT

SEQ ID NO: 240 - protein, 30 aa, synthetic construct
  REGION 1..30: note = synthetic
  VAR_SEQ 2: note = X can be V or I
  VAR_SEQ 15: note = X can be S or G
  VAR_SEQ 30: note = X can be S or T
QXQLQESGPGLVKPXQTLSLTCTVSGYSIX

SEQ ID NO: 241 - protein, 32 aa, synthetic construct
  REGION 1..32: note = synthetic
  VAR_SEQ 2: note = X can be V or I
  VAR_SEQ 23: note = X can be A or E
  VAR_SEQ 27: note = X can be V or T
RXTISRDTSKNQFSLKLSSVTAXDTAXYYCAR

SEQ ID NO: 242 - protein, 32 aa, synthetic construct
  REGION 1..32: note = synthetic
  VAR_SEQ 12: note = X can be R or G
GIPARFSGSGSXTDFTLTISSLEPEDFAVYYC

SEQ ID NO: 243 - protein, 14 aa, synthetic construct
  REGION 1..14: note = synthetic
  VAR_SEQ 2: note = X can be V or I
  VAR_SEQ 9: note = X can be R or G
  VAR_SEQ 14: note = X can be A or S
WXRQAPGKXLEWVX

SEQ ID NO: 244 - protein, 32 aa, synthetic construct
  REGION 1..32: note = synthetic
  VAR_SEQ 27: note = X can be M or V
RFTISRDDAKNSLYLQMNSLRAEDTAXYYCTR

SEQ ID NO: 245 - protein, 11 aa, synthetic construct
  REGION 1..11: note = synthetic
  VAR_SEQ 5: note = X can be A or T
WGQGXLVTVSS

SEQ ID NO: 246 - protein, 23 aa, synthetic construct
  REGION 1..23: note = synthetic
  VAR_SEQ 13: note = X can be T or A
DIQMTQSPSSVSXSVGDRVTITC

SEQ ID NO: 247 - protein, 32 aa, synthetic construct
  REGION 1..32: note = synthetic
  VAR_SEQ 17: note = X can be L or F
  VAR_SEQ 22: note = X can be L or V
GVPSRFSGSGSGTDFTXTISSXQPEDFATYYC

SEQ ID NO: 248 - protein, 30 aa, synthetic construct
  REGION 1..30: note = synthetic
  VAR_SEQ 2: note = X can be V or I
  VAR_SEQ 9: note = X can be A or P
  VAR_SEQ 11: note = X can be V or L
  VAR_SEQ 20: note = X can be V or I
QXQLVQSGXEXKKPGASVKXSCKASGYTFT

SEQ ID NO: 249 - protein, 32 aa, synthetic construct
  REGION 1..32: note = synthetic
  VAR_SEQ 2: note = X can be V or F
  VAR_SEQ 3: note = X can be T or V
  VAR_SEQ 4: note = X can be M or F
  VAR_SEQ 5: note = X can be T or S
  VAR_SEQ 6: note = X can be R or L
  VAR_SEQ 10: note = X can be T or V
  VAR_SEQ 13: note = X can be V or A
  VAR_SEQ 15: note = X can be M or L
  VAR_SEQ 16: note = X can be E or Q
  VAR_SEQ 17: note = X can be L or I
  VAR_SEQ 21: note = X can be R or K
  VAR_SEQ 22: note = X can be S or A
  VAR_SEQ 31: note = X can be A or V
RXXXXXDTSXSTXYXXXSSLXXEDTAVYFCXS

SEQ ID NO: 250 - protein, 23 aa, synthetic construct
  REGION 1..23: note = synthetic
  VAR_SEQ 13: note = X can be T or A
DIQMTQSPSSLSXSVGDRVTITC

SEQ ID NO: 251 - protein, 15 aa, synthetic construct
  REGION 1..15: note = synthetic
  VAR_SEQ 9: note = X can be A or S
WYQQKPGKXPKLLIY

SEQ ID NO: 252 - protein, 32 aa, synthetic construct; identical to SEQ ID NO: 247
  REGION 1..32: note = synthetic
  VAR_SEQ 17: note = X can be L or F
  VAR_SEQ 22: note = X can be L or V

SEQ ID NO: 253 - protein, 120 aa, synthetic construct
  REGION 1..120: note = synthetic
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 254 - protein, 107 aa, synthetic construct
  REGION 1..107: note = synthetic
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 255 - protein, 125 aa, synthetic construct
  REGION 1..125: note = synthetic
QIQLVQSGPELKKPGETVKISCKASGFTLTNYGMNWVRQTPGKGLKWMGWINTDTGEPTYADDFKGRFAFSLETSASTASLQINNLKNADTATYFCARNPPYYYGTNNAEAMDYWGQGTAVTVSS

SEQ ID NO: 256 - protein, 107 aa, synthetic construct
  REGION 1..107: note = synthetic
DIQMTQTTSSLSASLGDRVTISCSSSQDISNYLNWYQQKPDGTVKVLIYYTSTLHLGVPSRFSGSGSGTDYSLTISNLELEDIATYYCQQYYNLPWTFGGGTKLEIK

SEQ ID NO: 257 - protein, 114 aa, synthetic construct
  REGION 1..114: note = synthetic
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSS

SEQ ID NO: 258 - protein, 112 aa, synthetic construct
  REGION 1..112: note = synthetic
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIK

SEQ ID NO: 259 - protein, 327 aa, synthetic construct
  REGION 1..327: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0313625 A1
(43) Pub. Date: Oct. 9, 2025

(54) NOVEL ANTI-LAG3 ANTIBODIES

(71) Applicants: ELPISCIENCE (SUZHOU) BIOPHARMA, LTD., Suzhou, Jiangsu (CN); ELPISCIENCE BIOPHARMA, LTD., Shanghai (CN)

(72) Inventors: Xiaofeng NIU, Shanghai (CN); Jinfeng ZHAO, Shanghai (CN); Roumei XING, Shanghai (CN); Zhihao WU, Shanghai (CN); Rui GAO, Shanghai (CN); Yangsheng QIU, Shanghai (CN); Hongtao LU, Shanghai (CN)

(21) Appl. No.: 18/704,936

(22) PCT Filed: Nov. 1, 2022
(86) PCT No.: PCT/CN2022/128858

§ 371 Date: Apr. 26, 2024

(30) Foreign Application Priority Data
Nov. 1, 2021 (WO) PCT/CN2021/127989
Oct. 10, 2022 (WO) PCT/CN2022/124457

(51) Int. Cl. C07K 16/28; A61K 39/00; A61K 47/68; A61P 35/00

(52) U.S. Cl. CPC C07K 16/2803; A61K 47/6849; A61P 35/00; A61K 2039/505; C07K 2317/24; C07K 2317/52; C07K 2317/565; C07K 2317/92

(57) ABSTRACT

Provided are anti-LAG3 antibodies or antigen-binding fragments thereof, isolated polynucleotides encoding the same, pharmaceutical compositions comprising the same and the uses thereof.

### FIELD OF THE INVENTION

The present disclosure generally relates to novel anti-LAG3 antibodies.

### BACKGROUND

Lymphocyte activation gene 3 (LAG-3) belongs to the immunoglobulin superfamily, which consists of an extracellular region, a transmembrane region and a cytoplasmic region. The gene of LAG-3 is located on chromosome 12P13, which is similar to the location and structure of CD4 gene on chromosome. LAG-3 is expressed on activated T cells, exhausted T cells, tumor infiltrating T cells and regulatory T cells (Treg).

LAG3 is a multiple ligands blocker, whose ligands include MHC II, FGL1, LSECtin, etc. LAG3's main ligand is MHC class II, to which it binds with higher affinity than CD4. The protein negatively regulates cellular proliferation, activation, and homeostasis of T cells, in a similar fashion to CTLA-4 and PD-1 and has been reported to play a role in Treg suppressive function. FGL1 (Fibrinogen-like protein1), a liver-secreted protein, is another (major) LAG3 functional ligand independent of MHC-II. Manipulating FGL-1 binding to T cells has been proposed for both cancer immunotherapy and anti-inflammatory treatments. LSECtin is also a ligand of LAG3, which can interact with glycans on LAG3. LSECtin belongs to C-type lectin and is mainly expressed in liver. In addition, a variety of tumor cells express LSECtin, including melanoma, bladder cancer and pancreatic cancer.

Meanwhile, inhibition of LAG-3 can also reduce the inhibition of regulatory T cells on immune response. Therefore, LAG-3 is considered to be a more attractive target than other immune checkpoint proteins. Antibody drugs targeting LAG-3 may become important antitumor drugs in the future. However, at present, very few drugs in the world have been approved on the market targeting LAG-3.

There is still a lack of high-affinity antibodies targeting LAG-3 with good activity to block the interaction of LAG-3 with multiple ligands, as well as better efficacy and pharmacokinetics (PK).

Needs remain for novel anti-LAG-3 antibodies.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides an antibody or an antigen-binding fragment thereof capable of specifically binding to LAG3, comprising a heavy chain variable region comprising HCDR1, HCDR2 and HCDR3, and/or a light chain variable region comprising LCDR1, LCDR2 and LCDR3, wherein the HCDR1 comprises an amino acid sequence of SYGX₁N (SEQ ID NO: 226), the HCDR2 comprises an amino acid sequence of EIYPRSGNTYYNEX₂X₃X₄X₅(SEQ ID NO: 227), the HCDR3 comprises an amino acid sequence of GGTYDGYYYAMDX₆ (SEQ ID NO: 228), the LCDR1 comprises an amino acid sequence of RASESVDNFGSSFX₇H (SEQ ID NO: 229), the LCDR2 comprises an amino acid sequence of SEQ ID NO: 13, and the LCDR3 comprises an amino acid sequence of SEQ ID NO: 58, wherein, X₁ is I or V, X₂ is K or R, X₃ is F or L, X₄ is K or G, X₅ is D or G, X₆ is F or Y, X₇ is L or M; or the HCDR1 comprises an amino acid sequence of DYNX₈N (SEQ ID NO: 230), the HCDR2 comprises an amino acid sequence of LVDPIYGTIRYNQX₉FKX₁₀ (SEQ ID NO: 231), the HCDR3 comprises an amino acid sequence of IX₁₁TX₁₂VRYFDX₁₃ (SEQ ID NO: 232), the LCDR1 comprises an amino acid sequence of RSSX₁₄NIVHX₁₅DGNTYLE (SEQ ID NO: 233), the LCDR2 comprises an amino acid sequence of SEQ ID NO: 36, and the LCDR3 comprises an amino acid sequence of SEQ ID NO: 72, wherein, X₈ is L or M, X₉ is N or K, X₁₀ is D or G, X₁ is M or T, X₁₂ is A or S, X₁₃ is H or Y, X₁₄ is L or Q, X₁₅ is S or T; or the HCDR1 comprises an amino acid sequence of SGYYWX₁₆ (SEQ ID NO: 234), the HCDR2 comprises an amino acid sequence of DISYX₁₇X₁₈GNNYNPSLKN (SEQ ID NO: 235), the HCDR3 comprises an amino acid sequence of SEQ ID NO: 11, the LCDR1 comprises an amino acid sequence of SEQ ID NO: 12, the LCDR2 comprises an amino acid sequence of RASNX₁₉EX₂₀ (SEQ ID NO: 236), and the LCDR3 comprises an amino acid sequence of SEQ ID NO: 14, wherein, X₁₆ is N or T, X₁₇ is E or D, X₁₈ is G or A, X₁₉ is L or R, X₂₀ is T or S; or the HCDR1 comprises an amino acid sequence of SEQ ID NO: 40, the HCDR2 comprises an amino acid sequence of SEQ ID NO: 41, the HCDR3 comprises an amino acid sequence of SEQ ID NO: 42, the LCDR1 comprises an amino acid sequence of SEQ ID NO: 43, the LCDR2 comprises an amino acid sequence of SEQ ID NO: 21, and the LCDR3 comprises an amino acid sequence of QQHDX₂₁SPWT (SEQ ID NO: 237) wherein, X₂₁ is S or Q; or the HCDR1 comprises an amino acid sequence of SEQ ID NO: 17, the HCDR2 comprises an amino acid sequence of X₂₂IYTDTGEPTYAEEFKG (SEQ ID NO: 238); the HCDR3 comprises an amino acid sequence of SEQ ID NO: 19, the LCDR1 comprises an amino acid sequence of SEQ ID NO: 20, the LCDR2 comprises an amino acid sequence of SEQ ID NO: 21, and the LCDR3 comprises an amino acid sequence of QQHYNX₂₃PPT (SEQ ID NO: 239) wherein, X₂₂ is M or I, X₂₃ is A or S; or the HCDR1 comprises an amino acid sequence of SEQ ID NO: 1, the HCDR2 comprises an amino acid sequence of SEQ ID NO: 2, the HCDR3 comprises an amino acid sequence of SEQ ID NO: 3, the LCDR1 comprises an amino acid sequence of SEQ ID NO: 4, the LCDR2 comprises an amino acid sequence of SEQ ID NO: 5, and the LCDR3 comprises an amino acid sequence of SEQ ID NO: 6, or the HCDR1 comprises an amino acid sequence of SEQ ID NO: 1, the HCDR2 comprises an amino acid sequence of SEQ ID NO: 25, the HCDR3 comprises an amino acid sequence of SEQ ID NO: 26, the LCDR1 comprises an amino acid sequence of SEQ ID NO: 27, the LCDR2 comprises an amino acid sequence of SEQ ID NO: 28, and the LCDR3 comprises an amino acid sequence of SEQ ID NO: 29, or the HCDR1 comprises an amino acid sequence of SEQ ID NO: 32, the HCDR2 comprises an amino acid sequence of SEQ ID NO: 33, the HCDR3 comprises an amino acid sequence of SEQ ID NO: 34, the LCDR1 comprises an amino acid sequence of SEQ ID NO: 35, the LCDR2 comprises an amino acid sequence of SEQ ID NO: 36, and the LCDR3 comprises an amino acid sequence of SEQ ID NO: 37, or the HCDR1 comprises an amino acid sequence of SEQ ID NO: 47, the HCDR2 comprises an amino acid sequence of SEQ ID NO: 48, the HCDR3 comprises an amino acid sequence of SEQ ID NO: 49, the LCDR1 comprises an amino acid sequence of SEQ ID NO: 50, the LCDR2 comprises an amino acid sequence of SEQ ID NO: 36, and the LCDR3 comprises an amino acid sequence of SEQ ID NO: 51, or the HCDR1 comprises an amino acid sequence of SEQ ID NO: 9, the HCDR2 comprises an amino acid sequence of SEQ ID NO: 61, the HCDR3 comprises an amino acid sequence of SEQ ID NO: 62, the LCDR1 comprises an amino acid sequence of SEQ ID NO: 63, the LCDR2 comprises an amino acid sequence of SEQ ID NO: 64, and the LCDR3 comprises an amino acid sequence of SEQ ID NO: 65.

In some embodiments, the HCDR1 comprises an amino acid sequence of SEQ ID NO: 54 or 75, the HCDR2 comprises an amino acid sequence of SEQ ID NO: 55, 76 or 87, the HCDR3 comprises an amino acid sequence of SEQ ID NO: 56 or 77, the LCDR1 comprises an amino acid sequence of SEQ ID NO: 57 or 78, the LCDR2 comprises an amino acid sequence of SEQ ID NO: 13, and the LCDR3 comprises an amino acid sequence of SEQ ID NO: 58.

In some embodiments, the HCDR1 comprises an amino acid sequence of SEQ ID NO: 68 or 81, the HCDR2 comprises an amino acid sequence of SEQ ID NO: 69 or 82, the HCDR3 comprises an amino acid sequence of SEQ ID NO: 70 or 83, the LCDR1 comprises an amino acid sequence of SEQ ID NO: 71 or 84, the LCDR2 comprises an amino acid sequence of SEQ ID NO: 36, and the LCDR3 comprises an amino acid sequence of SEQ ID NO: 72.

In some embodiments, the HCDR1 comprises an amino acid sequence of SEQ ID NO: 9 or 97, the HCDR2 comprises an amino acid sequence of SEQ ID NO: 90, 10 or 98, the HCDR3 comprises an amino acid sequence of SEQ ID NO: 11, the LCDR1 comprises an amino acid sequence of SEQ ID NO: 12, the LCDR2 comprises an amino acid sequence of SEQ ID NO: 91, 94, or 13, and the LCDR3 comprises an amino acid sequence of SEQ ID NO: 14.

In some embodiments, the HCDR1 comprises an amino acid sequence of SEQ ID NO: 40, the HCDR2 comprises an amino acid sequence of SEQ ID NO: 41, the HCDR3 comprises an amino acid sequence of SEQ ID NO: 42, the LCDR1 comprises an amino acid sequence of SEQ ID NO: 43, the LCDR2 comprises an amino acid sequence of SEQ ID NO: 21, and the LCDR3 comprises an amino acid sequence of SEQ ID NO: 44 or 106.

In some embodiments, the HCDR1 comprises an amino acid sequence of SEQ ID NO: 17, the HCDR2 comprises an amino acid sequence of SEQ ID NO: 18 or 108, the HCDR3 comprises an amino acid sequence of SEQ ID NO: 19, the LCDR1 comprises an amino acid sequence of SEQ ID NO: 20, the LCDR2 comprises an amino acid sequence of SEQ ID NO: 21, and the LCDR3 comprises an amino acid sequence of SEQ ID NO: 22 or 109.

In some embodiments, the HCDR1 comprises an amino acid sequence of SEQ ID NO: 54; the HCDR2 comprises an amino acid sequence of SEQ ID NO: 55; the HCDR3 comprises an amino acid sequence of SEQ ID NO: 56; the LCDR1 comprises an amino acid sequence of SEQ ID NO: 57; the LCDR2 comprises an amino acid sequence of SEQ ID NO: 13; and the LCDR3 comprises an amino acid sequence of SEQ ID NO: 58; or the HCDR1 comprises an amino acid sequence of SEQ ID NO: 75; the HCDR2 comprises an amino acid sequence of SEQ ID NO: 76; the HCDR3 comprises an amino acid sequence of SEQ ID NO: 77; the LCDR1 comprises an amino acid sequence of SEQ ID NO: 78; the LCDR2 comprises an amino acid sequence of SEQ ID NO: 13; and the LCDR3 comprises an amino acid sequence of SEQ ID NO: 58; or the HCDR1 comprises an amino acid sequence of SEQ ID NO: 54; the HCDR2 comprises an amino acid sequence of SEQ ID NO: 87; the HCDR3 comprises an amino acid sequence of SEQ ID NO: 77; the LCDR1 comprises an amino acid sequence of SEQ ID NO: 78; the LCDR2 comprises an amino acid sequence of SEQ ID NO: 13; and the LCDR3 comprises an amino acid sequence of SEQ ID NO: 58; or the HCDR1 comprises an amino acid sequence of SEQ ID NO: 68; the HCDR2 comprises an amino acid sequence of SEQ ID NO: 69; the HCDR3 comprises an amino acid sequence of SEQ ID NO: 70; the LCDR1 comprises an amino acid sequence of SEQ ID NO: 71; the LCDR2 comprises an amino acid sequence of SEQ ID NO: 36; and the LCDR3 comprises an amino acid sequence of SEQ ID NO: 72; or the HCDR1 comprises an amino acid sequence of SEQ ID NO: 81; the HCDR2 comprises an amino acid sequence of SEQ ID NO: 82; the HCDR3 comprises an amino acid sequence of SEQ ID NO: 83; the LCDR1 comprises an amino acid sequence of SEQ ID NO: 84; the LCDR2 comprises an amino acid sequence of SEQ ID NO: 36; and the LCDR3 comprises an amino acid sequence of SEQ ID NO: 72; or the HCDR1 comprises an amino acid sequence of SEQ ID NO: 9; the HCDR2 comprises an amino acid sequence of SEQ ID NO: 10; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 13; and the LCDR3 comprises an amino acid sequence of SEQ ID NO: 14; or the HCDR1 comprises an amino acid sequence of SEQ ID NO: 9; the HCDR2 comprises an amino acid sequence of SEQ ID NO: 90; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 91; and the LCDR3 comprises an amino acid sequence of SEQ ID NO: 14; or the HCDR1 comprises an amino acid sequence of SEQ ID NO: 9; the HCDR2 comprises an amino acid sequence of SEQ ID NO: 10; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 94; and the LCDR3 comprises an amino acid sequence of SEQ ID NO: 14; or the HCDR1 comprises an amino acid sequence of SEQ ID NO: 97; the HCDR2 comprises an amino acid sequence of SEQ ID NO: 98; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 13; and the LCDR3 comprises an amino acid sequence of SEQ ID NO: 14; or the HCDR1 comprises an amino acid sequence of SEQ ID NO: 40; the HCDR2 comprises an amino acid sequence of SEQ ID NO: 41; the HCDR3 comprises an amino acid sequence of SEQ ID NO: 42; the LCDR1 comprises an amino acid sequence of SEQ ID NO: 43; the LCDR2 comprises an amino acid sequence of SEQ ID NO: 21; and the LCDR3 comprises an amino acid sequence of SEQ ID NO: 44; or the HCDR1 comprises an amino acid sequence of SEQ ID NO: 40; the HCDR2 comprises an amino acid sequence of SEQ ID NO: 41; the HCDR3 comprises an amino acid sequence of SEQ ID NO: 42; the LCDR1 comprises an amino acid sequence of SEQ ID NO: 43; the LCDR2 comprises an amino acid sequence of SEQ ID NO: 21; and the LCDR3 comprises an amino acid sequence of SEQ ID NO: 106; or the HCDR1 comprises an amino acid sequence of SEQ ID NO: 17; the HCDR2 comprises an amino acid sequence of SEQ ID NO: 18; the HCDR3 comprises an amino acid sequence of SEQ ID NO: 19; the LCDR1 comprises an amino acid sequence of SEQ ID NO: 20; the LCDR2 comprises an amino acid sequence of SEQ ID NO: 21; and the LCDR3 comprises an amino acid sequence of SEQ ID NO: 22; or the HCDR1 comprises an amino acid sequence of SEQ ID NO: 17; the HCDR2 comprises an amino acid sequence of SEQ ID NO: 108; the HCDR3 comprises an amino acid sequence of SEQ ID NO: 19; the LCDR1 comprises an amino acid sequence of SEQ ID NO: 20; the LCDR2 comprises an amino acid sequence of SEQ ID NO: 21; and the LCDR3 comprises an amino acid sequence of SEQ ID NO: 109.

In some embodiments, the antibody or antigen-binding fragment thereof further comprises one or more of heavy chain HFR1, HFR2, HFR3 and HFR4, and/or one or more of light chain LFR1, LFR2, LFR3 and LFR4.

In some embodiments, the HFR1 comprises an amino acid sequence of QX₂₄QLQESGPGLVKPX₂₅QTLSLTCTVSGYSIX₂₆ (SEQ ID NO: 240), or a homologous sequence of at least 85% sequence identity thereof, the HFR2 comprises an amino acid sequence of SEQ ID NO: 190, or a homologous sequence of at least 85% sequence identity thereof, the HFR3 comprises an amino acid sequence of RX₂₇TISRDTSKNQFSLKLSSVTAX₂₈DTAX₂₉YYCAR (SEQ ID NO: 241), or a homologous sequence of at least 85% sequence identity thereof, the HFR4 comprises an amino acid sequence of SEQ ID NO: 192, or a homologous sequence of at least 85% sequence identity thereof, the LFR1 comprises an amino acid sequence of SEQ ID NO: 193, or a homologous sequence of at least 85% sequence identity thereof, the LFR2 comprises an amino acid sequence of SEQ ID NO: 194, or a homologous sequence of at least 85% sequence identity thereof, the LFR3 comprises an amino acid sequence of GIPARFSGSGSX₃₀TDFTLTISSLEPEDFAVYYC (SEQ ID NO: 242), or a homologous sequence of at least 85% sequence identity thereof, and the LFR4 comprises an amino acid sequence of SEQ ID NO: 196, or a homologous sequence of at least 85% sequence identity thereof, wherein, X₂₄ is V or I, X₂₅ is S or G, X₂₆ is S or T, X₂₇ is V or I, X₂₈ is A or E, X₂₉ is V or T, X₃₀ is R or G.

In some embodiments, the HFR1 comprises an amino acid sequence of SEQ ID NO: 201, or a homologous sequence of at least 85% sequence identity thereof, the HFR2 comprises an amino acid sequence of WX₃₁RQAPGKX₃₂LEWVX₃₃ (SEQ ID NO: 243), or a homologous sequence of at least 85% sequence identity thereof, the HFR3 comprises an amino acid sequence of RFTISRDDAKNSLYLQMNSLRAEDTAX₃₄YYCTR (SEQ ID NO: 244), or a homologous sequence of at least 85% sequence identity thereof, the HFR4 comprises an amino acid sequence of WGQGX₃₅LVTVSS (SEQ ID NO: 245), or a homologous sequence of at least 85% sequence identity thereof, the LFR1 comprises an amino acid sequence of DIQMTQSPSSVSX₃₆SVGDRVTITC (SEQ ID NO: 246), or a homologous sequence of at least 85% sequence identity thereof, the LFR2 comprises an amino acid sequence of SEQ ID NO: 206, or a homologous sequence of at least 85% sequence identity thereof, the LFR3: GVPSRFSGSGSGTDFTX₃₇TISSX₃₈QPEDFATYYC (SEQ ID NO: 247), or a homologous sequence of at least 85% sequence identity thereof, and the LFR4 comprises an amino acid sequence of SEQ ID NO: 196, or a homologous sequence of at least 85% sequence identity thereof, wherein, X₃₁ is V or I, X₃₂ is R or G, X₃₃ is A or S, X₃₄ is M or V, X₃₅ is A or T, X₃₆ is T or A, X₃₇ is L or F, X₃₈ is L or V.

In some embodiments, the HFR1 comprises an amino acid sequence of X₃₉QLVQSGX₄₀EX₄₁KKPGASVKX₄₂SCKASGYTFT (SEQ ID NO: 248), or a homologous sequence of at least 85% sequence identity thereof, the HFR2 comprises an amino acid sequence of SEQ ID NO: 215, or a homologous sequence of at least 85% sequence identity thereof, the HFR3 comprises an amino acid sequence of RX₄₃X₄₄X₄₅X₄₆X₄₇DTSX₄₈STX₄₉YX₅₀X₅₁X₅₂SSLX₅₃X₅₄EDTAVYFCX₅₅S (SEQ ID NO: 249), or a homologous sequence of at least 85% sequence identity thereof, the HFR4 comprises an amino acid sequence of SEQ ID NO: 213, or a homologous sequence of at least 85% sequence identity thereof, the LFR1 comprises an amino acid sequence of DIQMTQSPSSLSX₅₆SVGDRVTITC (SEQ ID NO: 250), or a homologous sequence of at least 85% sequence identity thereof, the LFR2 comprises an amino acid sequence of WYQQKPGKX₅₇PKLLIY (SEQ ID NO: 251), or a homologous sequence of at least 85% sequence identity thereof, the LFR3 comprises an amino acid sequence of GVPSRFSGSGSGTDFTX₅₅TISSX₅₉QPEDFATYYC (SEQ ID NO: 252), or a homologous sequence of at least 85% sequence identity thereof, and the LFR4 comprises an amino acid sequence of SEQ ID NO: 196, or a homologous sequence of at least 85% sequence identity thereof, wherein, X₃₉ is V or I, X₄₀ is A or P, X₄₁ is V or L, X₄₂ is V or I, X₄₃ is V or F, X₄₄ is T or V, X₄₅ is M or F, X₄₆ is T or S, X₄₇ is R or L, X₄₈ is T or V, X₄₉ is V or A, X₅₀ is M or L, X₅₁ is E or Q, X₅₂ is L or I, X₅₃ is R or K, X₅₄ is S or A, X₅₅ is A or V, X₅₆ is T or A, X₅₇ is A or S, X₅₈ is L or F, X₅₉ is L or V.

In some embodiments, the HFR1 comprises an amino acid sequence of SEQ ID NO: 117, 125, 133, 140, 146, 152, 158, 166, 170, 174, 180 or 185, or a homologous sequence of at least 85% sequence identity thereof, the HFR2 comprises an amino acid sequence of SEQ ID NO: 118, 126, 134, 141, 147, 153, 159, 167, 175 or 181, or a homologous sequence of at least 85% sequence identity thereof, the HFR3 comprises an amino acid sequence of SEQ ID NO: 119, 127, 135, 142, 148, 154, 160, 168, 171, 176, 182 or 186, or a homologous sequence of at least 85% sequence identity thereof, the HFR4 comprises an amino acid sequence of SEQ ID NO: 120, 128, 136, 155 or 161, or a homologous sequence of at least 85% sequence identity thereof, the LFR1 comprises an amino acid sequence of SEQ ID NO: 121, 129, 137, 143, 149, 162, 172, 177, 183 or 187, or a homologous sequence of at least 85% sequence identity thereof, the LFR2 comprises an amino acid sequence of SEQ ID NO: 122, 130, 138, 144, 150, 156, 163, 169 or 178, or a homologous sequence of at least 85% sequence identity thereof, the LFR3 comprises an amino acid sequence of SEQ ID NO: 123, 131, 139, 145, 151, 157, 164, 173, 179, 184 or 188, or a homologous sequence of at least 85% sequence identity thereof, and the LFR4 comprises an amino acid sequence of SEQ ID NO: 124, 132 or 165, or a homologous sequence of at least 85% sequence identity thereof.

In some embodiments, the heavy chain variable region of the antibody or an antigen-binding fragment thereof provided herein comprises the sequence selected from the group consisting of SEQ ID NO: 7, 15, 23, 30, 38, 45, 52, 59, 66, 73, 79, 85 or 88, or a homologous sequence thereof having at least 80% sequence identity yet retaining specific binding affinity to human LAG3.

In some embodiments, the light chain variable region of the antibody or an antigen-binding fragment thereof provided herein comprises the sequence selected from the group consisting of SEQ ID NO: 8, 16, 24, 31, 39, 46, 53, 60, 67, 74, 80, 86, or 89, or a homologous sequence thereof having at least 80% sequence identity yet retaining specific binding affinity to human LAG3.

In some embodiments, in the antibody or an antigen-binding fragment thereof provided herein, the heavy chain variable region comprises the sequence of SEQ ID NO: 7 and the light chain variable region comprises the sequence of SEQ ID NO: 8; or the heavy chain variable region comprises the sequence of SEQ ID NO: 15 and the light chain variable region comprises the sequence of SEQ ID NO: 16; or the heavy chain variable region comprises the sequence of SEQ ID NO: 23 and the light chain variable region comprises the sequence of SEQ ID NO: 24; or the heavy chain variable region comprises the sequence of SEQ ID NO: 30 and the light chain variable region comprises the sequence of SEQ ID NO: 31; or the heavy chain variable region comprises the sequence of SEQ ID NO: 38 and the light chain variable region comprises the sequence of SEQ ID NO: 39; or the heavy chain variable region comprises the sequence of SEQ ID NO: 45 and the light chain variable region comprises the sequence of SEQ ID NO: 46; or the heavy chain variable region comprises the sequence of SEQ ID NO: 52 and the light chain variable region comprises the sequence of SEQ ID NO: 53; or the heavy chain variable region comprises the sequence of SEQ ID NO: 59 and the light chain variable region comprises the sequence of SEQ ID NO: 60; or the heavy chain variable region comprises the sequence of SEQ ID NO: 66 and the light chain variable region comprises the sequence of SEQ ID NO: 67; or the heavy chain variable region comprises the sequence of SEQ ID NO: 73 and the light chain variable region comprises the sequence of SEQ ID NO: 74; or the heavy chain variable region comprises the sequence of SEQ ID NO: 79 and the light chain variable region comprises the sequence of SEQ ID NO: 80; or the heavy chain variable region comprises the sequence of SEQ ID NO: 85 and the light chain variable region comprises the sequence of SEQ ID NO: 86; or the heavy chain variable region comprises the sequence of SEQ ID NO: 88 and the light chain variable region comprises the sequence of SEQ ID NO: 89.

In some embodiments, in the antibody or an antigen-binding fragment thereof provided herein, the heavy chain variable region comprises the sequence of SEQ ID NO: 92 and the light chain variable region comprises the sequence of SEQ ID NO: 93; or the heavy chain variable region comprises the sequence of SEQ ID NO: 95 and the light chain variable region comprises the sequence of SEQ ID NO: 96; or the heavy chain variable region comprises the sequence of SEQ ID NO: 99 and the light chain variable region comprises the sequence of SEQ ID NO: 100; or the heavy chain variable region comprises the sequence of SEQ ID NO: 101 and the light chain variable region comprises the sequence of SEQ ID NO: 102; or the heavy chain variable region comprises the sequence of SEQ ID NO: 103 and the light chain variable region comprises the sequence of SEQ ID NO: 104; or the heavy chain variable region comprises the sequence of SEQ ID NO: 105 and the light chain variable region comprises the sequence of SEQ ID NO: 104; or the heavy chain variable region comprises the sequence of SEQ ID NO: 105 and the light chain variable region comprises the sequence of SEQ ID NO: 107; or the heavy chain variable region comprises the sequence of SEQ ID NO: 110 and the light chain variable region comprises the sequence of SEQ ID NO: 111; or the heavy chain variable region comprises the sequence of SEQ ID NO: 110 and the light chain variable region comprises the sequence of SEQ ID NO: 112; or the heavy chain variable region comprises the sequence of SEQ ID NO: 110 and the light chain variable region comprises the sequence of SEQ ID NO: 113; or the heavy chain variable region comprises the sequence of SEQ ID NO: 114 and the light chain variable region comprises the sequence of SEQ ID NO: 111; or the heavy chain variable region comprises the sequence of SEQ ID NO: 114 and the light chain variable region comprises the sequence of SEQ ID NO: 112; or the heavy chain variable region comprises the sequence of SEQ ID NO: 114 and the light chain variable region comprises the sequence of SEQ ID NO: 113; or the heavy chain variable region comprises the sequence of SEQ ID NO: 115 and the light chain variable region comprises the sequence of SEQ ID NO: 111; or the heavy chain variable region comprises the sequence of SEQ ID NO: 115 and the light chain variable region comprises the sequence of SEQ ID NO: 112; or the heavy chain variable region comprises the sequence of SEQ ID NO: 115 and the light chain variable region comprises the sequence of SEQ ID NO: 113; or the heavy chain variable region comprises the sequence of SEQ ID NO: 116 and the light chain variable region comprises the sequence of SEQ ID NO: 111; or the heavy chain variable region comprises the sequence of SEQ ID NO: 116 and the light chain variable region comprises the sequence of SEQ ID NO: 112; or the heavy chain variable region comprises the sequence of SEQ ID NO: 116 and the light chain variable region comprises the sequence of SEQ ID NO: 113.

In some embodiments, the antibody or an antigen-binding fragment thereof provided herein further comprises one or more amino acid residue substitutions or modifications yet retains specific binding affinity to human LAG3. In some embodiments, at least one of the substitutions or modifications is in one or more of the CDR sequences, and/or in one or more of the non-CDR sequences of the heavy chain variable region or light chain variable region. In some embodiments, at least one of the substitutions is a conservative substitution.

In some embodiments, the antibody or an antigen-binding fragment thereof provided herein further comprises an Fc region, optionally an Fc region of human immunoglobulin (Ig), or optionally an Fc region of human IgG. In some embodiments, the Fc region is derived from human IgG1, IgG2, IgG3, IgG4, IgA1, IgA2 or IgM. In some embodiments, the Fc region is derived from human IgG4. In some embodiments, the Fc region derived from human IgG4 comprises an S228P mutation. In some embodiments, the amino acid sequence of the S228P mutated constant region of IgG4 comprises the sequence of SEQ ID NO: 259.

In some embodiments, the antibody or an antigen-binding fragment thereof provided herein is humanized. In some embodiments, the antibody or an antigen-binding fragment thereof provided herein is a monoclonal antibody, a bispecific antibody, a multi-specific antibody, a recombinant antibody, a chimeric antibody, a labeled antibody, a bivalent antibody, an anti-idiotypic antibody or a fusion protein.

In some embodiments, the antibody or an antigen-binding fragment thereof provided herein is a diabody, a Fab, a Fab′, a F(ab′)2, a Fd, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In some embodiments, the antibody or an antigen-binding fragment thereof of has one or more binding properties to human LAG3 selected from the group consisting of: a) specifically binding to human LAG3 D1-D2 recombinant protein at an EC50 of no more than 0.25 nM (preferably no more than 0.1 nM, more preferably no more than 0.05 nM) as measured by ELISA assay; b) having a binding affinity to human LAG3-expressing cells at an EC50 of no more than 5.5 nM (preferably no more than 2.0 nM, more preferably no more than 1.5 nM, and more preferably no more than 0.5 nM) as measured by FACS assay; c) having a binding affinity to human LAG3 at a Kd of no more than 8 nM (preferably no more than 1 nM, more preferably no more than 0.1 nM) as measured by Bio-Layer Interferometry technology (Octet system); d) having a binding affinity to human LAG3 at a Kd of no more than 0.2 nM (preferably no more than 0.1 nM) as measured by Surface Plasmon Resonance technology (Biacore system).

In some embodiments, the antibody or antigen-binding fragment thereof of has one or more properties selected from the group consisting of: a) the ability of blocking LAG3 and MHC II interaction at an IC50 of no more than 8 nM (preferably no more than 5 nM), as measured by FACS assay, using the antibody or antigen-binding fragment thereof, hFc tagged human LAG3 ECD recombinant protein and Raji cells endogenously expressing MHC II; b) the ability of relieving LAG/MHC II mediated suppression at an IC50 of no more than 10 nM (preferably no more than 5 nM), as measured by cell based NFAT reporter assay; c) the ability of blocking LAG3 and LSECtin interaction with a blocking ratio of no less than 90% (preferably no less than 95%) with a concentration of no more than 20 nM, as measured by competitive ELISA assay; d) the ability of blocking LAG3 and FGL1 interaction at an IC50 of no more than 2 nM (preferably no more than 1.5 nM) and/or with a top blocking ratio of no less than 80%, as measured by competitive ELISA assay; e) the ability of blocking LAG/MHC II inhibited T cell activation, as measured by the secretion level of IL-2, wherein the secretion level of IL-2 is more than 200 pg/ml (preferably more than 400 pg/ml), when the concentration of the antibody or antigen-binding fragment is no more than 4 nM.

In some embodiments, the antibody or antigen-binding fragment thereof of shows more than 30% competition for binding to human LAG3 with an antibody comprising a heavy chain variable region comprising the sequence of SEQ ID NO: 253, and a light chain variable region comprising the sequence of SEQ ID NO: 254.

In some embodiments, the antibody or antigen-binding fragment thereof shows more than 30% competition for binding to human LAG3 with an antibody comprising a heavy chain variable region comprising the sequence of SEQ ID NO: 255, and a light chain variable region comprising the sequence of SEQ ID NO: 256.

In some embodiments, the antibody or antigen-binding fragment thereof of any of the preceding claims, wherein the antibody or antigen-binding fragment thereof has different binding epitopes from BMS-986016, i.e., LAG D1 domain extra loop.

The antibody or an antigen-binding fragment thereof of any one of the preceding claims, which is linked to one or more conjugate moieties. In some embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety, or other anticancer drugs.

In another aspect, the present disclosure provides an isolated polynucleotide encoding the antibody or an antigen-binding fragment thereof of the present disclosure.

In another aspect, the present disclosure provides a vector comprising the isolated polynucleotide of the present disclosure.

In another aspect, the present disclosure provides a host cell comprising the vector of the present disclosure.

In another aspect, the present disclosure provides a pharmaceutical composition, comprising: (i) the antibody or an antigen-binding fragment thereof of the present disclosure and (ii) one or more pharmaceutically acceptable carriers.

In another aspect, the present disclosure provides a method of expressing the antibody or an antigen-binding fragment thereof of the present disclosure, comprising culturing the host cell of the present disclosure under the condition at which the vector of the present disclosure is expressed.

In another aspect, the present disclosure provides a method of treating, preventing or alleviating a disease associated with a suppressed immune system in a subject, comprising administering to the subject a therapeutically effective amount of the antibody or an antigen-binding fragment thereof of the present disclosure and/or the pharmaceutical composition of the present disclosure. In some embodiments, the disease associated with a suppressed immune system is cancer or an infectious disease.

In some embodiments, the cancer is a LAG3 related cancer. In some embodiments, the LAG3 related cancer is selected from the group consisting of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

In some embodiments, the LAG3 related cancer is selected from the group consisting of: colorectal cancer, hepatocellular carcinoma, glioma, kidney cancer, breast cancer, multiple myeloma, bladder cancer, neuroblastoma; sarcoma, non-Hodgkin's lymphoma, non-small cell lung cancer, ovarian cancer, pancreatic cancer and rectal cancer. In some embodiments, the cancer is colon cancer or breast cancer.

In some embodiments, the disease associated with a suppressed immune system is a pathogen-associated disease. In some embodiments, the pathogen-associated disease is selected from the group consisting of: chronic viral, bacterial, fungal and parasitic infections. In some embodiments, the chronic viral infections include infections by Epstein Barr virus, Hepatitis A Virus (HAV); Hepatitis B Virus (HBV); Hepatitis C Virus (HCV); herpes viruses (e.g. HSV-1, HSV-2, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia Zambia, trichinella, Dientamoeba Fragilis, Trypanosoma brucei, Trypanosoma cruzi, and Leishmania donovani.

In another aspect, the present disclosure provides a method of detecting the presence or amount of LAG3 in a sample, comprising contacting the sample with the antibody or an antigen-binding fragment thereof of the present disclosure, and determining the presence or the amount of LAG3 in the sample.

In another aspect, the present disclosure provides a method of selecting a patient with a disease, disorder or condition associated with a suppressed immune system, comprising the steps of: a) contacting a sample obtained from the subject with the antibody or an antigen-binding fragment thereof of the present disclosure; b) determining the presence or amount of LAG3 in the sample; and c) correlating the presence or the amount of LAG3 to existence or status of the LAG3 related disease, disorder or condition in the subject.

In another aspect, the present disclosure provides use of the antibody or an antigen-binding fragment thereof of the present disclosure and/or the pharmaceutical composition of the present disclosure in the manufacture of a medicament for treating, preventing or alleviating cancer in a subject.

### BRIEF DESCRIPTION OF THE DRAWINGS

FIGS. 1A, 1B, 1C and 1D show FACS binding curves of anti-LAG3 antibodies 015c, 023c, BMS-986016, BAP-050, TSR-033 (FIG. 1A), 024c, 029c, 035c, 048c, 057c, 047c (FIG. 1B), 027c, 033c, 039c (FIG. 1C) and 021c, 038c (FIG. 1D) against 293F-human LAG3 cells.

FIG. 2 shows FACS binding of anti-LAG3 antibodies against 293F-cyno LAG3 cells.

FIG. 3 shows LAG3/LSECtin interaction blocking activity of anti-LAG3 antibodies 021c, 023c, 029c and BMS-986016 as measured by competitive ELISA assay. The mean blocking ratios obtained with the indicated antibodies are listed at the top of each column.

FIGS. 4A and 4B show LAG3/FGL1 interaction blocking activity of anti-LAG3 antibodies BMS-986016, BAP-050, 047c, 048c, 057c (FIG. 4A), and 015c, 023c, 024c, 027c, 029c, 033c, 035c, 039c (FIG. 4B) as measured by competitive ELISA assay of format I.

FIGS. 5A and 5B show LAG3/FGL1 interaction blocking activity of anti-LAG3 antibodies BMS-986016, 021c, 023c, 029c (FIG. 5A), and 027c, 047c (FIG. 5B) as measured by competitive ELISA assay of format II.

FIG. 6 shows LAG3/FGL1 interaction blocking activity of anti-LAG3 antibodies BMS-986016, BAP-050, 021c, 023c, and 029c as measured by competitive FACS assay of format III.

FIGS. 7A, 7B, 7C and 7D show blocking activity of anti-LAG3 antibodies BMS-986016, BAP-050, 015c, 023c (FIG. 7A), 024c, 027c, 033c, 035c (FIG. 7B), 039c, 047c, 048c, 057c (FIG. 7C), and TSR-033, 021c, 029c, 038c (FIG. 7D) as measured by NFAT reporter assay.

FIGS. 8A, 8B, 8C, 8D, 8E and 8F show functional activity of anti-LAG3 antibodies BMS-986016, BAP-050, 015c (FIG. 8A), 024c, 027c, 033c, 035c (FIG. 8B), 023c, 038c (FIG. 8C), 021c, 029c, 057c (FIG. 8D), 039c, 047c, 048c (FIG. 8E), and TSR-033 (FIG. 8F) as measured by antigen specific T cell activation assay.

FIGS. 9A and 9B show binding curves of anti-LAG3 antibodies to LAG3 D1 domain extra loop peptide as measured by ELISA assay of format I (FIG. 9A) or format II (FIG. 9B).

FIGS. 10A and 10B show the results of an mouse breast carcinoma syngeneic model to assess in vivo activity of anti-LAG3 treatment. FIG. 10A shows the average tumor growth curves for each study group, and FIG. 10B shows the individual tumor growth curves for each mouse. **p<0.01, ****p<0.0001.

FIG. 11 shows FACS binding curves of 023c originated humanized antibodies against 293F-human LAG3 cells.

FIG. 12 shows LAG3/MHC II interaction blocking activity of 023c originated humanized antibodies as measured by competitive FACS assay.

FIG. 13 shows LAG3/FGL1 interaction blocking activity of 023c originated humanized antibodies as measured by competitive ELISA assay.

FIG. 14 shows blocking activity of hu023.04 and hu023.11 as measured by NFAT reporter assay.

FIG. 15 shows functional activity of hu023.04 and hu023.11 as measured by antigen specific T cell activation assay.

FIG. 16 shows FACS binding curves of 021c originated humanized antibodies against CHOK1-human LAG3 cells.

FIG. 17 shows LAG3/MHC II interaction blocking activity of 021c originated humanized antibodies as measured by competitive FACS assay. The mean blocking ratios obtained with the indicated antibodies are listed at the top of each column.

FIG. 18 shows blocking activity of 021c originated humanized antibodies as measured by NFAT reporter assay.

FIG. 19 shows functional activity of 021c originated humanized antibodies as measured by antigen specific T cell activation assay.

FIGS. 20A and 20B show FACS binding curves of hu029.40, hu029.53, hu029.55 (FIG. 20A) and hu029.55.S93Q (FIG. 20B) against CHOK1-human LAG3 cells.

FIGS. 21A and 21B show LAG3/MHC II interaction blocking activity of hu029.40, hu029.53, hu029.55 (FIG. 21A) and hu029.55.S93Q (FIG. 21B) as measured by competitive FACS assay. The mean blocking ratios obtained with the indicated antibodies are listed at the top of each column.

FIGS. 22A and 22B show blocking activity of hu029.40, hu029.53, hu029.55 (FIG. 22A) and hu029.55.S93Q (FIG. 22B) as measured by NFAT reporter assay.

FIGS. 23A and 23B show functional activity of hu029.40, hu029.53, hu029.55 (FIG. 23A) and hu029.55.S93Q (FIG. 23B) as measured by antigen specific T cell activation assay.

FIGS. 24A, 24B, 24C, 24D, 24E and 24F show the results of an mouse breast carcinoma syngeneic model to assess in vivo activity of anti-LAG3 humanized antibodies treatment. FIG. 24A to FIG. 24E show the average tumor growth curves for each study group, and FIG. 24F shows the individual tumor growth curves for each mouse in the indicated groups. *p<0.05, **p<0.01, ***p<0.001, ****p<0.0001, ns stands for not significant (p>0.05).

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879 (1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}-V_{H}) (see, e.g. Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementarity domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another V_{H}-V_{L} moiety such that V_{H}'S of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g. a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The term “affinity” as used herein refers to the strength of non-covalent interaction between an immunoglobulin molecule (i.e., antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to, surface plasmon resonance method, microscale thermophoresis method, HPLC-MS method and flow cytometry (such as FACS) method. A K_{D} value of ≤10⁻⁶ M (e.g. ≤5×10⁻⁷ M, ≤2×10⁻⁷ M, ≤10⁻⁷ M, ≤5×10⁻⁸ M, ≤2×10⁻⁸ M, ≤10⁻⁸M, ≤5×10⁻⁹ M, ≤4×10⁻⁹M, ≤3×10⁻⁹M, ≤2×10⁻⁹ M, or ≤10⁻⁹ M) can indicate specific binding between an antibody or antigen binding fragments thereof and LAG3 (e.g. human LAG3).

The ability to “compete for binding to LAG3” as used herein refers to the ability of a first antibody or antigen-binding fragment to inhibit the binding interaction between human LAG3 and a second antibody to any detectable degree. In certain embodiments, an antibody or antigen-binding fragment that compete for binding to human LAG3 inhibits the binding interaction between human LAG3 and a second anti-LAG3 antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Name of Amino Acid | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementarity strand) or amino acid sequences that have sequence identity of at least 60% (e.g. at least 65%, 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or an antigen-binding fragment thereof, at least one promoter (e.g. SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a LAG3 related disease, or refer to identification of a subject with a LAG3 related disease who may benefit from a particular treatment regimen. In some embodiments, diagnosis contains the identification of abnormal amount or activity of LAG3. In some embodiments, diagnosis refers to the identification of a cancer or an autoimmune disease in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject.

As used herein, “LAG3” or “LAG-3” are used interchangeably, referring to Lymphocyte-activation gene 3, a protein in humans encoded by the LAG3 gene (Gene ID: 3902, in NCBI Gene Database). LAG3 was discovered in 1990 (Triebel F et al. (May 1990), LAG-3, a novel lymphocyte activation gene closely related to CD4, The Journal of Experimental Medicine. 171 (5): 1393-405) and was designated CD223 (cluster of differentiation 223) after the Seventh Human Leucocyte Differentiation Antigen Workshop in 2000 (Mason D et al. (November 2001), CD antigens 2001, Journal of Leukocyte Biology. 70 (5): 685-90), is a cell surface molecule with diverse biologic effects on T cell function. It is an immune checkpoint receptor and as such is the target of various drug development programs by pharmaceutical companies seeking to develop new treatments for cancer and autoimmune disorders. In soluble form it is also being developed as a cancer drug in its own right (Syn, Nicholas L et al. (December 2017), De-novo and acquired resistance to immune checkpoint targeting, The Lancet Oncology. 18 (12): e731-e741).

The term “anti-LAG3 antibody” refers to an antibody that is capable of specific binding to LAG3 (e.g., human or mouse LAG3). The term “anti-human LAG3 antibody” refers to an antibody that is capable of specific binding to human LAG3.

A “disease associated with a suppressed immune system” as used herein refers to a LAG3 related disease, especially cancer or an infection.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

### Anti-LAG3 Antibodies

The present disclosure provides anti-LAG3 antibodies and antigen-binding fragments thereof. The anti-LAG3 antibodies and antigen-binding fragments provided herein are capable of specific binding to LAG3.

Binding of the antibodies or the antigen-binding fragments thereof provided herein to human LAG3 can be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), flow cytometry assay, and other binding assay. In certain embodiments, the antibodies and the antigen-binding fragments thereof provided herein specifically bind to human LAG3 at an EC₅₀ (i.e., 50% binding concentration) of no more than 1 nM, no more than 0.9 nM, no more than 0. 8 nM, no more than 0.7 nM, no more than 0.6 nM, no more than 0.5 nM, no more than 0.4 nM, no more than 0.3 nM, no more than 0.2 nM, no more than 0.1 nM, no more than 0.09 nM, no more than 0.08 nM, no more than 0.07 nM, no more than 0.06 nM or no more than 0.05 nM by ELISA. In certain embodiments, the EC₅₀ value is measured by the method as described in Example 3.2 of the present disclosure.

In certain embodiments, the antibodies and the antigen-binding fragments thereof provided herein specifically bind to human LAG3 D1-D2 recombinant protein at an EC₅₀ of no more than 1 nM (e.g. no more than 5×10⁻¹⁰ M, no more than 3×10⁻¹⁰ M, no more than 1×10⁻¹⁰ M) as measured by ELISA assay. In certain embodiments, the antibodies and the antigen-binding fragments thereof provided herein specifically bind to human LAG3 D1-D2 recombinant protein at an EC₅₀ of no more than 1 nM (e.g., no more than 5×10⁻¹⁰ M, no more than 3×10⁻¹⁰ M, no more than 1×10⁻¹⁰ M) as measured by ELISA assay.

In certain embodiments, the antibodies and antigen-binding fragments thereof provided herein bind to LAG3 D1-D2 recombinant protein at an EC₅₀ of no more than 50 nM (e.g. no more than 40 nM, no more than 30 nM, no more than 20 nM, no more than 10 nM, no more than 1 nM) as measured by ELISA assay.

In certain embodiments, the antibodies and antigen-binding fragments thereof provided herein bind to human LAG3-expressing cells at an EC₅₀ of no more than 5.5 nM (preferably no more than 2.0 nM, more preferably no more than 1.5 nM, and more preferably no more than 0.5 nM) as measured by FACS assay. In certain embodiments, the EC₅₀ value is measured by the method as described in Example 3.3 of the present disclosure.

In certain embodiments, the antibodies and the antigen-binding fragments thereof provided herein specifically bind to human LAG3 at an K_{D} value no more than 8 nM (preferably no more than 1 nM, more preferably no more than 0.1 nM) as measured by Bio-Layer Interferometry (BLI) technology. BLI technology is based on a label-free technology for measuring biomolecular interactions, see, for example, those described in Cooper, Matthew (May 7, 2006), Current biosensor technologies in drug discovery, Drug Discovery World (Summer): 68-82, or Rich, Rebecca L et al. (1 Feb. 2007), Higher-throughput, label-free, real-time molecular interaction analysis, Analytical Biochemistry. 361(1): 1-6. In certain embodiments, the K_{D}value is measured by the method as described in Example 4.2.8 of the present disclosure.

In certain embodiments, the antibodies and the antigen-binding fragments thereof provided herein specifically bind to human LAG3 at an K_{D} value no more than 0.2 nM (preferably no more than 0.1 nM) assay. Biacore assay is based on surface plasmon resonance technology, see, for example, Murphy, M. et al., Current protocols in protein science, Chapter 19, unit 19.14, 2006. In certain embodiments, the K_{D} value is measured by the method as described in Example 5.1.2.6 of the present disclosure.

In certain embodiments, the antibodies and the antigen-binding fragments thereof provided herein have the ability of blocking LAG3 and MHC II interaction at an IC₅₀ of no more than 8 nM (preferably no more than 5 nM), as measured by FACS assay, using the antibody or antigen-binding fragment thereof, hFc tagged human LAG3 ECD recombinant protein and Raji cells endogenously expressing MHC II. In certain embodiments, the IC₅₀ value is measured by the method as described in Example 3.5 of the present disclosure.

In certain embodiments, the antibodies and the antigen-binding fragments thereof provided herein have the ability of relieving LAG/MIIC II mediated suppression at an IC₅₀ of no more than 10 nM (preferably no more than 5 nM), as measured by cell based NFAT reporter assay. In certain embodiments, the IC₅₀ value is measured by the method as described in Example 3.6 of the present disclosure.

In certain embodiments, the antibodies and the antigen-binding fragments thereof provided herein have the ability of blocking LAG3 and LSECtin interaction with a blocking ratio of no less than 90% (preferably no less than 95%) with a concentration of no more than 20 nM, as measured by competitive ELISA assay. In certain embodiments, the blocking ratio is measured by the method as described in Example 4.2.3 of the present disclosure.

In certain embodiments, the antibodies and the antigen-binding fragments thereof provided herein have the ability of blocking LAG3 and LSECtin interaction at an IC50 of no more than 2 nM (preferably no more than 1.5 nM) and/or with a top blocking ratio of no less than 80%, as measured by competitive ELISA assay. In certain embodiments, the blocking ratio is measured by the method as described in Example 4.2.4 of the present disclosure.

In certain embodiments, the antibodies and the antigen-binding fragments thereof provided herein have the ability of blocking LAG/MHC II inhibited T cell activation, as measured by the secretion level of IL-2, wherein the secretion level of IL-2 is more than 200 μg/ml (preferably more than 400 μg/ml), when the concentration of the antibody or antigen-binding fragment is no more than 4 nM. In certain embodiments, the blocking ability is measured by the method as described in Example 3.7 of the present disclosure.

### Illustrative Anti-LAG3 Antibodies

In certain embodiments, the present disclosure provides anti-LAG3 antibodies and antigen-binding fragments thereof comprising one or more (e.g. 1, 2, 3, 4, 5, or 6) CDRs comprising the sequences selected from the group consisting of SYGX₁N (SEQ ID NO: 226), EIYPRSGNTYYNEX₂X₃X₄X₅(SEQ ID NO: 227), GGTYDGYYYAMDX₆ (SEQ ID NO: 228), RASESVDNFGSSFX₇H (SEQ ID NO: 229), SEQ ID NO: 13, SEQ ID NO: 58, DYNX₈N (SEQ ID NO: 230), LVDPIYGTIRYNQX₉FKX₁₀ (SEQ ID NO: 231), IX₁₁TX₁₂VRYFDX₁₃ (SEQ ID NO: 232), RSSX₁₄NIVHX₁₅DGNTYLE (SEQ ID NO: 233), SEQ ID NO: 36, SEQ ID NO: 72, SGYYWX₁₆ (SEQ ID NO: 234), DISYX₁₇X₁₈GNNYNPSLKN (SEQ ID NO: 235), SEQ ID NO: 11, SEQ ID NO: 12, RASNX₁₉EX₂₀ (SEQ ID NO: 236), SEQ ID NO: 14, SEQ ID NO: 40, SEQ ID NO: 41, SEQ ID NO: 42, SEQ ID NO: 43, SEQ ID NO: 21, QQHDX₂₁SPWT (SEQ ID NO: 237), SEQ ID NO: 17, X₂₂IYTDTGEPTYAEEFKG (SEQ ID NO: 238), SEQ ID NO: 19, SEQ ID NO: 20, SEQ ID NO: 21, QQHYNX₂₃PPT (SEQ ID NO: 239), SEQ ID NO: 1, SEQ ID NO: 2, SEQ ID NO: 3, SEQ ID NO: 4, SEQ ID NO: 5, SEQ ID NO: 6, SEQ ID NO: 1, SEQ ID NO: 25, SEQ ID NO: 26, SEQ ID NO: 27, SEQ ID NO: 28, SEQ ID NO: 29, SEQ ID NO: 32, SEQ ID NO: 33, SEQ ID NO: 34, SEQ ID NO: 35, SEQ ID NO: 36, SEQ ID NO: 37, SEQ ID NO: 47, SEQ ID NO: 48, SEQ ID NO: 49, SEQ ID NO: 50, SEQ ID NO: 36, SEQ ID NO: 51, SEQ ID NO: 9, SEQ ID NO: 61, SEQ ID NO: 62, SEQ ID NO: 63, SEQ ID NO: 64, SEQ ID NO: 65, wherein X₁ is I or V, X₂ is K or R, X₃ is F or L, X₄ is K or G, X₅ is D or G, X₆ is F or Y, X₇ is L or M, X₈ is L or M, X₉ is N or K, X₁₀ is D or G, X₁₁ is M or T, X₁₂ is A or S, X₁₃ is H or Y, X₁₄ is L or Q, X₁₅ is S or T, X₁₆ is N or T, X₁₇ is E or D, X₁₈ is G or A, X₁₉ is L or R, X₂₀ is T or S, X₂₁ is S or Q, X₂₂ is M or I, X₂₃ is A or S.

“Antibody 015” as used herein refers to a monoclonal antibody comprising a heavy chain variable region having the sequence of SEQ ID NO: 7, and a light chain variable region having the sequence of SEQ ID NO: 8.

“Antibody 021” as used herein refers to a monoclonal antibody comprising a heavy chain variable region having the sequence of SEQ ID NO: 15, and a light chain variable region having the sequence of SEQ ID NO: 16.

“Antibody 023” as used herein refers to a monoclonal antibody comprising a heavy chain variable region having the sequence of SEQ ID NO: 23, and a light chain variable region having the sequence of SEQ ID NO: 24.

“Antibody 024” as used herein refers to a monoclonal antibody comprising a heavy chain variable region having the sequence of SEQ ID NO: 30, and a light chain variable region having the sequence of SEQ ID NO: 31.

“Antibody 027” as used herein refers to a monoclonal antibody comprising a heavy chain variable region having the sequence of SEQ ID NO: 38, and a light chain variable region having the sequence of SEQ ID NO: 39.

“Antibody 029” as used herein refers to a monoclonal antibody comprising a heavy chain variable region having the sequence of SEQ ID NO: 45, and a light chain variable region having the sequence of SEQ ID NO: 46.

“Antibody 033” as used herein refers to a monoclonal antibody comprising a heavy chain variable region having the sequence of SEQ ID NO: 52, and a light chain variable region having the sequence of SEQ ID NO: 53.

“Antibody 035” as used herein refers to a monoclonal antibody comprising a heavy chain variable region having the sequence of SEQ ID NO: 59, and a light chain variable region having the sequence of SEQ ID NO: 60.

“Antibody 038” as used herein refers to a monoclonal antibody comprising a heavy chain variable region having the sequence of SEQ ID NO: 66, and a light chain variable region having the sequence of SEQ ID NO: 67.

“Antibody 039” as used herein refers to a monoclonal antibody comprising a heavy chain variable region having the sequence of SEQ ID NO: 73, and a light chain variable region having the sequence of SEQ ID NO: 74.

“Antibody 047” as used herein refers to a monoclonal antibody comprising a heavy chain variable region having the sequence of SEQ ID NO: 79, and a light chain variable region having the sequence of SEQ ID NO: 80.

“Antibody 048” as used herein refers to a monoclonal antibody comprising a heavy chain variable region having the sequence of SEQ ID NO: 85, and a light chain variable region having the sequence of SEQ ID NO: 86.

“Antibody 057” as used herein refers to a monoclonal antibody comprising a heavy chain variable region having the sequence of SEQ ID NO: 88, and a light chain variable region having the sequence of SEQ ID NO: 89.

In certain embodiments, the present disclosure provides anti-LAG3 antibodies and antigen-binding fragments thereof comprising one or more (e.g. 1, 2, 3, 4, 5, or 6) CDRs sequences of Antibody 015, 021, 023, 024, 027, 029, 033, 035, 038, 039, 047, 048 or 057.

In certain embodiments, the present disclosure provides anti-LAG3 antibodies and antigen-binding fragments thereof comprising HCDR1 comprising the sequence selected from the group consisting of SEQ ID NOs: 1, 9, 17, 1, 32, 40, 47, 54, 9, 68, 75, 81 and 54, HCDR2 comprising the sequence selected from the group consisting of SEQ ID NOs: 2, 10, 18, 25, 33, 41, 48, 55, 61, 69, 76, 82 and 87, and HCDR3 comprising the sequence selected from the group consisting of SEQ ID NOs: 3, 11, 19, 26, 34, 42, 49, 56, 62, 70, 77, 83 and 77, and/or LCDR1 comprising the sequence selected from the group consisting of SEQ ID NOs: 4, 12, 20, 27, 35, 43, 50, 57, 63, 71, 78, 84 and 78, LCDR2 comprising the sequence selected from the group consisting of SEQ ID NOs: 5, 13, 21, 28, 36, 21, 36, 13, 64, 36, 13, 36 and 13, and LCDR3 comprising the sequence selected from the group consisting of SEQ ID NOs: 6, 14, 22, 29, 37, 44, 51, 58, 65, 72, 58, 72 and 58.

In certain embodiments, the present disclosure provides anti-LAG3 antibodies and antigen-binding fragments thereof comprising a HCDR1 comprising the sequence of SEQ ID NO: 1, a HCDR2 comprising the sequence of SEQ ID NO: 2, a HCDR3 comprising the sequence of SEQ ID NO: 3, and/or a LCDR1 comprising the sequence of SEQ ID NO: 4, a LCDR2 comprising the sequence of SEQ ID NO: 5, and a LCDR3 comprising the sequence of SEQ ID NO: 6.

In certain embodiments, the present disclosure provides anti-LAG3 antibodies and antigen-binding fragments thereof comprising a HCDR1 comprising the sequence of SEQ ID NO: 9, a HCDR2 comprising the sequence of SEQ ID NO: 10, a HCDR3 comprising the sequence of SEQ ID NO: 11, and/or a LCDR1 comprising the sequence of SEQ ID NO: 12, a LCDR2 comprising the sequence of SEQ ID NO: 13, and a LCDR3 comprising the sequence of SEQ ID NO: 14.

In certain embodiments, the present disclosure provides anti-LAG3 antibodies and antigen-binding fragments thereof comprising a HCDR1 comprising the sequence of SEQ ID NO: 17, a HCDR2 comprising the sequence of SEQ ID NO: 18, a HCDR3 comprising the sequence of SEQ ID NO: 19, and/or a LCDR1 comprising the sequence of SEQ ID NO: 20, a LCDR2 comprising the sequence of SEQ ID NO: 21, and a LCDR3 comprising the sequence of SEQ ID NO: 22.

In certain embodiments, the present disclosure provides anti-LAG3 antibodies and antigen-binding fragments thereof comprising a HCDR1 comprising the sequence of SEQ ID NO: 1, a HCDR2 comprising the sequence of SEQ ID NO: 25, a HCDR3 comprising the sequence of SEQ ID NO: 26, and/or a LCDR1 comprising the sequence of SEQ ID NO: 27, a LCDR2 comprising the sequence of SEQ ID NO: 28, and a LCDR3 comprising the sequence of SEQ ID NO: 29.

In certain embodiments, the present disclosure provides anti-LAG3 antibodies and antigen-binding fragments thereof comprising a HCDR1 comprising the sequence of SEQ ID NO: 32, a HCDR2 comprising the sequence of SEQ ID NO: 33, a HCDR3 comprising the sequence of SEQ ID NO: 34, and/or a LCDR1 comprising the sequence of SEQ ID NO: 35, a LCDR2 comprising the sequence of SEQ ID NO: 36, and a LCDR3 comprising the sequence of SEQ ID NO: 37.

In certain embodiments, the present disclosure provides anti-LAG3 antibodies and antigen-binding fragments thereof comprising a HCDR1 comprising the sequence of SEQ ID NO: 40, a HCDR2 comprising the sequence of SEQ ID NO: 41, a HCDR3 comprising the sequence of SEQ ID NO: 42, and/or a LCDR1 comprising the sequence of SEQ ID NO: 43, a LCDR2 comprising the sequence of SEQ ID NO: 21, and a LCDR3 comprising the sequence of SEQ ID NO: 44.

In certain embodiments, the present disclosure provides anti-LAG3 antibodies and antigen-binding fragments thereof comprising a HCDR1 comprising the sequence of SEQ ID NO: 47, a HCDR2 comprising the sequence of SEQ ID NO: 48, a HCDR3 comprising the sequence of SEQ ID NO: 49, and/or a LCDR1 comprising the sequence of SEQ ID NO: 50, a LCDR2 comprising the sequence of SEQ ID NO: 36, and a LCDR3 comprising the sequence of SEQ ID NO: 51.

In certain embodiments, the present disclosure provides anti-LAG3 antibodies and antigen-binding fragments thereof comprising a HCDR1 comprising the sequence of SEQ ID NO: 54, a HCDR2 comprising the sequence of SEQ ID NO: 55, a HCDR3 comprising the sequence of SEQ ID NO: 56, and/or a LCDR1 comprising the sequence of SEQ ID NO: 57, a LCDR2 comprising the sequence of SEQ ID NO: 13, and a LCDR3 comprising the sequence of SEQ ID NO: 58.

In certain embodiments, the present disclosure provides anti-LAG3 antibodies and antigen-binding fragments thereof comprising a HCDR1 comprising the sequence of SEQ ID NO: 9, a HCDR2 comprising the sequence of SEQ ID NO: 61, a HCDR3 comprising the sequence of SEQ ID NO: 62, and/or a LCDR1 comprising the sequence of SEQ ID NO: 63, a LCDR2 comprising the sequence of SEQ ID NO: 64, and a LCDR3 comprising the sequence of SEQ ID NO: 65.

In certain embodiments, the present disclosure provides anti-LAG3 antibodies and antigen-binding fragments thereof comprising a HCDR1 comprising the sequence of SEQ ID NO: 68, a HCDR2 comprising the sequence of SEQ ID NO: 69, a HCDR3 comprising the sequence of SEQ ID NO: 70, and/or a LCDR1 comprising the sequence of SEQ ID NO: 71, a LCDR2 comprising the sequence of SEQ ID NO: 36, and a LCDR3 comprising the sequence of SEQ ID NO: 72.

In certain embodiments, the present disclosure provides anti-LAG3 antibodies and antigen-binding fragments thereof comprising a HCDR1 comprising the sequence of SEQ ID NO: 75, a HCDR2 comprising the sequence of SEQ ID NO: 76, a HCDR3 comprising the sequence of SEQ ID NO: 77, and/or a LCDR1 comprising the sequence of SEQ ID NO: 78, a LCDR2 comprising the sequence of SEQ ID NO: 13, and a LCDR3 comprising the sequence of SEQ ID NO: 58.

In certain embodiments, the present disclosure provides anti-LAG3 antibodies and antigen-binding fragments thereof comprising a HCDR1 comprising the sequence of SEQ ID NO: 81, a HCDR2 comprising the sequence of SEQ ID NO: 82, a HCDR3 comprising the sequence of SEQ ID NO: 83, and/or a LCDR1 comprising the sequence of SEQ ID NO: 84, a LCDR2 comprising the sequence of SEQ ID NO: 36, and a LCDR3 comprising the sequence of SEQ ID NO: 72.

In certain embodiments, the present disclosure provides anti-LAG3 antibodies and antigen-binding fragments thereof comprising a HCDR1 comprising the sequence of SEQ ID NO: 54, a HCDR2 comprising the sequence of SEQ ID NO: 87, a HCDR3 comprising the sequence of SEQ ID NO: 77, and/or a LCDR1 comprising the sequence of SEQ ID NO: 78, a LCDR2 comprising the sequence of SEQ ID NO: 13, and a LCDR3 comprising the sequence of SEQ ID NO: 58.

Table 1 below shows the CDR amino acid sequences of antibodies 015, 021, 023, 024, 027, 029, 033, 035, 038, 039, 047, 048 and 057. The CDR boundaries were defined or identified by the convention of Kabat. Table 2 below shows the heavy chain and light chain variable region amino acid sequences of antibodies 015, 021, 023, 024, 027, 029, 033, 035, 038, 039, 047, 048 and 057. Table 3 below shows the framework region amino acid sequences of antibodies 015, 021, 023, 024, 027, 029, 033, 035, 038, 039, 047, 048 and 057.

TABLE 1
CDR amino acid sequences of 13 antibodies

| Antibody | | CDR1 | CDR2 | CDR3 |
|---|---|---|---|---|
| 015 | HCDR | SEQ ID NO: 1 | SEQ ID NO: 2 | SEQ ID NO: 3 |
| | | DYYMN | VINPFNGDTAY | DDGYYLYYFDS |
| | | | KQKFKG | |
| | LCDR | SEQ ID NO: 4 | SEQ ID NO: 5 | SEQ ID NO: 6 |
| | | RASQDIGSRLN | ATSSLDS | LQYASSPPT |
| 021 | HCDR | SEQ ID NO: 9 | SEQ ID NO: 10 | SEQ ID NO: 11 |
| | | SGYYWN | DISYDGGNNY | DITLTGAMDY |
| | | | NPSLKN | |
| | LCDR | SEQ ID NO: 12 | SEQ ID NO: 13 | SEQ ID NO: 14 |
| | | RASESVDNYGI | RASNLES | QQSNKDPYT |
| | | SFMH | | |
| 023 | HCDR | SEQ ID NO: 17 | SEQ ID NO: 18 | SEQ ID NO: 19 |
| | | ESPMN | MIYTDTGEPTY | DYGSILYWYFD |
| | | | AEEFKG | V |
| | LCDR | SEQ ID NO: 20 | SEQ ID NO: 21 | SEQ ID NO: 22 |
| | | KASQDVSTAV | SASYRYT | QQHYNSPPT |
| | | A | | |
| 024 | HCDR | SEQ ID NO: 1 | SEQ ID NO: 25 | SEQ ID NO: 26 |
| | | DYYMN | DIDPNNGGTSY | AGSYYYGSSYV |
| | | | NQKFKG | EYFDV |
| | LCDR | SEQ ID NO: 27 | SEQ ID NO: 28 | SEQ ID NO: 29 |
| | | RASGNIHNYLA | NAKTLAD | QHFWNTPYT |
| 027 | HCDR | SEQ ID NO: 32 | SEQ ID NO: 33 | SEQ ID NO: 34 |
| | | RYWMS | EINPDSSTINYA | LGIIPTYHYGSSG |
| | | | PSLKD | YYYAMDY |
| | LCDR | SEQ ID NO: 35 | SEQ ID NO: 36 | SEQ ID NO: 37 |
| | | RSSQTIVHGNG | KVSNRFS | FQVSHVPYT |
| | | HTSLE | | |
| 029 | HCDR | SEQ ID NO: 40 | SEQ ID NO: 41 | SEQ ID NO: 42 |
| | | SYAMS | YVTSGGDYIYY | VAYDYEWYFDV |
| | | | ADTVKG | |
| | LCDR | SEQ ID NO: 43 | SEQ ID NO: 21 | SEQ ID NO: 44 |
| | | KASQDVTTAV | SASYRYT | QQHDSSPWT |
| | | A | | |
| 033 | HCDR | SEQ ID NO: 47 | SEQ ID NO: 48 | SEQ ID NO: 49 |
| | | DDYIH | WIDLENGDTEY | KGSH |
| | | | ASKFQG | |
| | LCDR | SEQ ID NO: 50 | SEQ ID NO: 36 | SEQ ID NO: 51 |
| | | RSSQSLVYSNG | KVSNRFS | SQSTHVPYT |
| | | NTYLN | | |
| 035 | HCDR | SEQ ID NO: 54 | SEQ ID NO: 55 | SEQ ID NO: 56 |
| | | SYGIN | EIYPRSGNTYY | GGTYDGYYYA |
| | | | NEKFKD | MDF |
| | LCDR | SEQ ID NO: 57 | SEQ ID NO: 13 | SEQ ID NO: 58 |
| | | RASESVDNFGS | RASNLES | QQVNKDPRT |
| | | SFLH | | |
| 038 | HCDR | SEQ ID NO: 9 | SEQ ID NO: 61 | SEQ ID NO: 62 |
| | | SGYYWN | YISYDGSNEYN | EEDGYYGGMDY |
| | | | LSLKN | |
| | LCDR | SEQ ID NO: 63 | SEQ ID NO: 64 | SEQ ID NO: 65 |
| | | RASQDVDTAV | WASTRHT | QQYSSYPYT |
| | | A | | |
| 039 | HCDR | SEQ ID NO: 68 | SEQ ID NO: 69 | SEQ ID NO: 70 |
| | | DYNLN | LVDPIYGTIRY | IMTAVRYFDH |
| | | | NONFKD | |
| | LCDR | SEQ ID NO: 71 | SEQ ID NO: 36 | SEQ ID NO: 72 |
| | | RSSLNIVHSDG | KVSNRFS | FQGSHVPPT |
| | | NTYLE | | |
| 047 | HCDR | SEQ ID NO: 75 | SEQ ID NO: 76 | SEQ ID NO: 77 |
| | | SYGVN | EIYPRSGNTYY | GGTYDGYYYA |
| | | | NERFGD | MDY |
| | LCDR | SEQ ID NO: 78 | SEQ ID NO: 13 | SEQ ID NO: 58 |
| | | RASESVDNFGS | RASNLES | QQVNKDPRT |
| | | SFMH | | |
| 048 | HCDR | SEQ ID NO: 81 | SEQ ID NO: 82 | SEQ ID NO: 83 |
| | | DYNMN | LVDPIYGTIRY | ITTSVRYFDY |
| | | | NQKFKG | |
| | LCDR | SEQ ID NO: 84 | SEQ ID NO: 36 | SEQ ID NO: 72 |
| | | RSSQNIVHTDG | KVSNRFS | FQGSHVPPT |
| | | NTYLE | | |
| 057 | HCDR | SEQ ID NO: 54 | SEQ ID NO: 87 | SEQ ID NO: 77 |
| | | SYGIN | EIYPRSGNTYY | GGTYDGYYYA |
| | | | NEKLKG | MDY |
| | LCDR | SEQ ID NO: 78 | SEQ ID NO: 13 | SEQ ID NO: 58 |
| | | RASESVDNFGS | RASNLES | QQVNKDPRT |
| | | SFMH | | |

TABLE 2
Variable region amino acid sequences of 13 antibodies

| Antibody | VH | VL |
|---|---|---|
| 015 | SEQ ID NO: 7 | SEQ ID NO: 8 |
| | EVQLQQSGPVLVKPGASVKLSC | DIQMTQSPSSLSASLGERVS |
| | KASGFTFTDYYMNWVKLSHGK | LTCRASQDIGSRLNWLQQE |
| | SLEWIGVINPFNGDTAYKQKFK | PDGTIKRLIYATSSLDSGVP |
| | GKATLTVDKSSSTAYMELNSLT | KRFSGSRSGSDYSLTISSLD |
| | SEDSAVYYCARDDGYYLYYFDS | SEDFVVYYCLQYASSPPTF |
| | WGQGTTLTVSS | GAGTKLELK |
| 021 | SEQ ID NO: 15 | SEQ ID NO: 16 |
| | DIQLQESGPGLVKPSQSLSLTCS | DIVLTQSPASLAVSLGQRA |
| | VTGYSITSGYYWNWIRQFPGNK | TISCRASESVDNYGISFMH |
| | LEWMGDISYDGGNNYNPSLKNR | WYQQRPGQPPKLLIYRASN |
| | ISITRDTSKNQFFLKLNSVTTEDTATY | LESGIPARFSGSGSRTDFTL |
| | YCARDITLTGAMDYWGQG | TINPVETDDVATYYCQQSN |
| | TSVTVSS | KDPYTFGGGTKLEIK |
| 023 | SEQ ID NO: 23 | SEQ ID NO: 24 |
| | QIQLVQSGPVLKKPGETVKISCK | DIVMTQSHKFMSTSVGDR |
| | ASGYTFTESPMNWVKQAPGRGF | VSITCKASQDVSTAVAWY |
| | KWMGMIYTDTGEPTYAEEFKGR | QQTPGQSPKLLIYSASYRY |
| | FAFSLETSASTAYLQINNLKSED | TGVPDRFTGSGSGTDFTFTI |
| | TATYFCVSDYGSILYWYFDVWG | SSVQAEDLAVYYCQQHYN |
| | TGTTVTVSS | SPPTFGAGTKLELK |
| 024 | SEQ ID NO: 30 | SEQ ID NO: 31 |
| | EVQLQQSGPELVKPGASVKISCK | DIQMTQSPASLSASVGETV |
| | ASGYTFTDYYMNWVKQSHGKR | TITCRASGNIHNYLAWYQQ |
| | LEWIGDIDPNNGGTSYNQKFKG | KQGASPQLLVYNAKTLAD |
| | KATLTVDKSSSSAYMDLRSLTSE | GVPSRFSGSGSGTEFSLKIN |
| | DSAVYYCARAGSYYYGSSYVEY | SLQPEDFGNYYCQHFWNT |
| | FDVWGTGTTVTVSS | PYTFGGGTKLEIK |
| 027 | SEQ ID NO: 38 | SEQ ID NO: 39 |
| | EVKILQSGGGLVQPGGSLKLSCA | DVLMTQTPLSLPVSLGDRA |
| | ASGIDFSRYWMSWVRRTPGKGL | SISCRSSQTIVHGNGHTSLE |
| | EWIGEINPDSSTINYAPSLKDKFII | WYLQKPGQSPQLLIYKVSN |
| | SRDNAKNTLYLQMSKVRSEDTA | RFSGVPDRFSASGSGTDFTL |
| | LYYCARLGIIPTYHYGSSGYYYA | TISRVEAEDLGVYYCFQVS |
| | MDYWGQGTSVTVSS | HVPYTFGGGTKLEIK |
| 029 | SEQ ID NO: 45 | SEQ ID NO: 46 |
| | DVKLVESGEDLVKPGGSLKLSC | DIVMTQSHKFMSTSVGDR |
| | AASGFTFSSYAMSWVRQTPEKR | VSITCKASQDVTTAVAWY |
| | LEWVAYVTSGGDYIYYADTVK | QQKPGQSPKLLIYSASYRY |
| | GRFTISRDDVRNTLYLQMSSLKS | TGVSDRFTGSGSGTDFTFTI |
| | EDTAMYYCTRVAYDYEWYFDV | SSVQAEDLALYYCQQHDSS |
| | WGTGAPVTVSS | PWTFGGGTKLEIK |
| 033 | SEQ ID NO: 52 | SEQ ID NO: 53 |
| | EVQLQQSGAELVRPGASVKLSC | DVVMTQIPLSLPVSLGDQA |
| | TASDFNIKDDYIHWVKQRPEQG | SISCRSSQSLVYSNGNTYLN |
| | LEWIGWIDLENGDTEYASKFQG | WYLQKPGQSPKLLIHKVSN |
| | KATITADTSSNTAYLQLSSLTSE | RFSGVPDRFSGSGSGTDFTL |
| | DTAVYYCTTKGSHWGQGTLVT | KISRVEAEDLGVYFCSQST |
| | VSA | HVPYTFGGGTKLEIR |
| 035 | SEQ ID NO: 59 | SEQ ID NO: 60 |
| | QVQLQQSGAELARPGASVKLSC | DIVLTQSPASLAVSLGQRA |
| | KASGYSFTSYGINWVKQRTGQG | TISCRASESVDNFGSSFLHW |
| | LEWIGEIYPRSGNTYYNEKFKDK | YQQKPGQAPKLLIYRASNL |
| | ATLTADKSSSTAYMELRSLTSED | ESGIPARFSGSGSRTDFTLTINPV |
| | SAVYFCARGGTYDGYYYAMDF | ETDDVATYYCQQVNK |
| | WGQGTSVTVSS | DPRTFGGGTKLEIK |
| 038 | SEQ ID NO: 66 | SEQ ID NO: 67 |
| | DVQVQESGPGLVKPSQSLSLTCS | DIVMTQSHKFMSASVGVR |
| | VTGYSITSGYYWNWIRQFPGNK | VSITCRASQDVDTAVAWY |
| | LEWMGYISYDGSNEYNLSLKNR | QQKPGQSPKLLIYWASTRH |
| | LSITRDTSKNQFFLKLNSVTTED | TGVPDRFTGSGSGTDFTLTI |
| | TATYYCAREEDGYYGGMDYWG | SNVQSEDLADYFCQQYSSY |
| | QGTSVTVSS | PYTFGGGTKLEIK |
| 039 | SEQ ID NO: 73 | SEQ ID NO: 74 |
| | EFQLQQSGPELVKPGASVKISCK | DVLMTQTPLFLPVSLGDQA |
| | ASGYSFTDYNLNWVRQSNGKSL | SISCRSSLNIVHSDGNTYLE |
| | EWIGLVDPIYGTIRYNQNFKDKA | WYLQKPGQSPKLLIYKVSN |
| | TLTVDQSSSTAYMQLNSLTSEDS | RFSGVPDRFSGSGSGTDFTL |
| | AVYYCASIMTAVRYFDHWGQG | KISGVEAEDLGVYYCFQGS |
| | TTLTVSS | HVPPTFGGGTKLEIK |
| 047 | SEQ ID NO: 79 | SEQ ID NO: 80 |
| | QVQLQQSGAELARPGASVKLSC | DIVLTQSPASLAVSLGQRA |
| | KASGYSFTSYGVNWVKQRTGQ | TISCRASESVDNFGSSFMH |
| | GLEWIGEIYPRSGNTYYNERFGD | WYQQKPGQAPKLLIYRAS |
| | KATLTADKSSSTAYMEIRSLTSE | NLESGIPARFSGSGSRTDFT |
| | DSAVYFCARGGTYDGYYYAMD | LTINPVETDDVATYYCQQV |
| | YWGQGTSVTVSS | NKDPRTFGGGTKLEIK |
| 048 | SEQ ID NO: 85 | SEQ ID NO: 86 |
| | EFQVQQSGPELVKPGASVKISCK | DVLMTQTPLSLPVSLGDQA |
| | ASGYSFSDYNMNWLKQSTGKSL | SISCRSSQNIVHTDGNTYLE |
| | EWIGLVDPIYGTIRYNQKFKGKA | WYLQKPGQSPKLLIYKVSN |
| | TLTVDQSSSTAYMQLNSLTSEDS | RFSGVPDRFSGSGSGTDFTL |
| | AVYYCALITTSVRYFDYWGQGT | KISRVEAEDLGVYYCFQGS |
| | TLTVSS | HVPPTFGGGTKLEIK |
| 057 | SEQ ID NO: 88 | SEQ ID NO: 89 |
| | QVQLQQSGAELARPGASVKLSC | NIVLTQSPASLAVSLGQRA |
| | KASGFSFTSYGINWVKQRTGQG | TISCRASESVDNFGSSFMH |
| | LEWIGEIYPRSGNTYYNEKLKGE | WYQQKPGQAPKLLIYRAS |
| | ATLTADRSSSTAYMELRSLTSED | NLESGIPARFGGSGSGTDFT |
| | SAVYFCARGGTYDGYYYAMDY | LTIDPVETDDVATYYCQQV |
| | WGQGTSVTVSS | NKDPRTFGGGTKLEIK |

TABLE 3
FR amino acid sequences of amino acid sequences of 13 antibodies

| Antibody | | FR1 | FR2 | FR3 | FR4 |
|---|---|---|---|---|---|
| 015 | HFR | SEQ ID NO: | SEQ ID NO: | SEQ ID NO: | SEQ ID NO: |
| | | 117 | 118 | 119 | 120 |
| | | | | KATLTVDKS | |
| | | EVQLQQSGP | WVKLSHGK | SSTAYMELN | WGQGTTLT |
| | | VLVKPGASV | SLEWIG | SLTSEDSAV | VSS |
| | | KLSCKASGF | | YYCAR | |
| | | TFT | | | |
| | LFR | SEQ ID NO: | SEQ ID NO: | SEQ ID NO: | SEQ ID NO: |
| | | 121 | 122 | 123 | 124 |
| | | DIQMTQSPS | WLQQEPDG | GVPKRFSGS | FGAGTKLEL |
| | | SLSASLGER | TIKRLIY | RSGSDYSLTI | K |
| | | VSLTC | | SSLDSEDFV | |
| | | | | VYYC | |
| 021 | HFR | SEQ ID NO: | SEQ ID NO: | SEQ ID NO: | SEQ ID NO: |
| | | 125 | 126 | 127 | 128 |
| | | DIQLQESGP | WIRQFPGNK | RISITRDTSK | WGQGTSVT |
| | | GLVKPSQSL | LEWMG | NQFFLKLNS | VSS |
| | | SLTCSVTGY | | VTTEDTATY | |
| | | SIT | | YCAR | |
| | LFR | SEQ ID NO: | SEQ ID NO: | SEQ ID NO: | SEQ ID NO: |
| | | 129 | 130 | 131 | 132 |
| | | DIVLTQSPAS | WYQQRPGQ | GIPARFSGSG | FGGGTKLEI |
| | | LAVSLGQRA | PPKLLIY | SRTDFTLTIN | K |
| | | TISC | | PVETDDVAT | |
| | | | | YYC | |
| 023 | HFR | SEQ ID NO: | SEQ ID NO: | SEQ ID NO: | SEQ ID NO: |
| | | 133 | 134 | 135 | 136 |
| | | QIQLVQSGP | WVKQAPGR | RFAFSLETSA | WGTGTTVT |
| | | VLKKPGETV | GFKWMG | STAYLQINN | VSS |
| | | KISCKASGY | | LKSEDTATY | |
| | | TFT | | FCVS | |
| | LFR | SEQ ID NO: | SEQ ID NO: | SEQ ID NO: | SEQ ID NO: |
| | | 137 | 138 | 139 | 124 |
| | | DIVMTQSHK | WYQQTPGQ | GVPDRFTGS | FGAGTKLEL |
| | | FMSTSVGDR | SPKLLIY | GSGTDFTFTI | K |
| | | VSITC | | SSVQAEDLA | |
| | | | | VYYC | |
| 024 | HFR | SEQ ID NO: | SEQ ID NO: | SEQ ID NO: | SEQ ID NO: |
| | | 140 | 141 | 142 | 136 |
| | | EVQLQQSGP | WVKQSHGK | KATLTVDKS | WGTGTTVT |
| | | ELVKPGASV | RLEWIG | SSSAYMDLR | VSS |
| | | KISCKASGY | | SLTSEDSAV | |
| | | TFT | | YYCAR | |
| | LFR | SEQ ID NO: | SEQ ID NO: | SEQ ID NO: | SEQ ID NO: |
| | | 143 | 144 | 145 | 132 |
| | | DIQMTQSPA | WYQQKQGA | GVPSRFSGS | FGGGTKLEI |
| | | SLSASVGET | SPQLLVY | GSGTEFSLKI | K |
| | | VTITC | | NSLQPEDFG | |
| | | | | NYYC | |
| 027 | HFR | SEQ ID NO: | SEQ ID NO: | SEQ ID NO: | SEQ ID NO: |
| | | 146 | 147 | 148 | 128 |
| | | EVKILQSGG | WVRRTPGK | KFIISRDNAK | WGQGTSVT |
| | | GLVQPGGSL | GLEWIG | NTLYLQMSK | VSS |
| | | KLSCAASGI | | VRSEDTALY | |
| | | DFS | | YCAR | |
| | LFR | SEQ ID NO: | SEQ ID NO: | SEQ ID NO: | SEQ ID NO: |
| | | 149 | 150 | 151 | 132 |
| | | DVLMTQTPL | WYLQKPGQ | GVPDRFSAS | FGGGTKLEI |
| | | SLPVSLGDR | SPQLLIY | GSGTDFTLTI | K |
| | | ASISC | | SRVEAEDLG | |
| | | | | VYYC | |
| 029 | HFR | SEQ ID NO: | SEQ ID NO: | SEQ ID NO: | SEQ ID NO: |
| | | 152 | 153 | 154 | 155 |
| | | DVKLVESGE | WVRQTPEK | RFTISRDDV | WGTGAPVT |
| | | DLVKPGGSL | RLEWVA | RNTLYLQMS | VSS |
| | | KLSCAASGF | | SLKSEDTAM | |
| | | TFS | | YYCTR | |
| | LFR | SEQ ID NO: | SEQ ID NO: | SEQ ID NO: | SEQ ID NO: |
| | | 137 | 156 | 157 | 132 |
| | | DIVMTQSHK | WYQQKPGQ | GVSDRFTGS | FGGGTKLEI |
| | | FMSTSVGDR | SPKLLIY | GSGTDFTFTI | K |
| | | VSITC | | SSVQAEDLA | |
| | | | | LYYC | |
| 033 | HFR | SEQ ID NO: | SEQ ID NO: | SEQ ID NO: | SEQ ID NO: |
| | | 158 | 159 | 160 | 16 |
| | | EVQLQQSGA | WVKQRPEQ | KATITADTSS | WGQGTLVT |
| | | ELVRPGASV | GLEWIG | NTAYLQLSS | VSA |
| | | KLSCTASDF | | LTSEDTAVY | |
| | | NIK | | YCTT | |
| | LFR | SEQ ID NO: | SEQ ID NO: | SEQ ID NO: | SEQ ID NO: |
| | | 162 | 163 | 164 | 165 |
| | | DVVMTQIPL | WYLQKPGQ | GVPDRFSGS | FGGGTKLEI |
| | | SLPVSLGDQ | SPKLLIH | GSGTDFTLKI | R |
| | | ASISC | | SRVEAEDLG | |
| | | | | VYFC | |
| 035 | HFR | SEQ ID NO: | SEQ ID NO: | SEQ ID NO: | SEQ ID NO: |
| | | 166 | 167 | 168 | 128 |
| | | QVQLQQSG | WVKQRTGQ | KATLTADKS | WGQGTSVT |
| | | AELARPGAS | GLEWIG | SSTAYMELR | VSS |
| | | VKLSCKASG | | SLTSEDSAV | |
| | | YSFT | | YFCAR | |
| | LFR | SEQ ID NO: | SEQ ID NO: | SEQ ID NO: | SEQ ID NO: |
| | | 129 | 169 | 131 | 132 |
| | | DIVLTQSPAS | WYQQKPGQ | GIPARFSGSG | FGGGTKLEI |
| | | LAVSLGQRA | APKLLIY | SRTDFTLTIN | K |
| | | TISC | | PVETDDVAT | |
| | | | | YYC | |
| 038 | HFR | SEQ ID NO: | SEQ ID NO: | SEQ ID NO: | SEQ ID NO: |
| | | 170 | 126 | 171 | 128 |
| | | DVQVQESGP | WIRQFPGNK | RLSITRDTSK | WGQGTSVT |
| | | GLVKPSQSL | LEWMG | NQFFLKLNS | VSS |
| | | SLTCSVTGY | | VTTEDTATY | |
| | | SIT | | YCAR | |
| | LFR | SEQ ID NO: | SEQ ID NO: | SEQ ID NO: | SEQ ID NO: |
| | | 172 | 156 | 173 | 132 |
| | | DIVMTQSHK | WYQQKPGQ | GVPDRFTGS | FGGGTKLEI |
| | | FMSASVGVR | SPKLLIY | GSGTDFTLTI | K |
| | | VSITC | | SNVQSEDLA | |
| | | | | DYFC | |
| 039 | HFR | SEQ ID NO: | SEQ ID NO: | SEQ ID NO: | SEQ ID NO: |
| | | 174 | 175 | 176 | 120 |
| | | EFQLQQSGP | WVRQSNGK | KATLTVDQS | WGQGTTLT |
| | | ELVKPGASV | SLEWIG | SSTAYMQLN | VSS |
| | | KISCKASGY | | SLTSEDSAV | |
| | | SFT | | YYCAS | |
| | LFR | SEQ ID NO: | SEQ ID NO: | SEQ ID NO: | SEQ ID NO: |
| | | 177 | 178 | 179 | 132 |
| | | DVLMTQTPL | WYLQKPGQ | GVPDRFSGS | FGGGTKLEI |
| | | FLPVSLGDQ | SPKLLIY | GSGTDFTLKI | K |
| | | ASISC | | SGVEAEDLG | |
| | | | | VYYC | |
| 047 | HFR | SEQ ID NO: | SEQ ID NO: | SEQ ID NO: | SEQ ID NO: |
| | | 166 | 167 | 168 | 128 |
| | | QVQLQQSG | WVKQRTGQ | KATLTADKS | WGQGTSVT |
| | | AELARPGAS | GLEWIG | SSTAYMEIR | VSS |
| | | VKLSCKASG | | SLTSEDSAV | |
| | | YSFT | | YFCAR | |
| | LFR | SEQ ID NO: | SEQ ID NO: | SEQ ID NO: | SEQ ID NO: |
| | | 129 | 169 | 131 | 132 |
| | | DIVLTQSPAS | WYQQKPGQ | GIPARFSGSG | FGGGTKLEI |
| | | LAVSLGQRA | APKLLIY | SRTDFTLTIN | K |
| | | TISC | | PVETDDVAT | |
| | | | | YYC | |
| 048 | HFR | SEQ ID NO: | SEQ ID NO: | SEQ ID NO: | SEQ ID NO: |
| | | 180 | 181 | 182 | 120 |
| | | EFQVQQSGP | WLKQSTGKS | KATLTVDQS | WGQGTTLT |
| | | ELVKPGASV | LEWIG | SSTAYMQLN | VSS |
| | | KISCKASGY | | SLTSEDSAV | |
| | | SFS | | YYCAL | |
| | LFR | SEQ ID NO: | SEQ ID NO: | SEQ ID NO: | SEQ ID NO: |
| | | 183 | 178 | 184 | 132 |
| | | DVLMTQTPL | WYLQKPGQ | GVPDRFSGS | FGGGTKLEI |
| | | SLPVSLGDQ | SPKLLIY | GSGTDFTLKI | K |
| | | ASISC | | SRVEAEDLG | |
| | | | | VYYC | |
| 057 | HFR | SEQ ID NO: | SEQ ID NO: | SEQ ID NO: | SEQ ID NO: |
| | | 185 | 167 | 186 | 128 |
| | | QVQLQQSG | WVKQRTGQ | EATLTADRS | WGQGTSVT |
| | | AELARPGAS | GLEWIG | SSTAYMELR | VSS |
| | | VKLSCKASG | | SLTSEDSAV | |
| | | FSFT | | YFCAR | |
| | LFR | SEQ ID NO: | SEQ ID NO: | SEQ ID NO: | SEQ ID NO: |
| | | 187 | 169 | 188 | 132 |
| | | NIVLTQSPAS | WYQQKPGQ | RASNLESGIP | FGGGTKLEI |
| | | LAVSLGQRA | APKLLIY | ARFGGSGSG | K |
| | | TISC | | TDFTLTIDPV | |
| | | | | ETDDVATYY | |
| | | | | C | |

Given that each of antibodies 015, 021, 023, 024, 027, 029, 033, 035, 038, 039, 047, 048 and 057 can bind to LAG3 and that antigen-binding specificity is provided primarily by the CDR1, CDR2 and CDR3 regions, the HCDR1, HCDR2 and HCDR3 sequences and LCDR1, LCDR2 and LCDR3 sequences of antibodies 015, 021, 023, 024, 027, 029, 033, 035, 038, 039, 047, 048 and 057 can be “mixed and matched” (i.e., CDRs from different antibodies can be mixed and matched, but each antibody must contain a HCDR1, HCDR2 and HCDR3 and a LCDR1, LCDR2 and LCDR3) to create anti-LAG3 binding molecules of the present disclosure. LAG3 binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence (s). Likewise, when VL CDR sequences are mixed and matched, the LCDR1, LCDR2 and/or LCDR3 sequence from a particular VL sequence preferably is replaced with a structurally similar CDR sequence (s). For example, the HCDR1s of antibodies 035, 047 and 057 share some structural similarity and therefore are amenable to mixing and matching. It will be readily apparent to a person skilled in the art that novel VH and VL sequences can be created by substituting one or more VH and/or VL CDR region sequences with structurally similar sequences from the CDR sequences disclosed herein for monoclonal antibodies 015, 021, 023, 024, 027, 029, 033, 035, 038, 039, 047, 048 and 057.

CDRs are known to be responsible for antigen binding. However, it has been found that not all of the 6 CDRs are indispensable or unchangeable. In other words, it is possible to replace or change or modify one or more CDRs in anti-LAG3 antibodies 015, 021, 023, 024, 027, 029, 033, 035, 038, 039, 047, 048 and 057, yet substantially retain the specific binding affinity to LAG3.

In certain embodiments, the antibodies and antigen-binding fragments thereof provided herein comprise suitable framework region (FR) sequences, as long as the antibodies and antigen-binding fragments thereof can specifically bind to LAG3. The CDR sequences provided in Table 1 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

Table 4 below shows the CDR amino acid sequences of 3 humanized antibodies for antibody 032, 4 humanized antibodies for antibody 029, and 12 humanized antibodies for antibody 023, which are designated as hu021.212, hu021.269, hu021.279, hu029.40, hu029.53, hu029.55, hu029.55.S93Q, hu023.04, hu023.05, hu023.06, hu023.07, hu023.08, hu023.09, hu023.10, hu023.11, hu023.12, hu023.13, hu023.14 and hu023.15. The CDR boundaries were defined or identified by the convention of Kabat. Table 5 below shows the heavy chain and light chain variable region amino acid sequences of humanized antibodies hu021.212, hu021.269, hu021.279, hu029.40, hu029.53, hu029.55, hu029.55.S93Q, hu023.04, hu023.05, hu023.06, hu023.07, hu023.08, hu023.09, hu023.10, hu023.11, hu023.12, hu023.13, hu023.14 and hu023.15. Table 6 below shows the FR amino acid sequences of humanized antibodies hu021.212, hu021.269, hu021.279, hu029.40, hu029.53, hu029.55, hu029.55.S93Q, hu023.04, hu023.05, hu023.06, hu023.07, hu023.08, hu023.09, hu023.10, hu023.11, hu023.12, hu023.13, hu023.14 and hu023.15.

TABLE 4
CDR amino acid sequences of humanized antibodies

| Antibody | | CDR1 | CDR2 | CDR3 |
|---|---|---|---|---|
| hu021.212 | HCDR | SEQ ID NO: 9 | SEQ ID NO: 90 | SEQ ID NO: 11 |
| | | SGYYWN | DISYEGGNNYNPSLKN | DITLTGAMDY |
| | LCDR | SEQ ID NO: 12 | SEQ ID NO: 91 | SEQ ID NO: 14 |
| | | RASESVDNYGI | RASNLET | QQSNKDPYT |
| | | SFMH | | |
| hu021.269 | HCDR | SEQ ID NO: 9 | SEQ ID NO: 10 | SEQ ID NO: 11 |
| | | SGYYWN | DISYDGGNNYNP | DITLTGAMDY |
| | | | SLKN | |
| | LCDR | SEQ ID NO: 12 | SEQ ID NO: 94 | SEQ ID NO: 14 |
| | | RASESVDNYGI | RASNRES | QQSNKDPYT |
| | | SFMH | | |
| hu021.279 | HCDR | SEQ ID NO: 97 | SEQ ID NO: 98 | SEQ ID NO: 11 |
| | | SGYYWT | DISYDAGNNYNPSLKN | DITLTGAMDY |
| | LCDR | SEQ ID NO: 12 | SEQ ID NO: 13 | SEQ ID NO: 14 |
| | | RASESVDNYGISFMH | RASNLES | QQSNKDPYT |
| hu029.40 | HCDR | SEQ ID NO: 40 | SEQ ID NO: 41 | SEQ ID NO: 42 |
| | | SYAMS | YVTSGGDYIYY | VAYDYEWYFDV |
| | | | ADTVKG | |
| | LCDR | SEQ ID NO: 43 | SEQ ID NO: 21 | SEQ ID NO: 44 |
| | | KASQDVTTAVA | SASYRYT | QQHDSSPWT |
| hu029.53 | HCDR | SEQ ID NO: 40 | SEQ ID NO: 41 | SEQ ID NO: 42 |
| | | SYAMS | YVTSGGDYIYY | VAYDYEWYFDV |
| | | | ADTVKG | |
| | LCDR | SEQ ID NO: 43 | SEQ ID NO: 21 | SEQ ID NO: 44 |
| | | KASQDVTTAVA | SASYRYT | QQHDSSPWT |
| hu029.55 | HCDR | SEQ ID NO: 40 | SEQ ID NO: 41 | SEQ ID NO: 42 |
| | | SYAMS | YVTSGGDYIYY | VAYDYEWYFDV |
| | | | ADTVKG | |
| | LCDR | SEQ ID NO: 43 | SEQ ID NO: 21 | SEQ ID NO: 44 |
| | | KASQDVTTAVA | SASYRYT | QQHDSSPWT |
| hu029.55. | HCDR | SEQ ID NO: 40 | SEQ ID NO: 41 | SEQ ID NO: 42 |
| S93Q | | SYAMS | YVTSGGDYIYY | VAYDYEWYFDV |
| | | | ADTVKG | |
| | LCDR | SEQ ID NO: 43 | SEQ ID NO: 21 | SEQ ID NO: 106 |
| | | KASQDVTTAVA | SASYRYT | QQHDQSPWT |
| hu023.04 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.05 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.06 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.07 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.08 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.09 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.10 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.11 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.12 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.13 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.14 | HCDR | SEQ ID NO: 17 | SEQ ID NO: 108 | SEQ ID NO: 19 |
| | | ESPMN | | |
| | | | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |
| hu023.15 | HCDR | SEQ ID NO: 17 | SEQ ID NO: 108 | SEQ ID NO: 19 |
| | | ESPMN | IIYTDTGEPTYAE | DYGSILYWYFD |
| | | | EFKG | V |
| | LCDR | SEQ ID NO: 20 | SEQ ID NO: 21 | SEQ ID NO: 109 |
| | | KASQDVSTAVA | SASYRYT | QQHYNAPPT |

TABLE 5
Variable region amino acid sequences of humanized antibodies

| Antibody | VH | VL |
|---|---|---|
| hu021.212 | SEQ ID NO: 92 | SEQ ID NO: 93 |
| | QVQLQESGPGLVKPSQTLSLT | EIVLTQSPATLSLSPGERATL |
| | CTVSGYSISSGYYWNWIRQHP | SCRASESVDNYGISFMHWY |
| | GNKLEWIGDISYEGGNNYNPS | QQKPGQAPRLLIYRASNLET |
| | LKNRVTISRDTSKNQFSLKLSSVT | GIPARFSGSGSRTDFTLTISSL |
| | AADTAVYYCARDITLTGA | EPEDFAVYYCQQSNKDPYTF |
| | MDYWGQGTTVTVSS | GQGTKLEIK |
| hu021.269 | SEQ ID NO: 95 | SEQ ID NO: 96 |
| | QVQLQESGPGLVKPSQTLSLT | EIVLTQSPATLSLSPGERATL |
| | CTVSGYSITSGYYWNWIRQHP | SCRASESVDNYGISFMHWY |
| | GNKLEWIGDISYDGGNNYNP | QQKPGQAPRLLIYRASNRES |
| | SLKNRVTISRDTSKNQFSLKLS | GIPARFSGSGSRTDFTLTISSL |
| | SVTAADTAVYYCARDITLTG | EPEDFAVYYCQQSNKDPYTF |
| | AMDYWGQGTTVTVSS | GQGTKLEIK |
| hu021.279 | SEQ ID NO: 99 | SEQ ID NO: 100 |
| | QIQLQESGPGLVKPGQTLSLT | EIVLTQSPATLSLSPGERATL |
| | CTVSGYSITSGYYWTWIRQHP | SCRASESVDNYGISFMHWY |
| | GNKLEWIGDISYDAGNNYNP | QQKPGQAPRLLIYRASNLES |
| | SLKNRITISRDTSKNQFSLKLS | GIPARFSGSGSGTDFTLTISSL |
| | SVTAEDTATYYCARDITLTGA | EPEDFAVYYCQQSNKDPYTF |
| | MDYWGQGTTVTVSS | GQGTKLEIK |
| hu029.40 | SEQ ID NO: 101 | SEQ ID NO: 102 |
| | QVQLVESGGGLVKPGGSLRL | DIQMTQSPSSVSTSVGDRVTI |
| | SCAASGFTFSSYAMSWVRQA | TCKASQDVTTAVAWYQQKP |
| | PGKRLEWVAYVTSGGDYIYY | GKAPKLLIYSASYRYTGVPS |
| | ADTVKGRFTISRDDAKNSLYL | RFSGSGSGTDFTFTISSVQPE |
| | QMNSLRAEDTAMYYCTRVA | |
| | YDYEWYFDVWGQGALVTVS | DFATYYCQQHDSSPWTFGQ |
| | S | GTKLEIK |
| hu029.53 | SEQ ID NO: 103 | SEQ ID NO: 104 |
| | QVQLVESGGGLVKPGGSLRL | DIQMTQSPSSVSASVGDRVTI |
| | SCAASGFTFSSYAMSWIRQAP | TCKASQDVTTAVAWYQQKP |
| | GKGLEWVAYVTSGGDYIYYA | GKAPKLLIYSASYRYTGVPS |
| | DTVKGRFTISRDDAKNSLYLQ | RFSGSGSGTDFTLTISSLQPE |
| | MNSLRAEDTAMYYCTRVAY | DFATYYCQQHDSSPWTFGQ |
| | DYEWYFDVWGQGALVTVSS | GTKLEIK |
| hu029.55 | SEQ ID NO: 105 | SEQ ID NO: 104 |
| | QVQLVESGGGLVKPGGSLRL | DIQMTQSPSSVSASVGDRVTI |
| | SCAASGFTFSSYAMSWIRQAP | TCKASQDVTTAVAWYQQKP |
| | GKGLEWVSYVTSGGDYIYYA | GKAPKLLIYSASYRYTGVPS |
| | DTVKGRFTISRDDAKNSLYLQ | RFSGSGSGTDFTLTISSLQPE |
| | MNSLRAEDTAVYYCTRVAYD | DFATYYCQQHDSSPWTFGQ |
| | YEWYFDVWGQGTLVTVSS | GTKLEIK |
| hu029.55.S93Q | SEQ ID NO: 105 | SEQ ID NO: 107 |
| | QVQLVESGGGLVKPGGSLRL | DIQMTQSPSSVSASVGDRVTI |
| | SCAASGFTFSSYAMSWIRQAP | TCKASQDVTTAVAWYQQKP |
| | GKGLEWVSYVTSGGDYIYYA | GKAPKLLIYSASYRYTGVPS |
| | DTVKGRFTISRDDAKNSLYLQ | RFSGSGSGTDFTLTISSLQPE |
| | MNSLRAEDTAVYYCTRVAYD | DFATYYCQQHDQSPWTFGQ |
| | YEWYFDVWGQGTLVTVSS | GTKLEIK |
| hu023.04 | SEQ ID NO: 110 | SEQ ID NO: 111 |
| | QVQLVQSGAEVKKPGASVKV | DIQMTQSPSSLSASVGDRVTI |
| | SCKASGYTFTESPMNWVRQA | TCKASQDVSTAVAWYQQKP |
| | PGQGFEWMGIIYTDTGEPTYA | GKAPKLLIYSASYRYTGVPS |
| | EEFKGRVTMTRDTSTSTVYM | RFSGSGSGTDFTLTISSLQPE |
| | ELSSLRSEDTAVYFCASDYGS | DFATYYCQQHYNAPPTFGQ |
| | ILYWYFDVWGQGTLVTVSS | GTKLEIK |
| hu023.05 | SEQ ID NO: 110 | SEQ ID NO: 112 |
| | QVQLVQSGAEVKKPGASVKV | DIQMTQSPSSLSASVGDRVTI |
| | SCKASGYTFTESPMNWVRQA | TCKASQDVSTAVAWYQQKP |
| | PGQGFEWMGIIYTDTGEPTYA | GKSPKLLIYSASYRYTGVPS |
| | EEFKGRVTMTRDTSTSTVYM | RFSGSGSGTDFTFTISSLQPE |
| | ELSSLRSEDTAVYFCASDYGS | DFATYYCQQHYNAPPTFGQ |
| | ILYWYFDVWGQGTLVTVSS | GTKLEIK |
| hu023.06 | SEQ ID NO: 110 | SEQ ID NO: 113 |
| | QVQLVQSGAEVKKPGASVKV | DIQMTQSPSSLSTSVGDRVTI |
| | SCKASGYTFTESPMNWVRQA | TCKASQDVSTAVAWYQQKP |
| | PGQGFEWMGIIYTDTGEPTYA | GKSPKLLIYSASYRYTGVPS |
| | EEFKGRVTMTRDTSTSTVYM | RFSGSGSGTDFTFTISSVQPE |
| | ELSSLRSEDTAVYFCASDYGS | DFATYYCQQHYNAPPTFGQ |
| | ILYWYFDVWGQGTLVTVSS | GTKLEIK |
| hu023.07 | SEQ ID NO: 114 | SEQ ID NO: 111 |
| | QVQLVQSGPEVKKPGASVKV | DIQMTQSPSSLSASVGDRVTI |
| | SCKASGYTFTESPMNWVRQA | TCKASQDVSTAVAWYQQKP |
| | PGQGFEWMGIIYTDTGEPTYA | GKAPKLLIYSASYRYTGVPS |
| | EEFKGRVTMTLDTSTSTVYM | RFSGSGSGTDFTLTISSLQPE |
| | ELSSLRSEDTAVYFCASDYGS | DFATYYCQQHYNAPPTFGQ |
| | ILYWYFDVWGQGTLVTVSS | GTKLEIK |
| hu023.08 | SEQ ID NO: 114 | SEQ ID NO: 112 |
| | QVQLVQSGPEVKKPGASVKV | DIQMTQSPSSLSASVGDRVTI |
| | SCKASGYTFTESPMNWVRQA | TCKASQDVSTAVAWYQQKP |
| | PGQGFEWMGIIYTDTGEPTYA | GKSPKLLIYSASYRYTGVPS |
| | EEFKGRVTMTLDTSTSTVYM | RFSGSGSGTDFTFTISSLQPE |
| | ELSSLRSEDTAVYFCASDYGS | DFATYYCQQHYNAPPTFGQ |
| | ILYWYFDVWGQGTLVTVSS | GTKLEIK |
| hu023.09 | SEQ ID NO: 114 | SEQ ID NO: 113 |
| | QVQLVQSGPEVKKPGASVKV | DIQMTQSPSSLSTSVGDRVTI |
| | SCKASGYTFTESPMNWVRQA | TCKASQDVSTAVAWYQQKP |
| | PGQGFEWMGIIYTDTGEPTYA | GKSPKLLIYSASYRYTGVPS |
| | EEFKGRVTMTLDTSTSTVYM | RFSGSGSGTDFTFTISSVQPE |
| | ELSSLRSEDTAVYFCASDYGS | DFATYYCQQHYNAPPTFGQ |
| | ILYWYFDVWGQGTLVTVSS | GTKLEIK |
| hu023.10 | SEQ ID NO: 115 | SEQ ID NO: 111 |
| | QVQLVQSGPELKKPGASVKV | DIQMTQSPSSLSASVGDRVTI |
| | SCKASGYTFTESPMNWVRQA | TCKASQDVSTAVAWYQQKP |
| | PGQGFEWMGIIYTDTGEPTYA | GKAPKLLIYSASYRYTGVPS |
| | EEFKGRFVFSLDTSVSTAYLQI | RFSGSGSGTDFTLTISSLQPE |
| | SSLKAEDTAVYFCVSDYGSIL | DFATYYCQQHYNAPPTFGQ |
| | YWYFDVWGQGTLVTVSS | GTKLEIK |
| hu023.11 | SEQ ID NO: 115 | SEQ ID NO: 112 |
| | QVQLVQSGPELKKPGASVKV | DIQMTQSPSSLSASVGDRVTI |
| | SCKASGYTFTESPMNWVRQA | TCKASQDVSTAVAWYQQKP |
| | PGQGFEWMGIIYTDTGEPTYA | GKSPKLLIYSASYRYTGVPS |
| | EEFKGRFVFSLDTSVSTAYLQI | RFSGSGSGTDFTFTISSLQPE |
| | SSLKAEDTAVYFCVSDYGSIL | DFATYYCQQHYNAPPTFGQ |
| | YWYFDVWGQGTLVTVSS | GTKLEIK |
| hu023.12 | SEQ ID NO: 115 | SEQ ID NO: 113 |
| | QVQLVQSGPELKKPGASVKV | DIQMTQSPSSLSTSVGDRVTI |
| | SCKASGYTFTESPMNWVRQA | TCKASQDVSTAVAWYQQKP |
| | PGQGFEWMGIIYTDTGEPTYA | GKSPKLLIYSASYRYTGVPS |
| | EEFKGRFVFSLDTSVSTAYLQI | RFSGSGSGTDFTFTISSVQPE |
| | SSLKAEDTAVYFCVSDYGSIL | DFATYYCQQHYNAPPTFGQ |
| | YWYFDVWGQGTLVTVSS | GTKLEIK |
| hu023.13 | SEQ ID NO: 116 | SEQ ID NO: 111 |
| | QIQLVQSGPELKKPGASVKIS | DIQMTQSPSSLSASVGDRVTI |
| | CKASGYTFTESPMNWVRQAP | TCKASQDVSTAVAWYQQKP |
| | GQGFEWMGIIYTDTGEPTYA | GKAPKLLIYSASYRYTGVPS |
| | EEFKGRFVFSLDTSVSTAYLQI | RFSGSGSGTDFTLTISSLQPE |
| | SSLKAEDTAVYFCVSDYGSIL | DFATYYCQQHYNAPPTFGQ |
| | YWYFDVWGQGTLVTVSS | GTKLEIK |
| hu023.14 | SEQ ID NO: 116 | SEQ ID NO: 112 |
| | QIQLVQSGPELKKPGASVKIS | DIQMTQSPSSLSASVGDRVTI |
| | CKASGYTFTESPMNWVRQAP | TCKASQDVSTAVAWYQQKP |
| | GQGFEWMGIIYTDTGEPTYA | GKSPKLLIYSASYRYTGVPS |
| | EEFKGRFVFSLDTSVSTAYLQI | RFSGSGSGTDFTFTISSLQPE |
| | SSLKAEDTAVYFCVSDYGSIL | DFATYYCQQHYNAPPTFGQ |
| | YWYFDVWGQGTLVTVSS | GTKLEIK |
| hu023.15 | SEQ ID NO: 116 | SEQ ID NO: 113 |
| | QIQLVQSGPELKKPGASVKIS | DIQMTQSPSSLSTSVGDRVTI |
| | CKASGYTFTESPMNWVRQAP | TCKASQDVSTAVAWYQQKP |
| | GQGFEWMGIIYTDTGEPTYA | GKSPKLLIYSASYRYTGVPS |
| | EEFKGRFVFSLDTSVSTAYLQI | RFSGSGSGTDFTFTISSVQPE |
| | SSLKAEDTAVYFCVSDYGSIL | DFATYYCQQHYNAPPTFGQ |
| | YWYFDVWGQGTLVTVSS | GTKLEIK |

TABLE 6
FR amino acid sequences of humanized antibodies

| Antibody | | FR1 | FR2 | FR3 | FR4 |
|---|---|---|---|---|---|
| hu021.212 | HFR | SEQ ID NO: 189 | SEQ ID NO: 190 | SEQ ID NO: 191 | SEQ ID NO: 192 |
| | | | | RVTISRDTSK | |
| | | QVQLQESGP | WIRQHPGNK | NQFSLKLSS | WGQGTTVT |
| | | GLVKPSQTL | LEWIG | VTAADTAV | VSS |
| | | SLTCTVSGY | | YYCAR | |
| | | SIS | | | |
| | LFR | SEQ ID NO: 193 | SEQ ID NO: 194 | SEQ ID NO: 195 | SEQ ID NO: 196 |
| | | EIVLTQSPAT | WYQQKPGQ | GIPARFSGSG | FGQGTKLEI |
| | | LSLSPGERA | APRLLIY | SRTDFTLTIS | K |
| | | TLSC | | SLEPEDFAV | |
| | | | | YYC | |
| hu021.269 | HFR | SEQ ID NO: 197 | SEQ ID NO: 190 | SEQ ID NO: 191 | SEQ ID NO: 192 |
| | | QVQLQESGP | WIRQHPGNK | RVTISRDTSK | WGQGTTVT |
| | | GLVKPSQTL | LEWIG | NQFSLKLSS | VSS |
| | | SLTCTVSGY | | VTAADTAV | |
| | | SIT | | YYCAR | |
| | LFR | SEQ ID NO: 193 | SEQ ID NO: 194 | SEQ ID NO: 195 | SEQ ID NO: 196 |
| | | EIVLTQSPAT | WYQQKPGQ | GIPARFSGSG | FGQGTKLEI |
| | | LSLSPGERA | APRLLIY | SRTDFTLTIS | K |
| | | TLSC | | SLEPEDFAV | |
| | | | | YYC | |
| hu021.279 | HFR | SEQ ID NO: 198 | SEQ ID NO: 190 | SEQ ID NO: 199 | SEQ ID NO: 192 |
| | | QIQLQESGP | WIRQHPGNK | RITISRDTSK | WGQGTTVT |
| | | GLVKPGQTL | LEWIG | NQFSLKLSS | VSS |
| | | SLTCTVSGY | | VTAEDTATY | |
| | | SIT | | YCAR | |
| | LFR | SEQ ID NO: 193 | SEQ ID NO: 194 | SEQ ID NO: 200 | SEQ ID NO: 196 |
| | | EIVLTQSPAT | WYQQKPGQ | GIPARFSGSG | FGQGTKLEI |
| | | LSLSPGERA | APRLLIY | SGTDFTLTIS | K |
| | | TLSC | | SLEPEDFAV | |
| | | | | YYC | |
| hu029.40 | HFR | SEQ ID NO: 201 | SEQ ID NO: 202 | SEQ ID NO: 203 | SEQ ID NO: 204 |
| | | QVQLVESGG | WVRQAPGK | RFTISRDDA | WGQGALVT |
| | | GLVKPGGSL | RLEWVA | KNSLYLQM | VSS |
| | | RLSCAASGF | | NSLRAEDTA | |
| | | TFS | | MYYCTR | |
| | LFR | SEQ ID NO: 205 | SEQ ID NO: 206 | SEQ ID NO: 207 | SEQ ID NO: 196 |
| | | DIQMTQSPS | WYQQKPGK | GVPSRFSGS | FGQGTKLEI |
| | | SVSTSVGDR | APKLLIY | GSGTDFTFTI | K |
| | | VTITC | | SSVQPEDFA | |
| | | | | TYYC | |
| hu029.53 | HFR | SEQ ID NO: 201 | SEQ ID NO: 208 | SEQ ID NO: 203 | SEQ ID NO: 204 |
| | | QVQLVESGG | WIRQAPGKG | RFTISRDDA | WGQGALVT |
| | | GLVKPGGSL | LEWVA | KNSLYLQM | VSS |
| | | RLSCAASGF | | NSLRAEDTA | |
| | | TFS | | MYYCTR | |
| | LFR | SEQ ID NO: 209 | SEQ ID NO: 206 | SEQ ID NO: 210 | SEQ ID NO: 196 |
| | | DIQMTQSPS | WYQQKPGK | GVPSRFSGS | FGQGTKLEI |
| | | SVSASVGDR | APKLLIY | GSGTDFTLTI | K |
| | | VTITC | | SSLQPEDFA | |
| | | | | TYYC | |
| hu029.55 | HFR | SEQ ID NO: 201 | SEQ ID NO: 211 | SEQ ID NO: 212 | SEQ ID NO: 213 |
| | | QVQLVESGG | WIRQAPGKG | RFTISRDDA | WGQGTLVT |
| | | GLVKPGGSL | LEWVS | KNSLYLQM | VSS |
| | | RLSCAASGF | | NSLRAEDTA | |
| | | TFS | | VYYCTR | |
| | LFR | SEQ ID NO: 209 | SEQ ID NO: 206 | SEQ ID NO: 210 | SEQ ID NO: 196 |
| | | DIQMTQSPS | WYQQKPGK | GVPSRFSGS | FGQGTKLEI |
| | | SVSASVGDR | APKLLIY | GSGTDFTLTI | K |
| | | VTITC | | SSLQPEDFA | |
| | | | | TYYC | |
| hu029.55.S93Q | HFR | SEQ ID NO: 201 | SEQ ID NO: 211 | SEQ ID NO: 212 | SEQ ID NO: 213 |
| | | QVQLVESGG | WIRQAPGKG | RFTISRDDA | WGQGTLVT |
| | | GLVKPGGSL | LEWVS | KNSLYLQM | VSS |
| | | RLSCAASGF | | NSLRAEDTA | |
| | | TFS | | VYYCTR | |
| | LFR | SEQ ID NO: 209 | SEQ ID NO: 206 | SEQ ID NO: 210 | SEQ ID NO: 196 |
| | | DIQMTQSPS | WYQQKPGK | GVPSRFSGS | FGQGTKLEI |
| | | SVSASVGDR | APKLLIY | GSGTDFTLTI | K |
| | | VTITC | | SSLQPEDFA | |
| | | | | TYYC | |
| hu023.04 | HFR | SEQ ID NO: 214 | SEQ ID NO: 215 | SEQ ID NO: 216 | SEQ ID NO: 213 |
| | | QVQLVQSG | WVRQAPGQ | RVTMTRDTS | WGQGTLVT |
| | | AEVKKPGAS | GFEWMG | TSTVYMELS | VSS |
| | | VKVSCKASG | | SLRSEDTAV | |
| | | YTFT | | YFCAS | |
| | LFR | SEQ ID NO: 217 | SEQ ID NO: 206 | SEQ ID NO: 210 | SEQ ID NO: 196 |
| | | DIQMTQSPS | WYQQKPGK | GVPSRFSGS | FGQGTKLEI |
| | | SLSASVGDR | APKLLIY | GSGTDFTLTI | K |
| | | VTITC | | SSLQPEDFA | |
| | | | | TYYC | |
| hu023.05 | HFR | SEQ ID NO: 214 | SEQ ID NO: 215 | SEQ ID NO: 216 | SEQ ID NO: 213 |
| | | QVQLVQSG | WVRQAPGQ | RVTMTRDTS | WGQGTLVT |
| | | AEVKKPGAS | GFEWMG | TSTVYMELS | VSS |
| | | VKVSCKASG | | SLRSEDTAV | |
| | | YTFT | | YFCAS | |
| | LFR | SEQ ID NO: 217 | SEQ ID NO: 218 | SEQ ID NO: 219 | SEQ ID NO: 196 |
| | | DIQMTQSPS | WYQQKPGK | GVPSRFSGS | FGQGTKLEI |
| | | SLSASVGDR | SPKLLIY | GSGTDFTFTI | K |
| | | VTITC | | SSLQPEDFA | |
| | | | | TYYC | |
| hu023.06 | HFR | SEQ ID NO: 214 | SEQ ID NO: 215 | SEQ ID NO: 216 | SEQ ID NO: 213 |
| | | QVQLVQSG | WVRQAPGQ | RVTMTRDTS | WGQGTLVT |
| | | AEVKKPGAS | GFEWMG | TSTVYMELS | VSS |
| | | VKVSCKASG | | SLRSEDTAV | |
| | | YTFT | | YFCAS | |
| | LFR | SEQ ID NO: 220 | SEQ ID NO: 218 | SEQ ID NO: 207 | SEQ ID NO: 196 |
| | | DIQMTQSPS | WYQQKPGK | GVPSRFSGS | FGQGTKLEI |
| | | SLSTSVGDR | SPKLLIY | GSGTDFTFTI | K |
| | | VTITC | | SSVQPEDFA | |
| | | | | TYYC | |
| hu023.07 | HFR | SEQ ID NO: 221 | SEQ ID NO: 215 | SEQ ID NO: 222 | SEQ ID NO: 213 |
| | | QVQLVQSGP | WVRQAPGQ | RVTMTLDTS | WGQGTLVT |
| | | EVKKPGASV | GFEWMG | TSTVYMELS | VSS |
| | | KVSCKASGY | | SLRSEDTAV | |
| | | TFT | | YFCAS | |
| | LFR | SEQ ID NO: 217 | SEQ ID NO: 206 | SEQ ID NO: 210 | SEQ ID NO: 196 |
| | | DIQMTQSPS | WYQQKPGK | GVPSRFSGS | FGQGTKLEI |
| | | SLSASVGDR | APKLLIY | GSGTDFTLTI | K |
| | | VTITC | | SSLQPEDFA | |
| | | | | TYYC | |
| hu023.08 | HFR | SEQ ID NO: 221 | SEQ ID NO: 215 | SEQ ID NO: 222 | SEQ ID NO: 213 |
| | | QVQLVQSGP | WVRQAPGQ | RVTMTLDTS | WGQGTLVT |
| | | EVKKPGASV | GFEWMG | TSTVYMELS | VSS |
| | | KVSCKASGY | | SLRSEDTAV | |
| | | TFT | | YFCAS | |
| | LFR | SEQ ID NO: 217 | SEQ ID NO: 218 | SEQ ID NO: 219 | SEQ ID NO: 196 |
| | | DIQMTQSPS | WYQQKPGK | GVPSRFSGS | FGQGTKLEI |
| | | SLSASVGDR | SPKLLIY | GSGTDFTFTI | K |
| | | VTITC | | SSLQPEDFA | |
| | | | | TYYC | |
| hu023.09 | HFR | SEQ ID NO: 221 | SEQ ID NO: 215 | SEQ ID NO: | SEQ ID NO: |
| | | QVQLVQSGP | WVRQAPGQ | RVTMTLDTS | WGQGTLVT |
| | | EVKKPGASV | GFEWMG | TSTVYMELS | VSS |
| | | KVSCKASGY | | SLRSEDTAV | |
| | | TFT | | YFCAS | |
| | LFR | SEQ ID NO: 220 | SEQ ID NO: 218 | SEQ ID NO: 207 | SEQ ID NO: 196 |
| | | DIQMTQSPS | WYQQKPGK | GVPSRFSGS | FGQGTKLEI |
| | | SLSTSVGDR | SPKLLIY | GSGTDFTFTI | K |
| | | VTITC | | SSVQPEDFA | |
| | | | | TYYC | |
| hu023.10 | HFR | SEQ ID NO: 223 | SEQ ID NO: 215 | SEQ ID NO: 224 | SEQ ID NO: 213 |
| | | QVQLVQSGP | WVRQAPGQ | RFVFSLDTS | WGQGTLVT |
| | | ELKKPGASV | GFEWMG | VSTAYLQISS | VSS |
| | | KVSCKASGY | | LKAEDTAVY | |
| | | TFT | | FCVS | |
| | LFR | SEQ ID NO: 217 | SEQ ID NO: 206 | SEQ ID NO: 210 | SEQ ID NO: 196 |
| | | DIQMTQSPS | WYQQKPGK | GVPSRFSGS | FGQGTKLEI |
| | | SLSASVGDR | APKLLIY | GSGTDFTLTI | K |
| | | VTITC | | SSLQPEDFA | |
| | | | | TYYC | |
| hu023.11 | HFR | SEQ ID NO: 223 | SEQ ID NO: 215 | SEQ ID NO: 224 | SEQ ID NO: 213 |
| | | QVQLVQSGP | WVRQAPGQ | RFVFSLDTS | WGQGTLVT |
| | | ELKKPGASV | GFEWMG | VSTAYLQISS | VSS |
| | | KVSCKASGY | | LKAEDTAVY | |
| | | TFT | | FCVS | |
| | LFR | SEQ ID NO: 217 | SEQ ID NO: 218 | SEQ ID NO: 219 | SEQ ID NO: 196 |
| | | DIQMTQSPS | WYQQKPGK | GVPSRFSGS | FGQGTKLEI |
| | | SLSASVGDR | SPKLLIY | GSGTDFTFTI | K |
| | | VTITC | | SSLQPEDFA | |
| | | | | TYYC | |
| hu023.12 | HFR | SEQ ID NO: 223 | SEQ ID NO: 215 | SEQ ID NO: 224 | SEQ ID NO: 213 |
| | | QVQLVQSGP | WVRQAPGQ | RFVFSLDTS | WGQGTLVT |
| | | ELKKPGASV | GFEWMG | VSTAYLQISS | VSS |
| | | KVSCKASGY | | LKAEDTAVY | |
| | | TFT | | FCVS | |
| | LFR | SEQ ID NO: 220 | SEQ ID NO: 218 | SEQ ID NO: 207 | SEQ ID NO: 196 |
| | | DIQMTQSPS | WYQQKPGK | GVPSRFSGS | FGQGTKLEI |
| | | SLSTSVGDR | SPKLLIY | GSGTDFTFTI | K |
| | | VTITC | | SSVQPEDFA | |
| | | | | TYYC | |
| hu023.13 | HFR | SEQ ID NO: 225 | SEQ ID NO: 215 | SEQ ID NO: 224 | SEQ ID NO: 213 |
| | | QIQLVQSGP | WVRQAPGQ | RFVFSLDTS | WGQGTLVT |
| | | ELKKPGASV | GFEWMG | VSTAYLQISS | VSS |
| | | KISCKASGY | | LKAEDTAVY | |
| | | TFT | | FCVS | |
| | LFR | SEQ ID NO: 217 | SEQ ID NO: 206 | SEQ ID NO: 210 | SEQ ID NO: 196 |
| | | DIQMTQSPS | WYQQKPGK | GVPSRFSGS | FGQGTKLEI |
| | | SLSASVGDR | APKLLIY | GSGTDFTLTI | K |
| | | VTITC | | SSLQPEDFA | |
| | | | | TYYC | |
| hu023.14 | HFR | SEQ ID NO: 225 | SEQ ID NO: 215 | SEQ ID NO: 224 | SEQ ID NO: 213 |
| | | QIQLVQSGP | WVRQAPGQ | RFVFSLDTS | WGQGTLVT |
| | | ELKKPGASV | GFEWMG | VSTAYLQISS | VSS |
| | | KISCKASGY | | LKAEDTAVY | |
| | | TFT | | FCVS | |
| | LFR | SEQ ID NO: 217 | SEQ ID NO: 218 | SEQ ID NO: 219 | SEQ ID NO: 196 |
| | | DIQMTQSPS | WYQQKPGK | GVPSRFSGS | FGQGTKLEI |
| | | SLSASVGDR | SPKLLIY | GSGTDFTFTI | K |
| | | VTITC | | SSLQPEDFA | |
| | | | | TYYC | |
| hu023.15 | HFR | SEQ ID NO: 225 | SEQ ID NO: 215 | SEQ ID NO: 224 | SEQ ID NO: 213 |
| | | QIQLVQSGP | WVRQAPGQ | RFVFSLDTS | WGQGTLVT |
| | | ELKKPGASV | GFEWMG | VSTAYLQISS | VSS |
| | | KISCKASGY | | LKAEDTAVY | |
| | | TFT | | FCVS | |
| | LFR | SEQ ID NO: 220 | SEQ ID NO: 218 | SEQ ID NO: 207 | SEQ ID NO: 196 |
| | | DIQMTQSPS | WYQQKPGK | GVPSRFSGS | FGQGTKLEI |
| | | SLSTSVGDR | SPKLLIY | GSGTDFTFTI | K |
| | | VTITC | | SSVQPEDFA | |
| | | | | TYYC | |

In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1-4, and/or light chain LFR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the present disclosure also provides humanized anti-LAG3 antibodies and antigen-binding fragments thereof comprising a heavy chain HFR1 comprising the sequence of QX₂₄QLQESGPGLVKPX₂₅QTLSLTCTVSGYSIX₂₆ (SEQ ID NO: 240), SEQ ID NO: 201, QX₃₉QLVQSGX₄₀EX₄₁KKPGASVKX₄₂SCKASGYTFT (SEQ ID NO: 248), SEQ ID NO: 117, 125, 133, 140, 146, 152, 158, 166, 170, 174, 180 or 185, or a homologous sequence of at least 85% sequence identity thereof, a heavy chain HFR2 comprising the sequence of SEQ ID NO: 190, WX₃1RQAPGKX₃₂LEWVX₃₃ (SEQ ID NO: 243), SEQ ID NO: 215, SEQ ID NO: 118, 126, 134, 141, 147, 153, 159, 167, 175 or 181, or a homologous sequence of at least 80% sequence identity thereof; a heavy chain HFR3 comprising the sequence of RX₂₇TISRDTSKNQFSLKLSSVTAX₂₈DTAX₂₉YYCAR (SEQ ID NO: 241), RFTISRDDAKNSLYLQMNSLRAEDTAX₃₄YYCTR (SEQ ID NO: 244), RX₄₃X₄₄X₄₅X₄₆X₄₇DTSX₄₈STX₄₉YX₅₀X₅₁X₅₂SSLX₅₃X₅₄EDTAVYFCX₅₅S (SEQ ID NO: 249), SEQ ID NO: 119, 127, 135, 142, 148, 154, 160, 168, 171, 176, 182 or 186, or a homologous sequence of at least 80% sequence identity thereof, and a heavy chain HFR4 comprising the sequence of SEQ ID NO: 192, WGQGX₃₅LVTVSS (SEQ ID NO: 245), SEQ ID NO: 213, SEQ ID NO: 120, 128, 136, 155 or 161, or a homologous sequence of at least 80% sequence identity thereof, wherein X₂₄ is V or I, X₂₅ is S or G, X₂₆ is S or T, X₂₇ is V or I, X₂₈ is A or E, X₂₉ is V or T, X₃₁ is V or I, X₃₂ is R or G, X₃₃ is A or S, X₃₄ is M or V, X₃₅ is A or T, X₃₉ is V or I, X₄₀ is A or P, X₄₁ is V or L, X₄₂ is V or I, X₄₃ is V or F, X₄₄ is T or V, X₄₅ is M or F, X₄₆ is T or S, X₄₇ is R or L, X₄₈ is T or V, X₄₉ is V or A, X₅₀ is M or L, X₅₁ is E or Q, X₅₂ is L or I, X₅₃ is R or K, X₅₄ is S or A, X₅₅ is A or V.

In certain embodiments, the present disclosure also provides humanized anti-LAG3 antibodies and antigen-binding fragments thereof comprising a light chain LFR1 comprising the sequence of SEQ ID NO: 193, DIQMTQSPSSVSX₃₆SVGDRVTITC (SEQ ID NO: 246), DIQMTQSPSSLSX₅₆SVGDRVTITC (SEQ ID NO: 250), SEQ ID NO: 121, 129, 137, 143, 149, 162, 172, 177, 183 or 187, or a homologous sequence of at least 80% sequence identity thereof, a light chain LFR2 comprising the sequence of SEQ ID NO: 194, SEQ ID NO: 206, WYQQKPGKX₅₇PKLLIY (SEQ ID NO: 251), SEQ ID NO: 122, 130, 138, 144, 150, 156, 163, 169 or 178, or a homologous sequence of at least 80% sequence identity thereof, a light chain LFR3 comprising the sequence of GIPARFSGSGSX₃₀TDFTLTISSLEPEDFAVYYC (SEQ ID NO: 242), GVPSRFSGSGSGTDFTX₃₇TISSX₃₈QPEDFATYYC (SEQ ID NO: 247), GVPSRFSGSGSGTDFTX₅₈TISSX₅₉QPEDFATYYC (SEQ ID NO: 252), SEQ ID NO: 123, 131, 139, 145, 151, 157, 164, 173, 179, 184 or 188, or a homologous sequence of at least 80% sequence identity thereof; and a light chain LFR4 comprising the sequence of SEQ ID NO: 196, SEQ ID NO: 124, 132 or 165, or a homologous sequence of at least 80% sequence identity thereof, wherein X₃₀ is R or G, X₃₆ is T or A, X₃₇ is L or F, X₃₈ is L or V, X₅₆ is T or A, X₅₇ is A or S, X₅₈ is L or F, X₅₉ is L or V.

In some embodiments, the HFR1 comprises an amino acid sequence of SEQ ID NO: 189; the HFR2 comprises an amino acid sequence of SEQ ID NO: 190; the HFR3 comprises an amino acid sequence of SEQ ID NO: 191; the HFR4 comprises an amino acid sequence of SEQ ID NO: 192; the LFR1 comprises an amino acid sequence of SEQ ID NO: 193; the LFR2 comprises an amino acid sequence of SEQ ID NO: 194; the LFR3 comprises an amino acid sequence of SEQ ID NO: 195; and the LFR4 comprises an amino acid sequence of SEQ ID NO: 196; or the HFR1 comprises an amino acid sequence of SEQ ID NO: 197; the HFR2 comprises an amino acid sequence of SEQ ID NO: 190; the HFR3 comprises an amino acid sequence of SEQ ID NO: 191; the HFR4 comprises an amino acid sequence of SEQ ID NO: 192; the LFR1 comprises an amino acid sequence of SEQ ID NO: 193; the LFR2 comprises an amino acid sequence of SEQ ID NO: 194; the LFR3 comprises an amino acid sequence of SEQ ID NO: 195; and the LFR4 comprises an amino acid sequence of SEQ ID NO: 196; or the HFR1 comprises an amino acid sequence of SEQ ID NO: 198; the HFR2 comprises an amino acid sequence of SEQ ID NO: 190; the HFR3 comprises an amino acid sequence of SEQ ID NO: 199; the HFR4 comprises an amino acid sequence of SEQ ID NO: 192; the LFR1 comprises an amino acid sequence of SEQ ID NO: 193; the LFR2 comprises an amino acid sequence of SEQ ID NO: 194; the LFR3 comprises an amino acid sequence of SEQ ID NO: 200; and the LFR4 comprises an amino acid sequence of SEQ ID NO: 196; or the HFR1 comprises an amino acid sequence of SEQ ID NO: 201; the HFR2 comprises an amino acid sequence of SEQ ID NO: 202; the HFR3 comprises an amino acid sequence of SEQ ID NO: 203; the HFR4 comprises an amino acid sequence of SEQ ID NO: 204; the LFR1 comprises an amino acid sequence of SEQ ID NO: 205; the LFR2 comprises an amino acid sequence of SEQ ID NO: 206; the LFR3 comprises an amino acid sequence of SEQ ID NO: 207; and the LFR4 comprises an amino acid sequence of SEQ ID NO: 196; or the HFR1 comprises an amino acid sequence of SEQ ID NO: 201; the HFR2 comprises an amino acid sequence of SEQ ID NO: 208; the HFR3 comprises an amino acid sequence of SEQ ID NO: 203; the HFR4 comprises an amino acid sequence of SEQ ID NO: 204; the LFR1 comprises an amino acid sequence of SEQ ID NO: 209; the LFR2 comprises an amino acid sequence of SEQ ID NO: 206; the LFR3 comprises an amino acid sequence of SEQ ID NO: 210; and the LFR4 comprises an amino acid sequence of SEQ ID NO: 196; or the HFR1 comprises an amino acid sequence of SEQ ID NO: 201; the HFR2 comprises an amino acid sequence of SEQ ID NO: 211; the HFR3 comprises an amino acid sequence of SEQ ID NO: 212; the HFR4 comprises an amino acid sequence of SEQ ID NO: 213; the LFR1 comprises an amino acid sequence of SEQ ID NO: 209; the LFR2 comprises an amino acid sequence of SEQ ID NO: 206; the LFR3 comprises an amino acid sequence of SEQ ID NO: 210; and the LFR4 comprises an amino acid sequence of SEQ ID NO: 196; or the HFR1 comprises an amino acid sequence of SEQ ID NO: 214; the HFR2 comprises an amino acid sequence of SEQ ID NO: 215; the HFR3 comprises an amino acid sequence of SEQ ID NO: 216; the HFR4 comprises an amino acid sequence of SEQ ID NO: 213; the LFR1 comprises an amino acid sequence of SEQ ID NO: 217; the LFR2 comprises an amino acid sequence of SEQ ID NO: 206; the LFR3 comprises an amino acid sequence of SEQ ID NO: 210; and the LFR4 comprises an amino acid sequence of SEQ ID NO: 196; or the HFR1 comprises an amino acid sequence of SEQ ID NO: 214; the HFR2 comprises an amino acid sequence of SEQ ID NO: 215; the HFR3 comprises an amino acid sequence of SEQ ID NO: 216; the HFR4 comprises an amino acid sequence of SEQ ID NO: 213; the LFR1 comprises an amino acid sequence of SEQ ID NO: 217; the LFR2 comprises an amino acid sequence of SEQ ID NO: 218; the LFR3 comprises an amino acid sequence of SEQ ID NO: 219; and the LFR4 comprises an amino acid sequence of SEQ ID NO: 196; or the HFR1 comprises an amino acid sequence of SEQ ID NO: 214; the HFR2 comprises an amino acid sequence of SEQ ID NO: 215; the HFR3 comprises an amino acid sequence of SEQ ID NO: 216; the HFR4 comprises an amino acid sequence of SEQ ID NO: 213; the LFR1 comprises an amino acid sequence of SEQ ID NO: 220; the LFR2 comprises an amino acid sequence of SEQ ID NO: 218; the LFR3 comprises an amino acid sequence of SEQ ID NO: 207; and the LFR4 comprises an amino acid sequence of SEQ ID NO: 196; or the HFR1 comprises an amino acid sequence of SEQ ID NO: 221; the HFR2 comprises an amino acid sequence of SEQ ID NO: 215; the HFR3 comprises an amino acid sequence of SEQ ID NO: 222; the HFR4 comprises an amino acid sequence of SEQ ID NO: 213; the LFR1 comprises an amino acid sequence of SEQ ID NO: 217; the LFR2 comprises an amino acid sequence of SEQ ID NO: 206; the LFR3 comprises an amino acid sequence of SEQ ID NO: 210; and the LFR4 comprises an amino acid sequence of SEQ ID NO: 196; or the HFR1 comprises an amino acid sequence of SEQ ID NO: 221; the HFR2 comprises an amino acid sequence of SEQ ID NO: 215; the HFR3 comprises an amino acid sequence of SEQ ID NO: 222; the HFR4 comprises an amino acid sequence of SEQ ID NO: 213; the LFR1 comprises an amino acid sequence of SEQ ID NO: 217; the LFR2 comprises an amino acid sequence of SEQ ID NO: 218; the LFR3 comprises an amino acid sequence of SEQ ID NO: 219; and the LFR4 comprises an amino acid sequence of SEQ ID NO: 196; or the HFR1 comprises an amino acid sequence of SEQ ID NO: 221; the HFR2 comprises an amino acid sequence of SEQ ID NO: 215; the HFR3 comprises an amino acid sequence of SEQ ID NO: 222; the HFR4 comprises an amino acid sequence of SEQ ID NO: 213; the LFR1 comprises an amino acid sequence of SEQ ID NO: 220; the LFR2 comprises an amino acid sequence of SEQ ID NO: 218; the LFR3 comprises an amino acid sequence of SEQ ID NO: 207; and the LFR4 comprises an amino acid sequence of SEQ ID NO: 196; or the HFR1 comprises an amino acid sequence of SEQ ID NO: 223; the HFR2 comprises an amino acid sequence of SEQ ID NO: 215; the HFR3 comprises an amino acid sequence of SEQ ID NO: 224; the HFR4 comprises an amino acid sequence of SEQ ID NO: 213; the LFR1 comprises an amino acid sequence of SEQ ID NO: 217; the LFR2 comprises an amino acid sequence of SEQ ID NO: 206; the LFR3 comprises an amino acid sequence of SEQ ID NO: 210; and the LFR4 comprises an amino acid sequence of SEQ ID NO: 196; or the HFR1 comprises an amino acid sequence of SEQ ID NO: 223; the HFR2 comprises an amino acid sequence of SEQ ID NO: 215; the HFR3 comprises an amino acid sequence of SEQ ID NO: 224; the HFR4 comprises an amino acid sequence of SEQ ID NO: 213; the LFR1 comprises an amino acid sequence of SEQ ID NO: 217; the LFR2 comprises an amino acid sequence of SEQ ID NO: 218; the LFR3 comprises an amino acid sequence of SEQ ID NO: 219; and the LFR4 comprises an amino acid sequence of SEQ ID NO: 196; or the HFR1 comprises an amino acid sequence of SEQ ID NO: 223; the HFR2 comprises an amino acid sequence of SEQ ID NO: 215; the HFR3 comprises an amino acid sequence of SEQ ID NO: 224; the HFR4 comprises an amino acid sequence of SEQ ID NO: 213; the LFR1 comprises an amino acid sequence of SEQ ID NO: 220; the LFR2 comprises an amino acid sequence of SEQ ID NO: 218; the LFR3 comprises an amino acid sequence of SEQ ID NO: 207; and the LFR4 comprises an amino acid sequence of SEQ ID NO: 196; or the HFR1 comprises an amino acid sequence of SEQ ID NO: 225; the HFR2 comprises an amino acid sequence of SEQ ID NO: 215; the HFR3 comprises an amino acid sequence of SEQ ID NO: 224; the HFR4 comprises an amino acid sequence of SEQ ID NO: 213; the LFR1 comprises an amino acid sequence of SEQ ID NO: 217; the LFR2 comprises an amino acid sequence of SEQ ID NO: 206; the LFR3 comprises an amino acid sequence of SEQ ID NO: 210; and the LFR4 comprises an amino acid sequence of SEQ ID NO: 196; or the HFR1 comprises an amino acid sequence of SEQ ID NO: 225; the HFR2 comprises an amino acid sequence of SEQ ID NO: 215; the HFR3 comprises an amino acid sequence of SEQ ID NO: 224; the HFR4 comprises an amino acid sequence of SEQ ID NO: 213; the LFR1 comprises an amino acid sequence of SEQ ID NO: 217; the LFR2 comprises an amino acid sequence of SEQ ID NO: 218; the LFR3 comprises an amino acid sequence of SEQ ID NO: 219; and the LFR4 comprises an amino acid sequence of SEQ ID NO: 196; or the HFR1 comprises an amino acid sequence of SEQ ID NO: 225; the HFR2 comprises an amino acid sequence of SEQ ID NO: 215; the HFR3 comprises an amino acid sequence of SEQ ID NO: 224; the HFR4 comprises an amino acid sequence of SEQ ID NO: 213; the LFR1 comprises an amino acid sequence of SEQ ID NO: 220; the LFR2 comprises an amino acid sequence of SEQ ID NO: 218; the LFR3 comprises an amino acid sequence of SEQ ID NO: 207; and the LFR4 comprises an amino acid sequence of SEQ ID NO: 196; or the HFR1 comprises an amino acid sequence of SEQ ID NO: 117; the HFR2 comprises an amino acid sequence of SEQ ID NO: 118; the HFR3 comprises an amino acid sequence of SEQ ID NO: 119; the HFR4 comprises an amino acid sequence of SEQ ID NO: 120; the LFR1 comprises an amino acid sequence of SEQ ID NO: 121; the LFR2 comprises an amino acid sequence of SEQ ID NO: 122; the LFR3 comprises an amino acid sequence of SEQ ID NO: 123; and the LFR4 comprises an amino acid sequence of SEQ ID NO: 124; or the HFR1 comprises an amino acid sequence of SEQ ID NO: 125; the HFR2 comprises an amino acid sequence of SEQ ID NO: 126; the HFR3 comprises an amino acid sequence of SEQ ID NO: 127; the HFR4 comprises an amino acid sequence of SEQ ID NO: 128; the LFR1 comprises an amino acid sequence of SEQ ID NO: 129; the LFR2 comprises an amino acid sequence of SEQ ID NO: 130; the LFR3 comprises an amino acid sequence of SEQ ID NO: 131; and the LFR4 comprises an amino acid sequence of SEQ ID NO: 132; or the HFR1 comprises an amino acid sequence of SEQ ID NO: 133; the HFR2 comprises an amino acid sequence of SEQ ID NO: 134; the HFR3 comprises an amino acid sequence of SEQ ID NO: 135; the HFR4 comprises an amino acid sequence of SEQ ID NO: 136; the LFR1 comprises an amino acid sequence of SEQ ID NO: 137; the LFR2 comprises an amino acid sequence of SEQ ID NO: 138; the LFR3 comprises an amino acid sequence of SEQ ID NO: 139; and the LFR4 comprises an amino acid sequence of SEQ ID NO: 124; or the HFR1 comprises an amino acid sequence of SEQ ID NO: 140; the HFR2 comprises an amino acid sequence of SEQ ID NO: 141; the HFR3 comprises an amino acid sequence of SEQ ID NO: 142; the HFR4 comprises an amino acid sequence of SEQ ID NO: 136; the LFR1 comprises an amino acid sequence of SEQ ID NO: 143; the LFR2 comprises an amino acid sequence of SEQ ID NO: 144; the LFR3 comprises an amino acid sequence of SEQ ID NO: 145; and the LFR4 comprises an amino acid sequence of SEQ ID NO: 132; or the HFR1 comprises an amino acid sequence of SEQ ID NO: 146; the HFR2 comprises an amino acid sequence of SEQ ID NO: 147; the HFR3 comprises an amino acid sequence of SEQ ID NO: 148; the HFR4 comprises an amino acid sequence of SEQ ID NO: 128; the LFR1 comprises an amino acid sequence of SEQ ID NO: 149; the LFR2 comprises an amino acid sequence of SEQ ID NO: 150; the LFR3 comprises an amino acid sequence of SEQ ID NO: 151; and the LFR4 comprises an amino acid sequence of SEQ ID NO: 132; or the HFR1 comprises an amino acid sequence of SEQ ID NO: 152; the HFR2 comprises an amino acid sequence of SEQ ID NO: 153; the HFR3 comprises an amino acid sequence of SEQ ID NO: 154; the HFR4 comprises an amino acid sequence of SEQ ID NO: 155; the LFR1 comprises an amino acid sequence of SEQ ID NO: 137; the LFR2 comprises an amino acid sequence of SEQ ID NO: 156; the LFR3 comprises an amino acid sequence of SEQ ID NO: 157; and the LFR4 comprises an amino acid sequence of SEQ ID NO: 132; or the HFR1 comprises an amino acid sequence of SEQ ID NO: 158; the HFR2 comprises an amino acid sequence of SEQ ID NO: 159; the HFR3 comprises an amino acid sequence of SEQ ID NO: 160; the HFR4 comprises an amino acid sequence of SEQ ID NO: 161; the LFR1 comprises an amino acid sequence of SEQ ID NO: 162; the LFR2 comprises an amino acid sequence of SEQ ID NO: 163; the LFR3 comprises an amino acid sequence of SEQ ID NO: 164; and the LFR4 comprises an amino acid sequence of SEQ ID NO: 165; or the HFR1 comprises an amino acid sequence of SEQ ID NO: 166; the HFR2 comprises an amino acid sequence of SEQ ID NO: 167; the HFR3 comprises an amino acid sequence of SEQ ID NO: 168; the HFR4 comprises an amino acid sequence of SEQ ID NO: 128; the LFR1 comprises an amino acid sequence of SEQ ID NO: 129; the LFR2 comprises an amino acid sequence of SEQ ID NO: 169; the LFR3 comprises an amino acid sequence of SEQ ID NO: 131; and the LFR4 comprises an amino acid sequence of SEQ ID NO: 132; or the HFR1 comprises an amino acid sequence of SEQ ID NO: 170; the HFR2 comprises an amino acid sequence of SEQ ID NO: 126; the HFR3 comprises an amino acid sequence of SEQ ID NO: 171; the HFR4 comprises an amino acid sequence of SEQ ID NO: 128; the LFR1 comprises an amino acid sequence of SEQ ID NO: 172; the LFR2 comprises an amino acid sequence of SEQ ID NO: 156; the LFR3 comprises an amino acid sequence of SEQ ID NO: 173; and the LFR4 comprises an amino acid sequence of SEQ ID NO: 132; or the HFR1 comprises an amino acid sequence of SEQ ID NO: 174; the HFR2 comprises an amino acid sequence of SEQ ID NO: 175; the HFR3 comprises an amino acid sequence of SEQ ID NO: 176; the HFR4 comprises an amino acid sequence of SEQ ID NO: 120; the LFR1 comprises an amino acid sequence of SEQ ID NO: 177; the LFR2 comprises an amino acid sequence of SEQ ID NO: 178; the LFR3 comprises an amino acid sequence of SEQ ID NO: 179; and the LFR4 comprises an amino acid sequence of SEQ ID NO: 132; or the HFR1 comprises an amino acid sequence of SEQ ID NO: 166; the HFR2 comprises an amino acid sequence of SEQ ID NO: 167; the HFR3 comprises an amino acid sequence of SEQ ID NO: 168; the HFR4 comprises an amino acid sequence of SEQ ID NO: 128; the LFR1 comprises an amino acid sequence of SEQ ID NO: 129; the LFR2 comprises an amino acid sequence of SEQ ID NO: 169; the LFR3 comprises an amino acid sequence of SEQ ID NO: 131; and the LFR4 comprises an amino acid sequence of SEQ ID NO: 132; or the HFR1 comprises an amino acid sequence of SEQ ID NO: 180; the HFR2 comprises an amino acid sequence of SEQ ID NO: 181; the HFR3 comprises an amino acid sequence of SEQ ID NO: 182; the HFR4 comprises an amino acid sequence of SEQ ID NO: 120; the LFR1 comprises an amino acid sequence of SEQ ID NO: 183; the LFR2 comprises an amino acid sequence of SEQ ID NO: 178; the LFR3 comprises an amino acid sequence of SEQ ID NO: 184; and the LFR4 comprises an amino acid sequence of SEQ ID NO: 132; or the HFR1 comprises an amino acid sequence of SEQ ID NO: 185; the HFR2 comprises an amino acid sequence of SEQ ID NO: 167; the HFR3 comprises an amino acid sequence of SEQ ID NO: 186; the HFR4 comprises an amino acid sequence of SEQ ID NO: 128; the LFR1 comprises an amino acid sequence of SEQ ID NO: 187; the LFR2 comprises an amino acid sequence of SEQ ID NO: 169; the LFR3 comprises an amino acid sequence of SEQ ID NO: 188; and the LFR4 comprises an amino acid sequence of SEQ ID NO: 132.

In certain embodiments, the present disclosure also provides humanized anti-LAG3 antibodies and antigen-binding fragments thereof comprising HFR1, HFR2, HFR3, and/or HFR4 sequences contained in a heavy chain variable region selected from a group consisting of: hu021.212-VH (SEQ ID NO: 92), hu021.269-VH (SEQ ID NO: 95), hu021.279-VH (SEQ ID NO: 99), hu029.40-VH (SEQ ID NO: 101), hu029.53-VH (SEQ ID NO: 103), hu029.55-VH/hu029.55.S93Q-VH (SEQ ID NO: 105), hu023.04-VH/hu023.05-VH/hu023.06-VH (SEQ ID NO: 110), hu023.07-VH/hu023.08-VH/hu023.09-VH (SEQ ID NO: 114), hu023.10-VH/hu023.11-VH/hu023.12-VH (SEQ ID NO: 115), and hu023.13-VH/hu023.14-VH/hu023.15-VH (SEQ ID NO: 116).

In certain embodiments, the present disclosure also provides humanized anti-LAG3 antibodies and antigen-binding fragments thereof comprising LFR1, LFR2, LFR3, and/or LFR4 sequences contained in a light chain variable region selected from a group consisting of: hu021.212-VL (SEQ ID NO: 93), hu021.269-VL (SEQ ID NO: 96), hu021.279-VL (SEQ ID NO: 100), hu029.40-VL (SEQ ID NO: 102), hu029.53-VL/hu029.55-VL (SEQ ID NO: 104), hu029.55.S93Q-VL (SEQ ID NO: 107), hu023.04-VL/hu023.07-VL/hu023.10-VL/hu023.13-VL (SEQ ID NO: 111), hu023.05-VL/hu023.08-VL/hu023.11-VL/hu023.14-VL (SEQ ID NO: 112), and hu023.06-VL/hu023.09-VL/hu023.12-VL/hu023.15-VL (SEQ ID NO: 113).

In certain embodiments, the humanized anti-LAG3 antibodies and antigen-binding fragments thereof provided herein comprise a heavy chain variable domain sequence selected from the group consisting of SEQ ID NO: 99 (hu021.279), SEQ ID NO: 105 (hu029.55 or hu029.55.S93Q), SEQ ID NO: 115 (hu023.11); and/or a light chain variable domain sequence selected from the group consisting of SEQ ID NO: 100 (hu021.279), SEQ ID NO: 107 (hu029.55.S93Q), SEQ ID NO: 112 (hu023.11).

The present disclosure also provides exemplary humanized antibodies of antibody 021, antibody 029 and antibody 023, respectively, including:

“hu021.212” comprising the heavy chain variable region of hu021.212-VH (SEQ ID NO: 92) and the light chain variable region of hu021.212-VL (SEQ ID NO: 93);

“hu021.269” comprising the heavy chain variable region of hu021.269-VH (SEQ ID NO: 95), and the light chain variable region of hu021.269-VL (SEQ ID NO: 96);

“hu021.279” comprising the heavy chain variable region of hu021.279-VH (SEQ ID NO: 99), and the light chain variable region of hu021.279-VL (SEQ ID NO: 100);

“hu029.40” comprising the heavy chain variable region of hu029.40-VH (SEQ ID NO: 101), and the light chain variable region of hu029.40-VL (SEQ ID NO: 102);

“hu029.53” comprising the heavy chain variable region of hu029.53-VH (SEQ ID NO: 103), and the light chain variable region of hu029.53-VL (SEQ ID NO: 104);

“hu029.55” comprising the heavy chain variable region of hu029.55-VH (SEQ ID NO: 105), and the light chain variable region of hu029.55-VL (SEQ ID NO: 104);

“hu029.55.S93Q” comprising the heavy chain variable region of hu029.55.S93Q-VH (SEQ ID NO: 105), and the light chain variable region of hu029.55.S93Q-VL (SEQ ID NO: 107);

“hu023.04” comprising the heavy chain variable region of hu023.04-VH (SEQ ID NO: 110), and the light chain variable region of hu023.04-VL (SEQ ID NO: 111).

“hu023.05” comprising the heavy chain variable region of hu023.05-VH (SEQ ID NO: 110), and the light chain variable region of hu023.05-VL (SEQ ID NO: 112).

“hu023.06” comprising the heavy chain variable region of hu023.06-VH (SEQ ID NO: 110), and the light chain variable region of hu023.06-VL (SEQ ID NO: 113).

“hu023.07” comprising the heavy chain variable region of hu023.07-VH (SEQ ID NO: 114), and the light chain variable region of hu023.07-VL (SEQ ID NO: 111).

“hu023.08” comprising the heavy chain variable region of hu023.08-VH (SEQ ID NO: 114), and the light chain variable region of hu023.08-VL (SEQ ID NO: 112).

“hu023.09” comprising the heavy chain variable region of hu023.09-VH (SEQ ID NO: 114), and the light chain variable region of hu023.09-VL (SEQ ID NO: 113).

“hu023.10” comprising the heavy chain variable region of hu023.10-VH (SEQ ID NO: 115), and the light chain variable region of hu023.10-VL (SEQ ID NO: 111).

“hu023.11” comprising the heavy chain variable region of hu023.11-VH (SEQ ID NO: 115), and the light chain variable region of hu023.11-VL (SEQ ID NO: 112).

“hu023.12” comprising the heavy chain variable region of hu023.12-VH (SEQ ID NO: 115), and the light chain variable region of hu023.12-VL (SEQ ID NO: 113).

“hu023.13” comprising the heavy chain variable region of hu023.13-VH (SEQ ID NO: 116), and the light chain variable region of hu023.13-VL (SEQ ID NO: 111).

“hu023.14” comprising the heavy chain variable region of hu023.14-VH (SEQ ID NO: 116), and the light chain variable region of hu023.14-VL (SEQ ID NO: 112).

“hu023.15” comprising the heavy chain variable region of hu023.15-VH (SEQ ID NO: 116), and the light chain variable region of hu023.15-VL (SEQ ID NO: 113).

These exemplary humanized anti-LAG3 antibodies retained the specific binding capacity or affinity to LAG3, and are at least comparable to, or even better than, the parent mouse antibody 021, 029 or 023 in that aspect. For example, data is provided in Example 5.

In some embodiments, the anti-LAG3 antibodies and antigen-binding fragments provided herein comprise all or a portion of the heavy chain variable domain and/or all or a portion of the light chain variable domain. In one embodiment, the anti-LAG3 antibody or an antigen-binding fragment thereof provided herein is a single domain antibody which consists of all or a portion of the heavy chain variable domain provided herein. More information of such a single domain antibody is available in the art (see, e.g. U.S. Pat. No. 6,248,516).

In certain embodiments, the anti-LAG3 antibodies or the antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-LAG3 antibodies or the antigen-binding fragments thereof provided herein comprises heavy chain constant regions of human IgG1, IgG2, IgG3, or IgG4. In certain embodiments, the light chain constant region comprises Cκ or Cλ. The constant region of the anti-LAG3 antibodies or the antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-LAG3 antibodies and antigen-binding fragments thereof provided herein comprises an Fc region of IgG1 or IgG3 isotype, which could induce ADCC or CDC; or alternatively, a constant region of IgG4 or IgG2 isotype, which has reduced or depleted effector function. In certain embodiments, the anti-LAG3 antibodies or antigen-binding fragments thereof provided herein comprise a wild type human IgG4 Fc region or other wild type human IgG4 alleles. In certain embodiments, the anti-LAG3 antibodies or antigen-binding fragments thereof provided herein comprise a human IgG4 Fc region comprising a S228P mutation. In some embodiments, the amino acid sequence of the S228P mutated constant region of IgG4 comprises the sequence of SEQ ID NO: 259.

In certain embodiments, the antibodies or the antigen-binding fragments thereof provided herein have a specific binding affinity to human LAG3 which is sufficient to provide for diagnostic and/or therapeutic use.

The antibodies or antigen-binding fragments thereof provided herein can be a monoclonal antibody, a polyclonal antibody, a humanized antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-LAG3 antibody or antigen-binding fragment thereof, which competes for binding to LAG3 with the antibody or antigen-binding fragment thereof provided herein. In certain embodiments, the present disclosure provides an anti-LAG3 antibody or antigen-binding fragment thereof, which competes for binding to human LAG3 with an antibody comprising a heavy chain variable region comprising the sequence of SEQ ID NO: 253, and a light chain variable region comprising the sequence of SEQ ID NO: 254 (BMS-986016). In certain embodiments, the present disclosure provides an anti-LAG3 antibody or antigen-binding fragment thereof, which competes for binding to human LAG3 with an antibody comprising a heavy chain variable region comprising the sequence of SEQ ID NO: 255, and a light chain variable region comprising the sequence of SEQ ID NO: 256 (BAP-050).

In certain embodiments, the present disclosure provides an anti-LAG3 antibody or antigen-binding fragment thereof, which competes for binding to human LAG3 with an antibody selected from the group consisting of: a) an antibody comprising a heavy chain variable region comprising the sequence of SEQ ID NO: 253, and a light chain variable region comprising the sequence of SEQ ID NO: 254; b) an antibody comprising a heavy chain variable region comprising the sequence of SEQ ID NO: 255, and a light chain variable region comprising the sequence of SEQ ID NO: 256, and wherein the antibody or an antigen-binding fragment thereof of is not any of BMS-986016 and BAP-050.

“BMS-986016” as used herein refers to an antibody or antigen binding fragment thereof comprising a heavy chain variable region having an amino acid sequence of SEQ ID NO: 253, and a light chain variable region having an amino acid sequence of SEQ ID NO: 254.

“BAP-050” as used herein refers to an antibody or antigen binding fragment thereof comprising a heavy chain variable region having an amino acid sequence of SEQ ID NO: 255, and a light chain variable region having an amino acid sequence of SEQ ID NO: 256.

### Antibody Variants

The antibodies and antigen-binding fragments thereof provided herein also encompass various variants of the antibody sequences provided herein.

In certain embodiments, the antibody variants comprise one or more modifications or substitutions in one or more of the CDR sequences as provided in Tables 1 and 3 above, one or more of the non-CDR sequences of the heavy chain variable region or light chain variable region provided in Tables 2 and 4 above, and/or the constant region (e.g. Fc region). Such variants retain binding specificity to LAG3 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g. charged residues such as Arg, Asp, His, Lys, and Glu) can be identified and replaced by a neutral or negatively charged amino acid (e.g. alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variants

Affinity variants of antibodies may contain modifications or substitutions in one or more CDR sequences as provided in Tables 1 and 3 above, one or more FR sequences as provided in Table 6 above, or the heavy or light chain variable region sequences provided in Tables 2 and 4 above. FR sequences can be readily identified by a person skilled in the art based on the CDR sequences in Tables 1 and 3 above and variable region sequences in Tables 2 and 4 above, as it is well-known in the art that a CDR region is flanked by two FR regions in the variable region. The affinity variants retain specific binding affinity to LAG3 of the parent antibody, or even have improved LAG3 specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Tables 1 and 3 above, and variable region sequences provided in Tables 2 and 4 above, one or more amino acid residues may be substituted yet the resulting antibody or antigen-binding fragment still retain the binding affinity or binding capacity to LAG3, or even have an improved binding affinity or capacity. Various methods known in the art can be used to achieve this purpose. For example, a library of antibody variants (such as Fab or scFv variants) can be generated and expressed with phage display technology, and then screened for the binding affinity to human LAG3. For another example, computer software can be used to virtually simulate the binding of the antibodies to human LAG3, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-LAG3 antibodies or antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Tables 1 and 3 above yet retaining the specific binding affinity to LAG3 at a level similar to or even higher than its parent antibody.

In certain embodiments, the anti-LAG3 antibodies or antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) listed in Tables 2 and 4 above yet retaining the specific binding affinity to LAG3 at a level similar to or even higher than its parent antibody. In some embodiments, a total of 1 to 10 amino acids have been substituted, inserted, or deleted in a variable region sequence listed in Tables 2 and 4 above. In some embodiments, the substitutions, insertions, or deletions occur in regions outside the CDRs (e.g. in the FRs).

### Glycosylation Variants

The anti-LAG3 antibodies or antigen-binding fragments thereof provided herein also encompass glycosylation variants, which can be obtained to either increase or decrease the extent of glycosylation of the antibodies or antigen binding fragments thereof.

The antibodies or antigen binding fragments thereof may comprise one or more modifications that introduce or remove a glycosylation site. A glycosylation site is an amino acid residue with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

In certain embodiments, the anti-LAG3 antibodies and antigen-binding fragments provided herein comprise a mutation at N297 (e.g. N297A, N297Q, or N297G) to remove the glycosylation site.

### Cysteine-Engineered Variants

The anti-LAG3 antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-LAG3 antibodies or antigen-binding fragments thereof provided herein also encompass Fc variants, which comprise one or more amino acid residue modifications or substitutions at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O,. et al., Mol Cancer Ther. 2008, 7(8): 2517-27; Shields R. L. et al., J. Biol. Chem, 2002, 277: 26733-26740; Shinkawa T. et al., J. Biol. Chem, 2003, 278: 3466-3473.

CDC activity of the antibodies or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821; and WO94/29351 concerning other examples of Fc region variants). One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-LAG3 antibodies or antigen-binding fragments thereof provided herein has reduced effector functions, and comprise one or more amino acid substitution(s) in IgG1 at a position selected from the group consisting of: 234, 235, 237, and 238, 268, 297, 309, 330, and 331. In certain embodiments, the anti-LAG3 antibodies or antigen-binding fragments thereof provided herein is of IgG1 isotype and comprise one or more amino acid substitution(s) selected from the group consisting of: N297A, N297Q, N297G, L235E, L234A, L235A, L234F, L235E, P331S, and any combination thereof. In certain embodiments, the anti-LAG3 antibodies or antigen-binding fragments thereof provided herein is of IgG2 isotype, and comprises one or more amino acid substitution(s) selected from the group consisting of: H268Q, V309L, A330S, P331S, V234A, G237A, P238S, H268A, and any combination thereof (e.g. H268Q/V309L/A330S/P331S, V234A/G237A/P238S/H268A/V309L/A330S/P331S). In certain embodiments, the anti-LAG3 antibodies or antigen-binding fragments thereof provided herein is of IgG4 isotype, and comprises one or more amino acid substitution(s) selected from the group consisting of: N297A, N297Q, N297G, L235E, L234A, L235A, and any combination thereof. In certain embodiments, the anti-LAG3 antibodies or antigen-binding fragments thereof provided herein is of IgG2/IgG4 cross isotype. Examples of IgG2/IgG4 cross isotype is described in Rother R P et al., Nat Biotechnol 25:1256-1264 (2007).

In certain embodiments, the anti-LAG3 antibodies and antigen-binding fragments provided herein is of IgG4 isotype and comprises one or more amino acid substitution(s) at one or more points of 228 and 235. In certain embodiments, the anti-LAG3 antibodies and antigen-binding fragments provided herein is of IgG4 isotype and comprises S228P mutation in the Fc region. In certain embodiments, the anti-LAG3 antibodies and antigen-binding fragments provided herein is of IgG4 isotype and comprises L235E mutation in the Fc region. In some embodiments, the amino acid sequence of the S228P mutated constant region of IgG4 comprises the sequence of SEQ ID NO: 259.

In certain embodiments, the anti-LAG3 antibodies or antigen-binding fragments thereof comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70: 3269-3277 (2010); and Hinton, P. et al., J. Immunology, 176:346-356 (2006).

In certain embodiments, anti-LAG3 antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g. as described in U.S. Pat. No. 5,731,168.

### Antigen-Binding FRAGMENTS

Provided herein are also anti-LAG3 antigen-binding fragments. Various types of antigen-binding fragments are known in the art and can be developed based on the anti-LAG3 antibodies provided herein, including for example, the exemplary antibodies whose CDRs are shown in Tables 1 and 3 above, and variable sequences are shown in Tables 2 and 4 above, and their different variants (such as affinity variants, glycosylation variants, Fc variants, cysteine-engineered variants and so on).

In certain embodiments, an anti-LAG3 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-LAG3 antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the anti-LAG3 antibodies or antigen-binding fragments thereof is bispecific. In certain embodiments, the antibody or antigen-binding fragment thereof is further linked to a second functional moiety having a different binding specificity from said LAG3 antibody, or antigen binding fragment thereof.

In certain embodiments, the bispecific antibodies or antigen-binding fragments thereof provided herein are capable of specifically binding to a second antigen other than LAG3, or a second epitope on LAG3. In certain embodiments, the second antigen is selected from the group consisting of CD19, CD20, CD22, CD24, CD25, CD30, CD33, CD38, CD44, CD52, CD56, CD70, CD96, CD97, CD99, CD123, CD279 (PD-1), CD274 (PD-L1), GPC-3, B7-H3, B7-H4, TROP2, CLDN18.2, EGFR, HER2, CD117, C-Met, PTHR2, and HAVCR2 (TIM3).

### Conjugates

In some embodiments, the anti-LAG3 antibodies or antigen-binding fragments thereof further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding, affinity binding, intercalation, coordinate binding, complexation, association, blending, or addition, among other methods. In some embodiments, the antibodies or antigen-binding fragments thereof can be linked to one or more conjugates via a linker.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g. a polymer such as PEG which extends half-life), a chemotherapeutic agent, a toxin, a radioactive isotope, a lanthanide, a detectable label (e.g. a luminescent label, a fluorescent label, an enzyme-substrate label), a DNA-alkylator, a topoisomerase inhibitor, a tubulin-binder, a purification moiety or other anticancer drugs.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), anti-mitotic agents (e.g. vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moieties, digoxigenin, biotin/avidin, DNA molecules or gold for detection.

In certain embodiments, the conjugate moiety can be a clearance-modifying agent which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibodies or antigen-binding fragments thereof provided herein is used as a base for a conjugate.

### Polynucleotides and Recombinant Methods

The present disclosure provides isolated polynucleotides that encode the anti-LAG3 antibodies or antigen-binding fragments thereof provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementarity sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The isolated polynucleotide that encodes the anti-LAG3 antibodies or antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-la), and a transcription termination sequence.

The present disclosure provides vectors comprising the isolated polynucleotide provided herein. In certain embodiments, the polynucleotide provided herein encodes the antibodies or antigen-binding fragments thereof, at least one promoter (e.g. SV40, CMV, EF-la) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS10, pLexA, pACT2.2, pCMV-SCRIPT.RTM., pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheriformis, Pseudomonas such as P. aeruginosa, and Streptomyces.

In addition to prokaryotes, eukaryotic microbes such as filamentous fungi or yeast are suitable cloning or expression hosts for anti-LAG3 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for anti-LAG3 antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-LAG3 antibodies or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g. from about 0-0.25M salt).

### Pharmaceutical Composition

The present disclosure further provides pharmaceutical compositions comprising the anti-LAG3 antibodies or antigen-binding fragments thereof and one or more pharmaceutically acceptable carriers.

The present disclosure further provides a pharmaceutical composition comprising the polynucleotides encoding the anti-LAG3 antibodies or antigen-binding fragments thereof, and one or more pharmaceutically acceptable carriers. Antibodies provided herein can also be produced in vivo by delivery of polynucleotides encoding the antibodies or antigen-binding fragments thereof provided herein, such as, for example, in-vitro-transcribed mRNA, or expression vectors. Methods are known in the art for polynucleotide delivery for antibody expression in vivo, see, for example, Rybakova, Y. et al, Molecular Therapy, vol. 27 (8), pp. 1415-1423 (2019); Deal, C. E. et al, Vaccines, 2021, 9, 108.

The present disclosure further provides pharmaceutical compositions comprising an expression vector comprising the polynucleotides encoding the anti-LAG3 antibodies or antigen-binding fragments thereof, and one or more pharmaceutically acceptable carriers.

In certain embodiments, the expression vector comprises a viral vector or a non-viral vector. Examples of viral vectors include, without limitation, adeno-associated virus (AAV) vector, lentivirus vector, retrovirus vector, and adenovirus vector. Examples of non-viral vectors include, without limitation, naked DNA, plasmid, exosome, mRNA, and so on. In certain embodiments, the expression vector is suitable for gene therapy in human. Suitable vectors for gene therapy include, for example, adeno-associated virus (AAV), or adenovirus vector. In certain embodiments, the expression vector comprises a DNA vector or a RNA vector. In certain embodiments, the pharmaceutically acceptable carriers are polymeric excipients, such as without limitation, microspheres, microcapsules, polymeric micelles and dendrimers. The polynucleotides, or polynucleotide vectors of the present disclosure may be encapsulated, adhered to, or coated on the polymer-based components by methods known in the art (see for example, W. Heiser, Nonviral gene transfer techniques, published by Humana Press, 2004; U.S. Pat. No. 6,025,337; Advanced Drug Delivery Reviews, 57(15): 2177-2202 (2005)).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-LAG3 antibody or antigen-binding fragment thereof or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the antibody or an antigen-binding fragment thereof provided herein. In certain embodiments, the present disclosure provides a kit comprising the antibody or an antigen-binding fragment thereof provided herein, and a second therapeutic agent. In certain embodiments, the second therapeutic agent is selected from the group consisting of a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

The present disclosure also provides methods of treating a LAG3 related disease, disorder or condition in a subject, comprising administering to the subject a therapeutically effective amount of the antibody or antigen-binding fragment thereof provided herein, or the polynucleotide encoding the antibody or antigen-binding fragment thereof provided herein and/or the pharmaceutical composition provided herein. In certain embodiments, the subject is human.

In some embodiments, the LAG3 related disease, disorder or condition is characterized in associated with a suppressed immune system in a subject.

In certain embodiments, the disease associated with a suppressed immune system is cancer or an infectious disease.

In some embodiments, the cancer is selected from the group consisting of an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

In some embodiments, the cancer is selected from the group consisting of colorectal cancer, hepatocellular carcinoma, glioma, kidney cancer, breast cancer, multiple myeloma, bladder cancer, neuroblastoma; sarcoma, nonHodgkin's lymphoma, non-small cell lung cancer, ovarian cancer, pancreatic cancer and rectal cancer.

In some embodiments, the cancer is colon cancer or breast cancer.

In some embodiments, the disease associated with a suppressed immune system is a pathogen-associated disease. In some embodiments, the pathogen-associated disease is selected from the group consisting of chronic viral, bacterial, fungal and parasitic infections.

In some embodiments, the chronic viral infections include infections by Epstein Barr virus, Hepatitis A Virus (HAV); Hepatitis B Virus (HBV); Hepatitis C Virus (HCV); herpes viruses (e.g. HSV-1, HSV-2, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia Zambia, trichinella, Dientamoeba Fragilis, Trypanosoma brucei, Trypanosoma cruzi, and Leishmania donovani.

In certain embodiments, the LAG3 related disease, disorder or condition is characterized in up-regulation of LAG3 in a subject, wherein expressing of LAG3 in a cell in the subject is at a level significantly higher than that would have been expected of a normal cell. The presence and/or amount of LAG3 in an interested biological sample can be indicative of whether the subject from whom the biological sample is derived could likely respond to an anti-LAG3 antibody. Various methods can be used to determine the presence and/or amount of LAG3 in a test biological sample from the subject. For example, the test biological sample can be exposed to anti-LAG3 antibody or antigen-binding fragment thereof, which binds to and detects the expressed LAG3 protein. Alternatively, LAG3 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. In certain embodiments, presence or up-regulated level of the LAG3 in the test biological sample indicates likelihood of responsiveness. The term “up-regulated” as used herein, refers to an overall increase of no less than 10%, 15%, 20%, 25%, 30%, 35%, 40%, 45%, 50%, 55%, 60%, 65%, 70%, 75%, 80% or greater, in the expression level of LAG3 in the test sample, as compared to the LAG3 expression level in a reference sample as detected using the same method. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor).

In another aspect, methods are provided to treat a disease, disorder or condition in a subject that would benefit from modulation of LAG3 activity, comprising administering a therapeutically effective amount of the antibody or antigen-binding fragment thereof provided herein and/or the pharmaceutical composition provided herein to a subject in need thereof. In certain embodiments, the disease or condition is a LAG3 related disease, disorder or condition.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g. physician or veterinarian) as indicated by these and other circumstances or requirements.

In certain embodiments, the antibody or antigen-binding fragment provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g. a therapeutic response). For example, a single dose may be administered, or several divided doses may be administered over time.

The antibodies or antigen-binding fragments thereof provided herein may be administered by any route known in the art, such as for example parenteral (e.g. subcutaneous, intraperitoneal, intravenous, including intravenous infusion, intramuscular, or intradermal injection) or non-parenteral (e.g. oral, intranasal, intraocular, sublingual, rectal, or topical) routes.

In some embodiments, the antibodies or antigen-binding fragments thereof provided herein may be administered alone or in combination a therapeutically effective amount of a second therapeutic agent. For example, the antibodies or antigen-binding fragments thereof disclosed herein may be administered in combination with a second therapeutic agent, for example, a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, or cytokines.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, an antibody or antigen-binding fragment thereof provided herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition. However, an antibody or antigen-binding fragment thereof administered “in combination” with another therapeutic agent does not have to be administered simultaneously with or in the same composition as the agent. An antibody or antigen-binding fragment thereof administered prior to or after another agent is considered to be administered “in combination” with that agent as the phrase is used herein, even if the antibody or antigen-binding fragment and the second agent are administered via different routes. Where possible, additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In some embodiments, the additional therapeutic agent is a chemotherapeutic agent, which may be administered with anti-LAG3 antibody as described herein and the, include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolamide), ethylenimines/methylmel amine such as thriethylenemel amine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-merca.rho.topurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza) and release of transcriptional suppression (ATRA) therapies can also be combined with the antigen binding proteins. In one embodiment the chemotherapeutic agent is selected from the group consisting of taxanes (like e.g. paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio). In one embodiment, the additional chemotherapeutic agent is selected from 5-fluorouracil (5-FU), leucovorin, irinotecan, or oxaliplatin. In one embodiment the chemotherapeutic agent is 5-fluorouracil, leucovorin and irinotecan (FOLFIRI). In one embodiment the chemotherapeutic agent is 5-fluorouracil, and oxaliplatin (FOLFOX).

In another aspect, the present disclosure provides methods of detecting the presence or amount of LAG3 in a sample, comprising contacting the sample with the antibody or antigen-binding fragment thereof provided herein, and determining the presence or the amount of LAG3 in the sample.

In another aspect, the present disclosure provides a method of selecting a patient with a disease, disorder or condition associated with a suppressed immune system, comprising the steps of: a) contacting a sample obtained from the subject with the antibody or an antigen-binding fragment thereof of the present disclosure; b) determining the presence or amount of LAG3 in the sample; and c) correlating the presence or the amount of LAG3 to existence or status of the LAG3 related disease, disorder or condition in the subject.

In another aspect, the present disclosure provides kits comprising the antibody or antigen-binding fragment thereof provided herein, optionally conjugated with a detectable moiety, which is useful in detecting a LAG3 related disease, disorder or condition. The kits may further comprise instructions for use.

In another aspect, the present disclosure also provides use of the antibody or antigen-binding fragment thereof provided herein in the manufacture of a medicament for treating, preventing or alleviating a LAG3 related disease, disorder or condition in a subject, in the manufacture of a diagnostic reagent for diagnosing a LAG3 related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Reagent Generation

### 1.1. Reference Antibody Generation

The DNA sequences encoding variable regions of anti-LAG3 reference antibodies BMS-986016 (see WO 2015/116539 A1, SEQ ID NO:3 and 5), BAP-050 (see US20150259420A1, SEQ ID NO:6 and 16) and TSR-033 (see WO 2018/201096 A1, SEQ ID NO:21 and 22) were cloned into the vectors expressing human IgG constant regions. The variable region amino acid sequences of reference antibodies BMS-986016, BAP-050, and TSR-033 are shown in Table 7 as below. The expression plasmids transfected Expi293 cells (Invitrogen) were cultured at 37° C. for 5 days. Then the culture medium was collected and centrifuged to remove cell pellets. The harvested supernatant was purified using Protein A affinity chromatography column.

TABLE 7
Variable region amino acid sequences of 3 reference antibodies

| Antibody | VH | VL |
|---|---|---|
| BMS- | QVQLQQWGAGLLKPSETLSLTCAV | EIVLTQSPATLSLSPGERATLSCRA |
| 986016 | YGGSFSDYYWNWIRQPPGKGLEWI | SQSISSYLAWYQQKPGQAPRLLI |
| | GEINHRGSTNSNPSLKSRVTLSLDTS | YDASNRATGIPARFSGSGSGTDFT |
| | KNQFSLKLRSVTAADTAVYYCAFG | LTISSLEPEDFAVYYCQQRSNWPL |
| | YSDYEYNWFDPWGQGTLVTVSS | TFGQGTNLEIK (SEQ ID NO: 254) |
| | (SEQ ID NO: 253) | |
| BAP-050 | QIQLVQSGPELKKPGETVKISCKASG | DIQMTQTTSSLSASLGDRVTISCS |
| | FTLTNYGMNWVRQTPGKGLKWMG | SSQDISNYLNWYQQKPDGTVKV |
| | WINTDTGEPTYADDFKGRFAFSLET | LIYYTSTLHLGVPSRFSGSGSGTD |
| | SASTASLQINNLKNADTATYFCARN | YSLTISNLELEDIATYYCQQYYNL |
| | PPYYYGTNNAEAMDYWGQGTAVT | PWTFGGGTKLEIK (SEQ ID NO: |
| | VSS (SEQ ID NO: 255) | 256) |
| TSR-033 | EVQLVQSGAEVKKPGATVKISCKAS | DIVMTQTPLSLSVTPGQPASISCR |
| | GFSIKDDYIHWVQQAPGKGLEWMG | SSQSLVHSDSNTYLHWYLQKPG |
| | WIDAMNDDSQYSSKFQGRVTITVD | QSPQLLIYLVSNRFSGVPDRFSGS |
| | TSTNTAYMKLSSLRSEDTAVYYCTY | GSGTDFTLKISRVEAEDVGVYFC |
| | AFGGYWGQGTTVTVSS (SEQ ID NO: | GQSTHVPYAFGGGTKVEIK (SEQ |
| | 257) | ID NO: 258) |

### 1.2. LAG3 Stable Expression Cell Lines Generation

The DNA sequences encoding full length human LAG3 (NP_002277.4), cyno LAG3 (XP_005570011. 1), and mouse LAG3 (NP_032505.1) were cloned into the pMRES vector (Clontech) respectively. 293F cells (Invitrogen) transfected with human LAG3 or cyno LAG3 expression plasmid were selectively cultured in medium containing 0.5 μg/ml puromycin for 2 weeks. BW5147 mouse T lymphoma cells transfected with human LAG3 expression plasmid were selectively cultured in medium containing 1 μg/ml puromycin for 2 weeks. CHOK1 cells (Invitrogen) transfected with human LGA3 or mouse LAG3 expression plasmid were selectively cultured in medium containing 6 μg/ml puromycin for 2 weeks. Then single cell clones stably expressing human LAG3, cyno LAG3 or mouse LAG3 were isolated by limiting dilution and screened by FACS.

Another human LAG3 (NP_002277.4) expression plasmid cloned with pCMV3 vector was purchased from Sino Biological (HG16498-UT). In a similar way, 293F cells and CHOK1 cells transfected with human LAG3 expression plasmid from Sino Biological were selectively cultured in medium containing 400 μg/ml or 800 μg/ml hygromycin for 2 weeks. Then single cell clones stably expressing human LAG3 were isolated by limiting dilution and screened by FACS.

### 1.3. Recombinant Proteins Generation

Recombinant proteins of human IgG Fc (hFc) tagged human LAG3 extracellular domain (ECD, L23-L450) and human LAG3 D1-D2 (L23-G261) were generated by Chempartner. Recombinant proteins of 6×His tagged and mouse human IgG Fc (mFc) tagged human LAG3 ECD were purchased from Biointron. 6×His tagged LSEctin ECD (S54-C293, NP_940894) recombinant protein was purchased from R&D system (2947-CL). 6×His tagged FGL1 recombinant protein was purchased from Sino Biological.

### Example 2. Antibody Generation

### 2.1. Preparation of Immunogen for Protein Immunization

hFc tagged human LAG3 D1-D2 recombinant protein was used as immunogen for protein immunization (refer to Example 1.3).

### 2.2. Preparation of Immunogen for Cell Immunization

BW5147 mouse T lymphoma cells stably expressing human LAG3 were used as immunogen for cell immunization (refer to Example 1.2).

### 2.3. Immunization

SJL/J mice (SLAC) were immunized by two different strategies of protein immunization using human LAG3 D1-D2 recombinant protein and cell immunization using BW5147 mouse T lymphoma cells stably expressing human LAG3. ELISA assay with human LAG3 ECD recombinant protein and FACS assay with 293F cells stably expressing human LAG3 were used to detect serum titer of immunized mice. Mice with high serum titer were selected for hybridoma fusion.

### 2.4. Hybridoma Generation

5 days after final boost, mice were sacrificed and the spleen cells were collected. 1% (v/v) NH₄OH was added to lyse erythrocytes. Then the washed spleen cells were fused with SP2/0 mouse myeloma cells (ATCC) by high-efficiency electro-fusion or PEG method. After cell fusion, the fused cells were seeded into 96-well plates at the density of 2×10⁴ cells/well with 200 [l DMEM medium containing 20% FBS and 1% HAT.

### 2.5. Hybridoma Screening

10-12 days after fusion, fusion plates were primarily screened by ELISA assay with human LAG3 D1-D2 recombinant protein or Acumen assay (TTP Labtech) with CHOK1 cells stably expressing human LAG3. The hybridoma cells from positive wells were amplified into 24-well plates for 2^{nd} screening. In 2^{nd} screening, binding activity was assessed by ELISA assay with human LAG3 D1-D2 recombinant protein and FACS assay with 293F cells stably expressing human LAG3. In addition, the species cross reactivity, LAG3 and MHC II interaction blocking activity were also detected in 2^{nd} screening for hybridoma characterization (refer to Example 3 for methods of the characterization assays). Then the wells of hybridoma cells with top binding and blocking activities were selected for subcloning.

### 2.6. Hybridoma Subcloning

Hybridoma cells from selected wells were limited diluted into 96-well plates followed by same screening as described in Example 2.5. Then the monoclonal hybridoma cell lines with top binding and blocking activities were cryopreserved as hit clones.

A total of 13 clones were identified as hit clones, and the hybridoma antibodies purified from these clones were assigned as 015, 021, 023, 024, 027, 029, 033, 035, 038, 039, 047, 048 and 057 respectively.

### Example 3. Antibody Characterization

### 3.1. Hybridoma Antibody Production and Purification

After about 14 days of culturing, the hybridoma cell culture medium was collected and centrifuged to remove cells. After filtered through 0.22 m PES membrane and adjusting pH to 7.4, the harvested supernatants were loaded to Protein A affinity chromatography column (GE). Antibodies were eluted by 0.1 M citrate sodium buffer (pH3.0) followed by immediately neutralization using Tris buffer (pH8.0). After dialysis with PBS buffer, the antibody concentration was determined by Nano Drop (Thermo Fisher). The purity of proteins was evaluated by SDS-PAGE and HPLC-SEC (Agilent). The endotoxin level was detected with Endochrome-K kit (Charles River).

### 3.2 Binding Activity Detection

Binding activity of the purified hybridoma antibodies against human LAG3 was detected by ELISA assay using hFc tagged human LAG3 D1-D2 recombinant protein. Briefly the antibodies were incubated with ELISA microplate coated antigens at 37° C. for 1 hour. After washing, horseradish peroxidase (HRP) labeled anti-human IgG 2^{nd} Ab (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read and EC₅₀ was calculated using GraphPad Prism9.0. As summarized in Table 8, all antibodies as tested strongly bind to human LAG3.

| | FACS | | | | | NFAT | Antigen | |
|---|---|---|---|---|---|---|---|---|
| | 293F-human LAG3 | 293F-cyno LAG3 | ELISA (EC₅₀, nM) | LAG3/MHC II interaction blocking | | reporter | specific T | Epitope |
| Ab Code | (EC₅₀, nM) | (MFI at 20 nM) | hLAG3 D1-D2 | IC₅₀ (nM) | TOP blocking (%) | assay | cell Assay | Group |
| 015 | 2.09 | no binding | 0.25 | 2.7 | 94.3 | + | +++ | III |
| 021 | 5.52 | 104.7 | 0.02 | 4.0 | 98.6 | + | ++ | II |
| 023 | 2.65 | 125.4 | 0.05 | 4.9 | 84.2 | +++ | ++ | I |
| 024 | 2.04 | 154.3 | 0.05 | 5.5 | 81.0 | + | ++ | I |
| 027 | 2.38 | 132.3 | 0.05 | 5.4 | 97.2 | + | ++ | II |
| 029 | 4.58 | 35.6 | 0.06 | 4.6 | 90.7 | ++ | + | III |
| 033 | 2.33 | 83.4 | 0.07 | 4.8 | 98.8 | ++ | + | I |
| 035 | 0.66 | 155.4 | 0.06 | 5.0 | 99.2 | ++ | ++ | II |
| 038 | 1.12 | 142.2 | 0.07 | 4.2 | 100 | +++ | ++ | I |
| 039 | 1.38 | no binding | 0.04 | 5.2 | 94.9 | +++ | ++ | III |
| 047 | 1.14 | 128.9 | 0.04 | 7.7 | 82.7 | ++ | ++ | II |
| 048 | 1.19 | no binding | 0.05 | 7.6 | 92.9 | +++ | ++ | I |
| 057 | 1.04 | 83.4 | 0.02 | 5.1 | 87.6 | ++ | + | II |

Plus symbol stands for the tested antibody can block LAG3/MHC II mediated inhibitory of reporter gene activity or antigen specific T cell activation. Number of plus symbol is used to indicate relative activity level (+++ > ++ > +)

### 3.3. Species Cross Reactivity Detection

Species cross reactivity of the purified hybridoma antibodies against human, cyno and mouse LAG3 was determined by FACS assay using 293F cells stably expressing human LAG3, cyno LAG3 or CHOK1 cells stably expressing mouse LAG3. Briefly the antibodies were incubated with 2×10⁵ target cells at 4° C. for 1 hour. After washing, fluorescence labeled anti-mouse IgG 2^{nd} antibody (Life Technologies) was added and incubated at 4° C. for 1 hour. Geometric median fluorescence intensity (MFI) was detected and EC₅₀ was calculated using GraphPad Prism9.0. The species cross reactivity property of 13 functional antibodies is summarized in Table 8. Other than 015, 039 and 048, all antibodies as tested can bind to cyno LAG3. None of tested antibodies can bind to mouse LAG3.

### 3.5. LAG3/MIIC II Interaction Blocking Activity Detection

Competitive FACS assay was used to determine whether the purified hybridoma antibodies can block LAG3 and MHC II interaction. Briefly antibody and hFc tagged human LAG3 ECD recombinant protein were co-incubated with Raji cells endogenously expressing MHC II. After washing, dye labeled anti-human Fc 2^{nd} antibody (Sigma) was added and incubated at 37° C. for 1 hour. Fluorescence intensity was detected. Blocking ratio was determined by blockade of human LAG3 ECD recombinant protein binding to Raji cells. IC₅₀ and top blocking ratio calculated using GraphPad Prism9.0 are summarized in Table 8. All antibodies as tested can block LAG3 and MHC II interaction.

### 3.6. NFAT Reporter Assay

Blocking activity of the purified hybridoma antibodies to relieve LAG3/MHC II mediated suppression was assessed by cell based NFAT reporter assay. Briefly each tested antibody was mixed with 25 ul Jurkat cells (4×10⁶/ml) stably expressing human LAG3 and NFAT reporter gene. Simultaneously a Raji cell suspension (1×10⁶/ml) was prepared with 100 ng/ml SED toxin. Added 25 ul SED loaded Raji cells to the mixture of Jurkat cells and tested antibodies followed by incubation at 37° C. for 24 hours. Luciferase activity was measured using One Glo Promega reagent and plate reader. The blocking activity was determined by reporter activity fold change compared to isotype control. As summarized in Table 8, all antibodies as tested can block LAG3/MHC II mediated inhibition of reporter gene activity at different levels.

### 3.7. Antigen Specific T Cell Activation Assay

Functional activity of the purified hybridoma antibodies to relieve LAG3/MHC II mediated suppression was assessed by T cell activation assay that utilized an antigen specific mouse T cell hybridoma (3E4). Hybridoma 3E4 expresses a T cell receptor (TCR) specific for a peptide from OVA (323-339) and secretes IL-2 when co-cultured with peptide-pulsed, MHC-matched, antigen presenting cells. Since human LAG3 is capable of binding to mouse MHC Class II, expression of human LAG3 in 3E4 could exert an inhibitory effect through engagement with MHC Class II on the murine splenocytes. This inhibition can be reversed by LAG3 blocking antibody. Briefly each tested antibody was mixed with 50 ul 3E4 cells (2×10⁶/ml) stably expressing human LAG3. Simultaneously a mouse splenocytes suspension (4×10⁶/ml) was prepared with 10 μg/ml OVA peptide. Added 50 ul OVA peptide loaded mouse splenocytes to the mixture of 3E4 cells and tested antibodies followed by incubation at 37° C. for 40 hours. mIL-2 was measured by Mouse IL-2 DuoSet ELISA (R&D SYSTEM) and plate reader. The function activity was determined by increase of IL-2 secretion. As summarized in Table 8, all antibodies as tested can block LAG3/MHC II inhibited antigen-pulsed IL-2 secretion of T cells at different levels.

### 3.8. Epitope Binning

Competitive ELISA assay was used for epitope binning of the purified hybridoma antibodies. Briefly excessive competitor antibody and biotin labelled hFc tagged human LAG3 ECD recombinant protein were co-incubated with ELISA microplate coated BMS-986016 or BAP-050. After washing, HRP labeled anti-streptavidin (SA) 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Competition ratio was calculated. The antibodies that can compete each other to bind to LAG3 may have related binding epitopes. As shown in Table 8 and Table 9, a total of 13 anti-LAG3 antibodies were divided into 3 epitope groups based on competition profile. 033, 024, 023, 048, and 038 showed more than 30% competition with BMS-986016 but less than 30% competition with BAP-050, which were grouped into epitope group I. 029, 039, and 015 showed less than 30% competition with both BMS-986016 and BAP-050, which were grouped into epitope group III. 021, 027, 047, 057 and 035 showed less than 30% competition with BMS-986016 but more than 30% competition with BAP-050, which were grouped into epitope group II.

TABLE 9
Epitope binning summary of anti-LAG3 hybridoma antibodies
% competition

Coating

| Competitor | BMS-986016 | BAP-050 |
|---|---|---|
| 033 | 92.5 | 3.8 |
| 024 | 73.0 | −0.5 |
| 023 | 43.7 | −2.7 |
| 048 | 41.7 | 10.0 |
| 038 | 33.5 | 6.4 |
| 029 | 20.3 | 23.5 |
| 039 | 19.3 | 2.6 |
| 015 | 10.0 | 1.6 |
| 021 | 6.9 | 57.1 |
| 027 | 7.2 | 85.1 |
| 047 | 8.9 | 87.4 |
| 057 | 29.3 | 87.7 |
| 035 | 21.7 | 91.9 |

### 3.9. Hybridoma Sequencing

Total RNA isolated from monoclonal hybridoma cells was reverse-transcribed into cDNA using either isotype-specific anti-sense primers or universal primers following the technical manual of SMARTScribe Reverse Transcriptase. Then the cDNA was used as template to amplify antibody fragments of heavy chain and light chain according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes and insert fragments were analyzed by DNA sequencing. Finally, the consensus sequences were identified as antibody variable regions of heavy chain and light chain.

### Example 4. Chimeric Antibody Generation and Characterization

### 4.1. Chimeric Antibody Generation and Production

According to hybridoma sequencing results mouse anti-LAG3 functional hits were converted into human IgG4 chimeric antibodies with S228P mutation for characterization. Briefly the DNA sequence encoding heavy chain variable region was cloned into the pcDNA3.4-hIgG4P vector (Biointron) carrying human IgG4 heavy chain constant region with S228P mutation, wherein the amino acid sequence of the heavy chain constant region is shown as SEQ ID NO: 259.

Amino acid sequence of human IgG4 heavy chain constant region with S228P mutation (SEQ ID NO: 259):

ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

The DNA sequence encoding light chain variable region was cloned into the pcDNA3.4-hIgGk vector (Biointron) carrying human kappa light chain constant region. Expi293 cells (Life Technologies) co-transfected with antibody heavy and light chain expression plasmids were expanded at 37° C. for 5 days. Then the culture medium was collected and centrifuged to remove cells. The harvested supernatants were loaded to Protein A affinity chromatography column. Antibodies were eluted by 0.04 M citrate sodium buffer (pH3.1) followed by immediately neutralization using Tris buffer (pH8.0). After dialysis with PBS buffer, the antibody concentration was determined by Nano Drop (ThermoFisher). The purity of proteins was evaluated by SDS-PAGE and HPLC-SEC (Shimadzu). The endotoxin level was detected with Kinetic Turbidimetric LAL Assay. The resulting chimeric antibodies are referred to herein as 015c, 021c, 023c, 024c, 027c, 029c, 033c, 035c, 038c, 039c, 047c, 048c and 057c respectively where the suffix “c” indicates chimeric.

### 4.2. Chimeric Antibody Characterization

### 4.2.1. Binding Activity and Species Cross Reactivity Detection

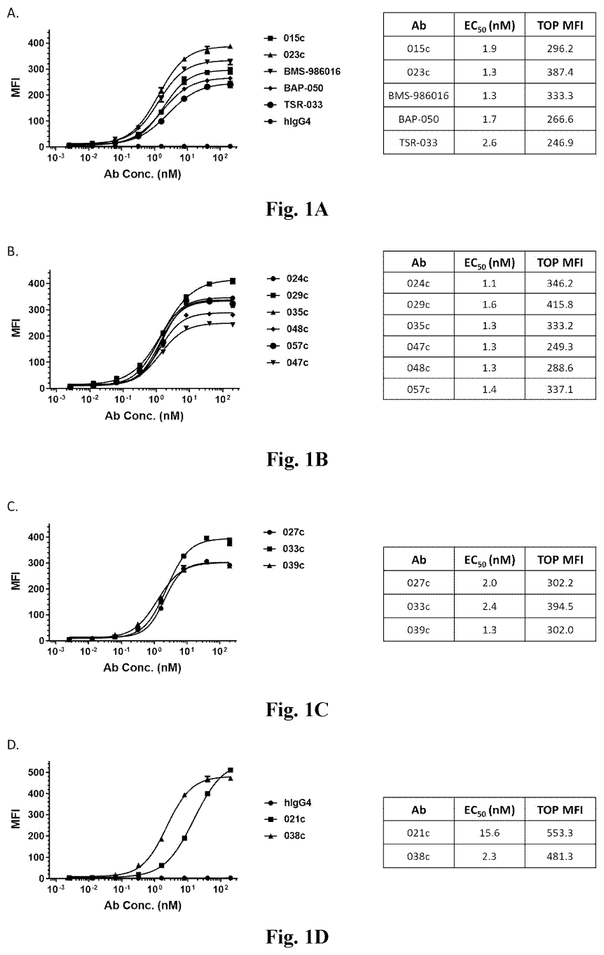

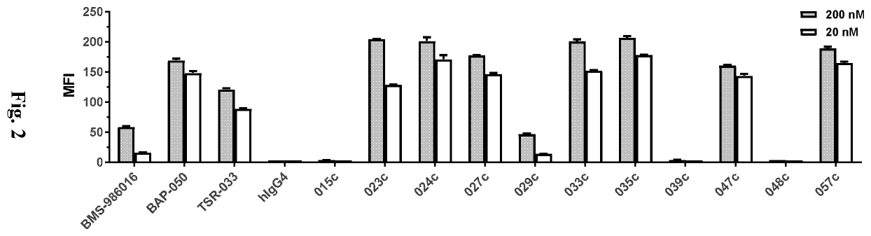

Binding activity and species cross reactivity of the purified chimeric antibodies against human and cyno LAG3 were determined by FACS assay (refer to Example 3.3) using 293F cells stably expressing human LAG3 (FIGS. 1A, 1B, 1C and 1D) or cyno LAG3 (FIG. 2). MFI was detected and EC₅₀ was calculated using GraphPad Prism9.0. As shown in FIGS. 1A, 1B, 1C and 1D, all antibodies as tested strongly bind to human LAG3. As shown in FIG. 2, other than 015c, 039c and 048c, all antibodies as tested can bind to cyno LAG3, which is consistent with the results obtained by hybridoma antibodies.

### 4.2.2. LAG3/MHC II Interaction Blocking Activity Detection

Activity of the purified chimeric antibodies to block LAG3 and MHC II interaction was determined by competitive FACS assay. Briefly antibody and mFc tagged human LAG3 ECD recombinant protein were co-incubated with Raji cells endogenously expressing MHC II. After washing, dye labeled anti-mouse Fc 2^{nd} antibody (Sigma) was added and incubated at 37° C. for 1 hour. Fluorescence intensity was detected. Blocking ratio was determined by blockade of human LAG3 ECD recombinant protein binding to Raji cells. IC₅₀ and top blocking ratio calculated using GraphPad Prism9.0 are summarized in Table 10. All antibodies as tested can block LAG3 and MHC II interaction.

TABLE 10
LAG3/MHC II blocking activity summary
of anti-LAG3 chimeric antibodies

LAG3/MHC II interaction blocking

| Ab Code | IC₅₀ (nM) | TOP blocking (%) |
|---|---|---|
| 015c | 5.4 | 77.1 |
| 021c | 4.2 | 99.9 |
| 023c | 3.7 | 94.6 |
| 024c | 3.7 | 96.9 |
| 027c | 4.5 | 91.2 |
| 029c | 4.2 | 96.4 |
| 033c | 3.9 | 95.0 |
| 035c | 4.3 | 95.9 |
| 038c | 5.2 | 98.4 |
| 039c | 4.7 | 62.0 |
| 047c | 3.6 | 90.4 |
| 048c | 4.0 | 64.6 |
| 057c | 2.5 | 95.1 |
| BMS-986016 | 4.6 | 92.7 |
| TSR-033 | 12.4 | 96.0 |

### 4.2.3. LAG3/LSECtin Interaction Blocking Activity Detection

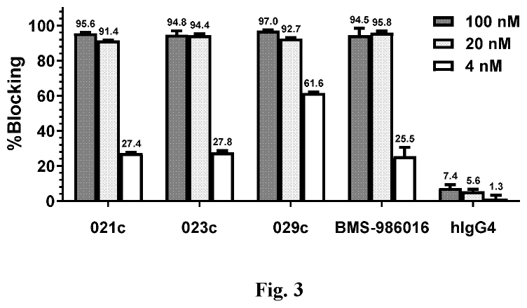

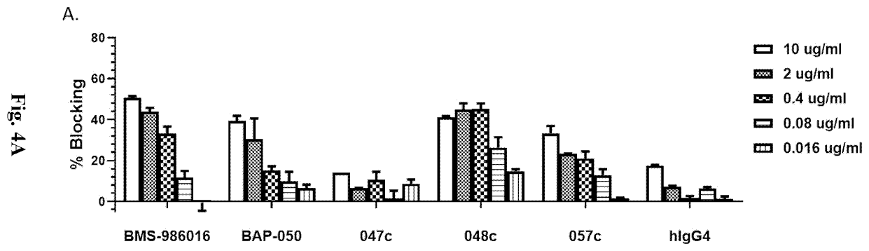

Activity of the purified chimeric antibodies to block LAG3 and LSECtin interaction was determined by competitive ELISA assay. Briefly, antibody and mFc tagged human LAG3 ECD recombinant protein were co-incubated with ELISA microplate coated human LSECtin ECD recombinant protein. After washing, HRP labeled anti-mouse Fc 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Blocking ratio was determined by blockade of human LAG3 ECD recombinant protein binding to ELISA microplate coated human LSECtin ECD recombinant protein. As shown in FIG. 3, 021c, 023c, 029c and BMS-986016 can potently block LAG3 and LSECtin interaction.

### 4.2.4. LAG3/FGL1 Interaction Blocking Activity Detection

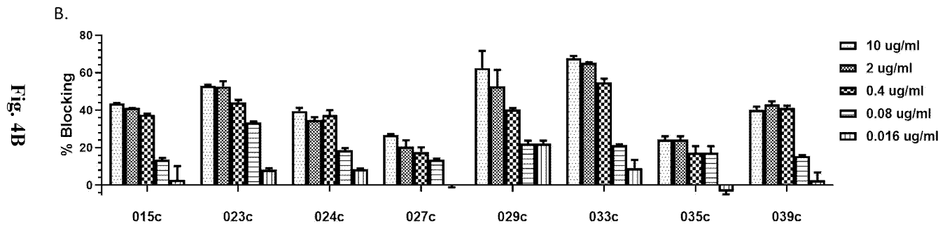

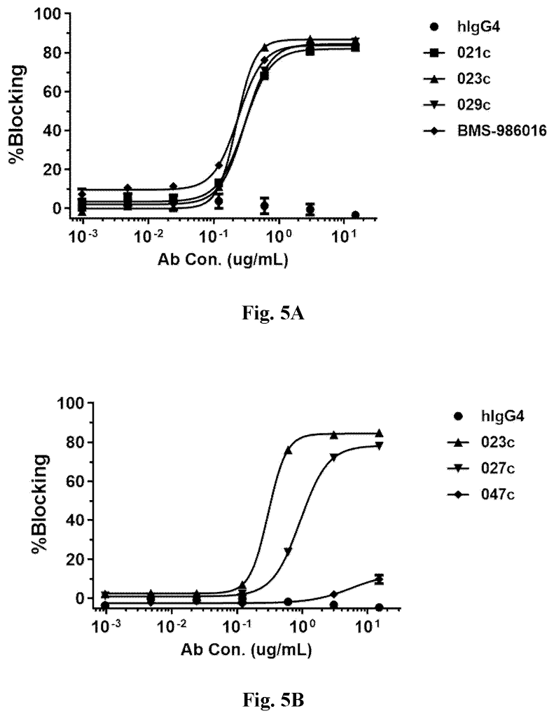

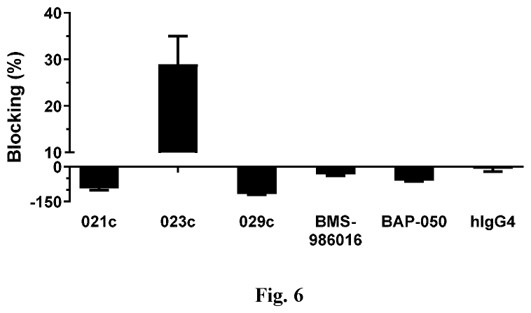

Activity of the purified chimeric antibodies to block LAG3 and FGL1 interaction was determined by competition assays using 3 different formats. In format I, antibody and His tagged human FGL1 recombinant protein were co-incubated with ELISA microplate coated human LAG3 ECD recombinant protein. After washing, HRP labeled anti-His 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Blocking ratio was determined by blockade of human FGL1 recombinant protein binding to ELISA microplate coated human LAG3 ECD recombinant protein. As shown in FIGS. 4A and 4B and Table 11, other than 047c, all the other antibodies as tested can block LAG3 and FGL1 interaction at different levels under format I condition. In format II, antibody and biotin labeled hFc tagged LAG3 ECD recombinant protein were co-incubated with ELISA microplate coated His tagged human FGL1 recombinant protein. After washing, HRP labeled anti-SA 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Blocking ratio was determined by blockade of human LAG3 ECD recombinant protein binding to ELISA microplate coated human FGL1 recombinant protein. IC₅₀ and top blocking ratio calculated using GraphPad Prism9.0 are summarized in Table 11. Under format II condition, 021c, 023c, 029c and BMS-986016 can block LAG3 and FGL1 interaction with comparable potency (FIG. 5A), while 027c showed weaker activity and 047c hardly worked (FIG. 5B). The ranking is consistent with the one got under format I condition. The competition assay of format III is a competitive FACS assay. Briefly, antibody and His tagged human FGL1 recombinant protein were co-incubated with 293F cells stably expressing human LAG3. After washing, dye labeled anti-His 2^{nd} antibody (Sigma) was added and incubated at 37° C. for 1 hour. Fluorescence intensity was detected. Blocking ratio was determined by blockade of human FGL1 recombinant protein binding to LAG3 expressed 293F cells. As shown in FIG. 6 and Table 11, only 023c worked under format III condition, indicating it may have better potency to block LAG3 and FGL1 interaction.

TABLE 11
LAG3/FGL1 blocking activity summary
of anti-LAG3 chimeric antibodies

| | LAG3/FGL1 interaction blocking | | | |
|---|---|---|---|---|
| | Format I | Format II | | Format III |
| | Blocking ratio | IC50 | TOP | Blocking ratio at |
| Ab Code | at 66.7 nM (%) | (nM) | blocking (%) | 200 nM (%) |
| 015c | 43.6 | N/A | N/A | N/A |
| 021c | N/A | 1.93 | 82.0 | no blocking |
| 023c | 52.9 | 1.47 | 86.8 | 29.0 |
| 024c | 39.4 | N/A | N/A | N/A |
| 027c | 26.7 | 6.13 | 78.3 | N/A |
| 029c | 62.6 | 2.00 | 84.5 | no blocking |
| 033c | 67.8 | N/A | N/A | N/A |
| 035c | 24.3 | N/A | N/A | N/A |
| 039c | 40.2 | N/A | N/A | N/A |
| 047c | 14.0 | 35.93 | 12.64 | N/A |
| 048c | 41.2 | N/A | N/A | N/A |
| 057c | 33.3 | N/A | N/A | N/A |
| BMS- | 50.5 | 1.60 | 83.9 | no blocking |
| 986016 | | | | |
| BAP-050 | 39.3 | N/A | N/A | no blocking |

N/A stands for no available data

### 4.2.5. NFAT Reporter Assay

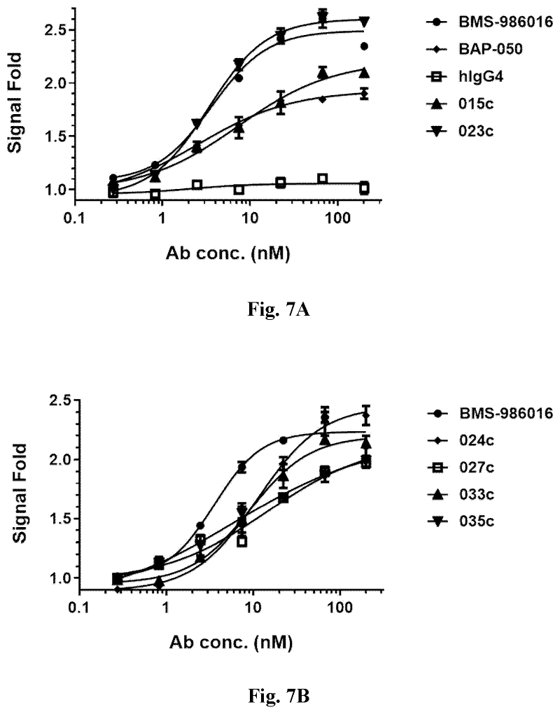

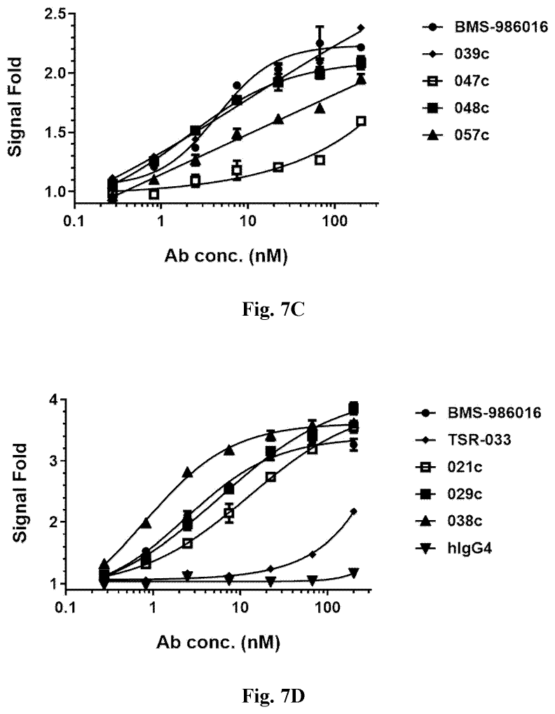

Blocking activity of the purified chimeric antibodies to relieve LAG3/MHC II mediated suppression was assessed by cell based NFAT reporter assay (refer to methods described in Example 3.6). As shown in FIGS. 7A, 7B, 7C and 7D, all antibodies as tested can block LAG3/MIHC II mediated inhibition of reporter gene activity at different levels.

### 4.2.6. Antigen Specific T Cell Activation Assay

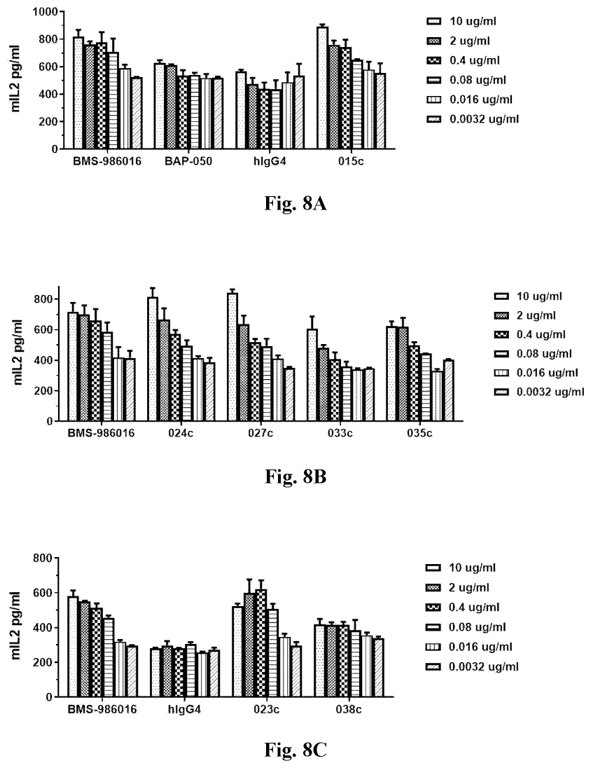

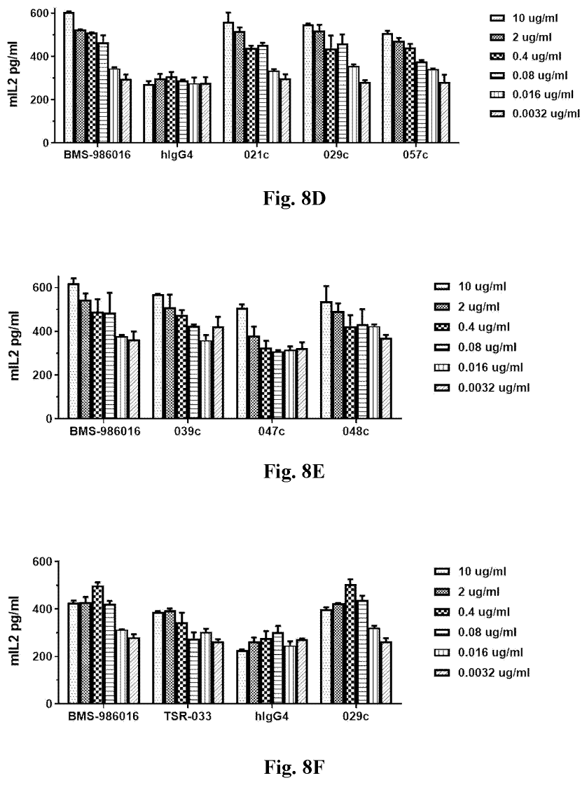

Functional activity of the purified chimeric antibodies to relieve LAG3/MHC II mediated suppression was assessed by antigen specific T cell activation assay (refer to methods described in Example 3.7). As shown in FIGS. 8A, 8B, 8C, 8D, 8E and 8F, all antibodies as tested can block LAG3/MHC II inhibited antigen-pulsed IL-2 secretion of T cells at different levels.

### 4.2.7. Epitope Analysis

Competitive ELISA assay was used for epitope binning of purified chimeric antibodies (Table 12, refer to methods described in Example 3.8). BMS-986016 can't compete BAP-050 to bind to human LAG3, indicating they bind to different epitopes. 023c can compete BMS-986016 but not BAP-050 each other to bind to human LAG3, indicating 023c may have related binding epitopes as BMS-986016. 021c can compete BAP-050 but not BMS-986016 each other to bind to human LAG3, indicating it may have related binding epitopes as BAP-50. 029c can compete both BAP-050 and BMS-986016 (partially) to bind to human LAG3, which is not fully consistent with the result got using mouse hybridoma antibody 029 (Table 9). Although 023c, 029c and 021c have different competition profile against BMS-986016 and BAP-050, they can compete each other to bind to human LAG3, indicating their binding epitopes are not identical but closely related.

TABLE 12
Epitope binning summary of anti-LAG3 chimeric antibodies
% competition

| | Competitor | | | | |
|---|---|---|---|---|---|
| Coating | BMS-986016 | 023c | 029c | 021c | BAP-050 |
| BMS-986016 | 81.39 | 62.89 | 39.86 | 15.66 | 5.56 |
| 023c | 60.18 | 88.11 | 81.84 | 95.36 | 18.87 |
| 029c | 18.93 | 82.56 | 77.79 | 79.41 | 50.71 |
| 021c | −3.43 | 81.59 | 70.78 | 78.02 | 75.78 |
| BAP-050 | −6.49 | 5.95 | 68.44 | 87.69 | 80.74 |

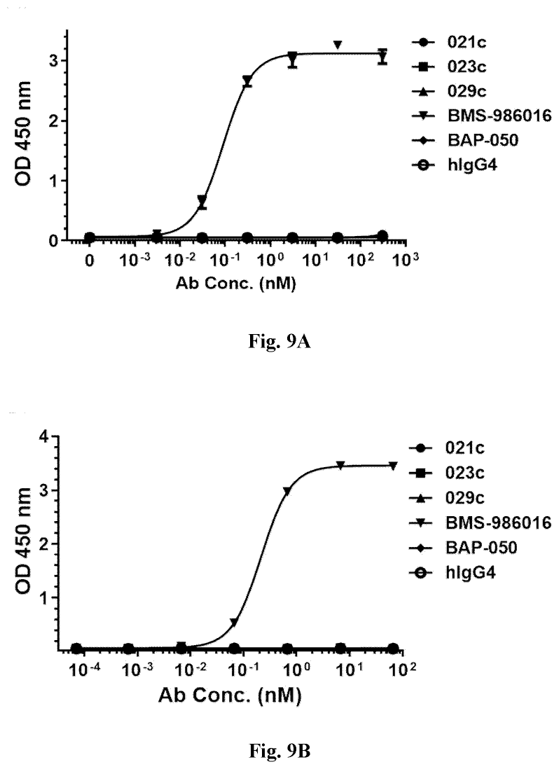

According to patent WO2010/019570A2, BMS-986016 binds to LAG3 D1 domain extra loop having the amino acid sequence GPPAAAPGHPLAPGPHPAAPSSWGPRPRRY. To examine the binding of 021c, 023c and 029c to this region of LAG3, ELISA binding experiments using 2 different formats were performed. In ELISA binding assay of format I (FIG. 9A), biotin labelled LAG3 D1 domain extra loop peptide was incubated with ELISA microplate coated anti-LAG3 antibodies. After washing, HRP labeled anti-SA 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. In ELISA binding assay of format II (FIG. 9B), biotin labelled LAG3 D1 domain extra loop peptide and anti-LAG3 antibodies were co-incubated in ELISA microplate coated with SA. After washing, HRP labeled anti-human Fc 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. As shown in FIGS. 9A and 9B, only BMS-986016 binds to LAG3 D1 domain extra loop, indicating all the other antibodies as tested have different binding epitopes from BMS-986016, although 023c compete BMS-986016 in competitive ELISA assay.

### 4.2.8. Affinity Detection

The purified chimeric antibodies were characterized for binding affinity against human LAG3 using Bio-Layer Interferometry technology (Octet system). The association and dissociation curves were fit with 1:1 binding model, and the Ka/Kd/KD values for each antibody were calculated. The affinity data of Ka/Kd/KD values for each antibody are summarized in Table 13.

TABLE 13
anti-LAG3 chimeric antibodies affinity summary

| Antibody | ka (1/Ms) | kd (1/s) | KD (M) |
|---|---|---|---|
| 021c | 1.39E+05 | 9.05E−04 | 6.52E−09 |
| 023c | 3.59E+05 | 2.33E−04 | 6.48E−10 |
| 029c | 3.89E+05 | 1.39E−03 | 3.58E−09 |
| BMS-986016 | 2.11E+05 | 3.38E−04 | 1.61E−09 |
| TSR-033 | 2.48E+05 | 6.74E−04 | 2.72E−09 |

### 4.2.9. In Vivo Anti-Tumor Activity

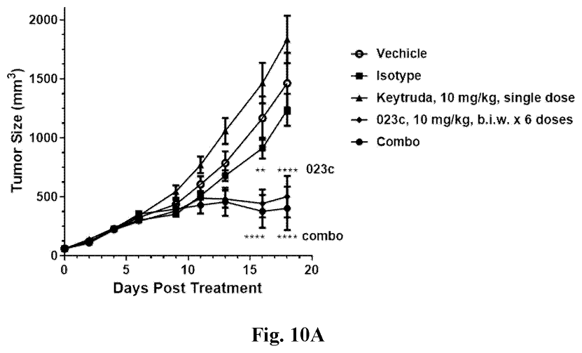

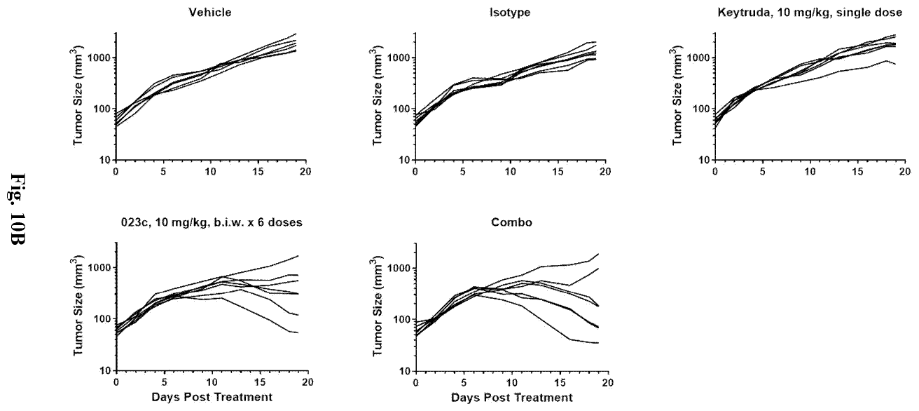

Human PD-1/human LAG3 double knock-in BALB/c mice were inoculated with EMT-6 cells. Treatment groups include vehicle (PBS), isotype control, 10 mg per kg (mpk) Keytruda (pembrolizumab), 10 mpk 023c and combination of 10 mpk Keytruda plus 10 mpk 023c. Treatment was initiated when tumors reached an average volume of 70-75 mm³. Keytruda was administrated intraperitoneally (IP) only once when treatment initiated. 023c was administrated IP twice a week for 6 times. Tumor volume was measured three times per week. Statistics were carried out by two-way anova comparing the mean tumor volume of different treatment groups to that of isotype control group. Average tumor growth curves of different treatment groups (FIG. 10A) show both 10 mpk 023c monotherapy and combination of 10 Keytruda plus 10 mpk 023c significantly inhibited EMT-6 tumor growth. The individual tumor growth curves (FIG. 10B) show 4 of 7 tumors in 023c treatment group and 5 of 7 tumors in combination treatment group shrank.

### Example 5. Antibody Humanization

### 5.1. Humanization of 023c

### 5.1.1 Humanization Design

CDR grafting method was used for humanization of 023c. Briefly, IGHV1-46*01, IGH1V7-4-1*02 and IGKV3-11*01 were first selected as humanization templates for heavy and light chain respectively, based on their homology to the original mouse antibody sequences. CDRs were then defined using Kabat definition. For grafting, potential hotspots removed CDRs and different combinations of canonical residues from 023c were grafted onto the templates and 15 variants (human IgG4 with S228P mutation) were expressed. The humanized antibodies are designated as hu023.01 to hu023.15, where the prefix “hu” indicates “humanized”, and the number in the suffix denotes the serial number of the humanized antibody. All the variants produced were characterized for binding affinity against human LAG3 using Bio-Layer Interferometry technology (Octet system). As shown in Table 14, the humanized antibodies strongly binding to human LAG3 were advanced into further characterization.

TABLE 14
Affinity summary of 023c originated humanized antibodies

| | Octet | | | Biacore | | |
|---|---|---|---|---|---|---|
| Antibody | ka (1/Ms) | kd (1/s) | KD (M) | ka (1/Ms) | kd (1/s) | KD (M) |
| 023c | 3.52E+05 | 1.61E−04 | 4.56E−10 | 5.68E+06 | 4.41E−04 | 7.75E−11 |
| hu023.04 | 3.05E+05 | 1.64E−04 | 5.37E−10 | 1.66E+06 | 4.02E−04 | 2.42E−10 |
| hu023.05 | 3.21E+05 | 1.98E−04 | 6.16E−10 | N/A | N/A | N/A |
| hu023.06 | 2.46E+05 | 1.61E−04 | 6.57E−10 | N/A | N/A | N/A |
| hu023.07 | 2.59E+05 | 1.53E−04 | 5.91E−10 | N/A | N/A | N/A |
| hu023.08 | 3.18E+05 | 2.19E−04 | 6.89E−10 | N/A | N/A | N/A |
| hu023.09 | 2.90E+05 | 2.04E−04 | 7.04E−10 | N/A | N/A | N/A |
| hu023.10 | 2.84E+05 | 7.96E−05 | 2.81E−10 | N/A | N/A | N/A |
| hu023.11 | 3.03E+05 | 2.37E−05 | 7.82E−11 | 4.43E+06 | 9.10E−05 | 2.05E−11 |
| hu023.12 | 2.96E+05 | 5.58E−05 | 1.89E−10 | N/A | N/A | N/A |
| hu023.13 | 3.03E+05 | 3.67E−05 | 1.21E−10 | N/A | N/A | N/A |
| hu023.14 | 3.22E+05 | 7.72E−05 | 2.40E−10 | N/A | N/A | N/A |

N/A stands for no available data

5.1.2 Characterization of Humanized Antibodies Originated from 023c

### 5.1.2.1. Binding Activity Detection

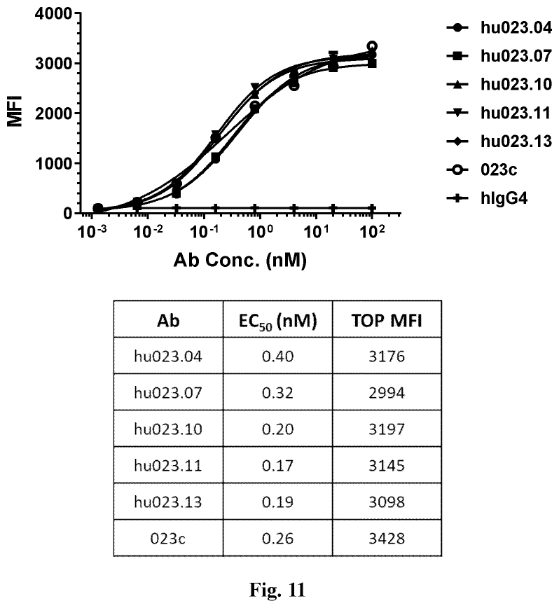

Binding activity of the humanized antibodies originated from 023c was determined by FACS assay using 293F cells stably expressing human LAG3 (refer to Example 3.3). MFI was detected and EC₅₀ was calculated using GraphPad Prism9.0. As shown in FIG. 11, all the humanized antibodies as tested were confirmed to retain the similar activity as the parental antibody 023c to bind to human LAG3.

### 5.1.2.2. LAG3/MHC II Interaction Blocking Activity Detection

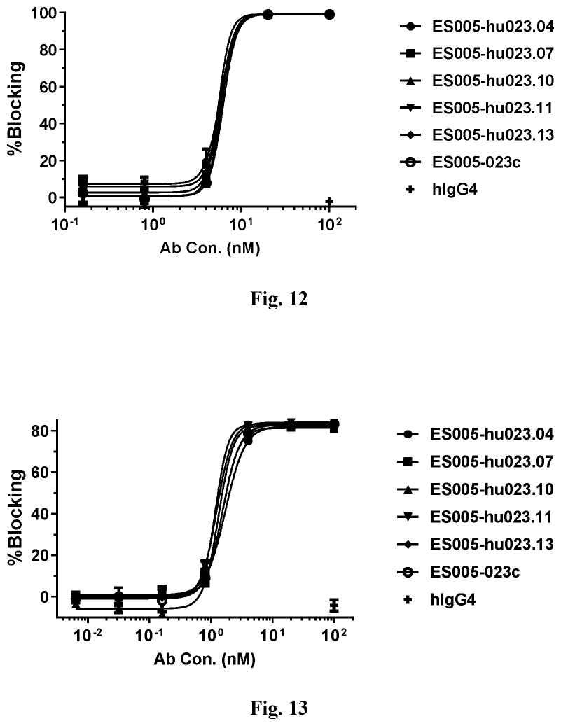

Activity of 023c originated humanized antibodies to block LAG3 and MHC II interaction was determined by competitive FACS assay (FIG. 12, refer to Example 4.2.2). IC₅₀ and top blocking ratio calculated using GraphPad Prism9.0 are summarized in Table 15. All the humanized antibodies as tested were confirmed to retain the similar activity as the parental antibody 023c to block LAG3 and MHC II interaction.

TABLE 15
LAG3/MHC II and LAG3/FGL1 interaction blocking activity
of 023c originated humanized antibodies

| | LAG3/MHC II interaction | | LAG3/FGL1 interaction | |
|---|---|---|---|---|
| | blocking | | blocking (Format II) | |
| | | TOP blocking | | TOP blocking |
| Ab Code | IC₅₀ (nM) | (%) | IC₅₀ (nM) | (%) |
| hu023.04 | 5.9 | 99.2 | 1.7 | 82.2 |
| hu023.07 | 5.5 | 99.2 | 1.5 | 81.3 |
| hu023.10 | 5.9 | 99.2 | 1.3 | 82.8 |
| hu023.11 | 5.8 | 99.3 | 1.2 | 83.9 |
| hu023.13 | 5.7 | 99.3 | 1.2 | 83.2 |
| 023c | 6.2 | 99.2 | 1.5 | 82.8 |

### 5.1.2.3. LAG3/FGL1 Interaction Blocking Activity Detection

Activity of 023c originated humanized antibodies to block LAG3 and FGL1 interaction was determined by competitive ELISA assay of format II (FIG. 13, refer to format condition in Example 4.2.4). IC₅₀ and top blocking ratio calculated using GraphPad Prism9.0 are summarized in Table 15. All the humanized antibodies as tested were confirmed to retain the similar activity as the parental antibody 023c to block LAG3 and FGL1 interaction.

### 5.1.2.4. NFAT Reporter Assay

Blocking activity of hu023.04 and hu023.11 to relieve LAG3/MHC II mediated suppression was assessed by cell based NFAT reporter assay (FIG. 14, refer to methods described in Example 3.6). IC₅₀ and top activity fold change were calculated using GraphPad Prism9.0. Hu023.11 was confirmed to retain the similar activity to the parental antibody 023c to relieve LAG3/MHC II mediated suppression of NFAT reporter gene activity, while hu023.04 showed slightly weaker potency.

### 5.1.2.5. Antigen Specific T Cell Activation Assay

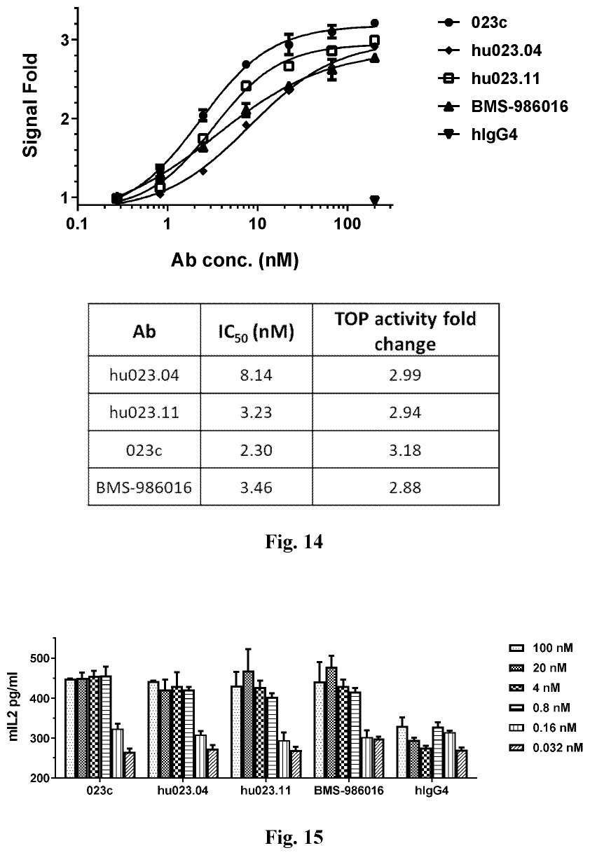

Functional activity of hu023.04 and hu023.11 to relieve LAG3/MHC II mediated suppression was assessed by antigen specific T cell activation assay (refer to methods described in Example 3.7). As shown in FIG. 15, hu023.04 and hu023.11 were confirmed to have similar activity as BMS-986016 and the parental antibody 023c to block LAG3/MHC II inhibited antigen-pulsed IL-2 secretion of T cells.

### 5.1.2.6. Affinity Detection

Hu023.04 and hu023.11 were further characterized for binding affinity against human LAG3 using Surface Plasmon Resonance technology (Biacore system). The association and dissociation curves were fit with 1:1 binding model, and the Ka/Kd/KD values for each antibody were calculated. As summarized in Table 14, hu023.04 was confirmed to retain similar human LAG3 binding affinity to the parental antibody 023c, while hu023.11 showed slightly higher affinity than ES005-023c.

### 5.1.2.7. Epitope Analysis

The binding epitope of hu023.11 on LAG3 was further mapped using hydrogen deuterium exchange mass spectrometry (HDX-MS). The binding of hu023.11 resulted in reduced hydrogen deuterium exchange in the peptide “AAVHLRDRALSCRL”, indicating that this region on LAG3 may be critical for hu023.11 to bind.

### 5.2. Humanization of 021c

### 5.2.1 Humanization Design

CDR grafting method was used for humanization of 021c. Briefly, IGHV4-31*01 was first selected as humanization template for heavy chain while IGKV3-11*01 and IGKV4-1*01 were selected as humanization templates for light chain, based on their homology to the original mouse sequences. CDRs were then defined using Kabat definition. For grafting, two methods were used to identify variants with the best combinations of canonical residues to retain the functional properties of the original antibody after humanization. In the first method, potential hotspots removed CDR sequences and different computationally designed combinations of canonical residues from 021c were grafted onto the templates and expressed in a 96-well high-throughput protein expression system. All the variants produced were then tested in multiple in vitro assays to select the best ones that retained the functional properties of 021c. In the second method, in addition to direct grafting of potential hotspots removed CDR sequences onto templates, all canonical positions of the grafted sequences were given either the mouse or the template amino acid during gene synthesis. The resulting libraries, which theoretically contained all possible combinations of canonical amino acids, were displayed on yeast cell surface and the best variants were selected based on their binding to the LAG3 antigen. Variants identified this way were similarly expressed and assayed as in the first method. For certain variants, back mutations obtained from both methods were combined.

5.2.2. Characterization of Humanized Antibodies Originated from 021c

### 5.2.2.1. Binding Activity Detection

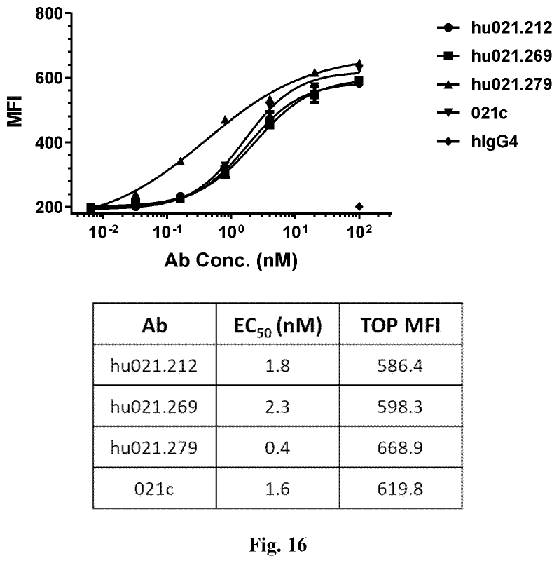

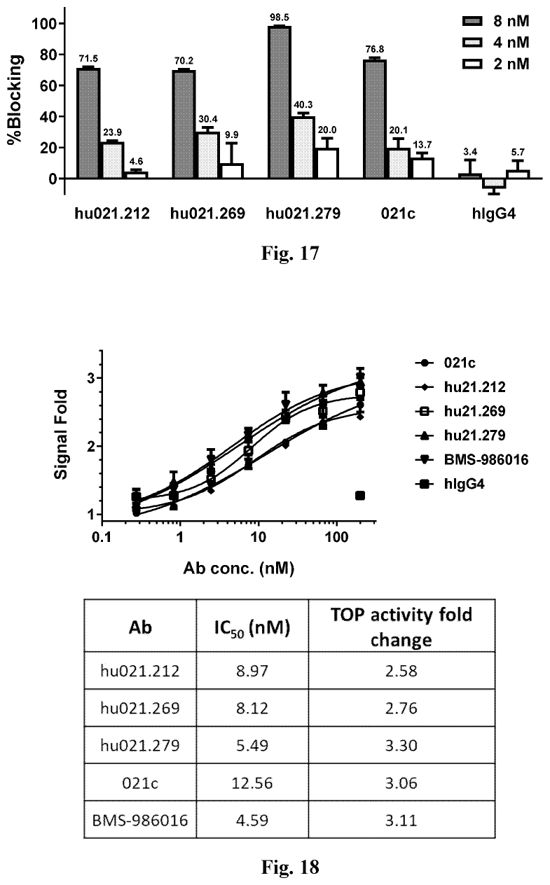

Binding activity of 021c originated humanized antibodies were determined by FACS assay using CHOK1 cells stably expressing human LAG3 (FIG. 16, refer to Example 3.3), where the prefix “hu” indicates “humanized”, and the number in the suffix denotes the serial number of the humanized antibody. MFI was detected and EC₅₀ was calculated using GraphPad Prism9.0. Hu021.212 and hu021.269 were confirmed to retain the similar activity as the parental antibody 021c to bind to human LAG3, while hu021.279 showed better activity than 021c.

### 5.2.2.2. LAG3/MHC II Interaction Blocking Activity Detection

Activity of 021c originated humanized antibodies to block LAG3 and MHC II interaction was determined by competitive FACS assay (refer to Example 4.2.2). As shown in FIG. 17, hu021.212 and hu021.269 were confirmed to retain the similar activity as the parental antibody 021c to block LAG3 and MHC II interaction, while hu021.279 showed better activity than 021c.

### 5.2.2.3. NFAT Reporter Assay

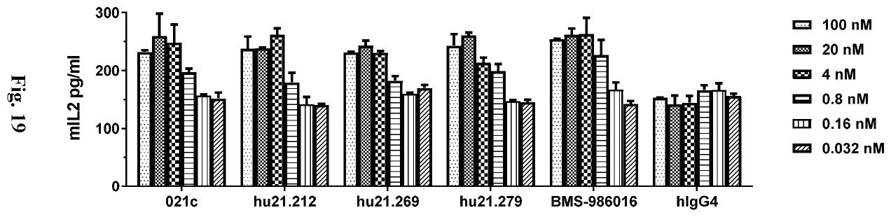

Blocking activity of 021c originated humanized antibodies to relieve LAG3/MHC II mediated suppression was assessed by cell based NFAT reporter assay (refer to methods described in Example 3.6). IC₅₀ and top activity fold change were calculated using GraphPad Prism9.0. As shown in FIG. 18, compared with the parental antibody 021c, hu021.212, hu021.269 and hu021.279 were confirmed to have similar or better activity to block LAG3/MHC II mediated inhibition of reporter gene activity.

### 5.2.2.4. Antigen Specific T Cell Activation Assay

Functional activity of 021c originated humanized antibodies to relieve LAG3/MHC II mediated suppression was assessed by antigen specific T cell activation assay (refer to methods described in Example 3.7). As shown in FIG. 19, hu021.212, hu021.269 and hu021.279 were confirmed to have similar activity as BMS-986016 and the parental antibody 021c to block LAG3/MHC II inhibited antigen-pulsed IL-2 secretion of T cells.

### 5.2.2.5. Affinity Detection

Hu021.269 and hu021.279 were characterized for binding affinity against human LAG3 using Bio-Layer Interferometry technology (Octet system). The association and dissociation curves were fit with 1:1 binding model, and the Ka/Kd/KD values for each antibody were calculated. As summarized in Table 16, hu021.269 and hu021.279 were confirmed to retain the similar affinity as parental antibody 021c to bind to human LAG3.

TABLE 16
Affinity summary of 021c and 029c
originated humanized antibodies

| | Octet | | |
|---|---|---|---|
| Antibody | ka (1/Ms) | kd (1/s) | KD (M) |
| hu021.269 | 4.51E+05 | 3.14E−03 | 6.95E−09 |
| hu021.279 | 8.77E+05 | 3.13E−03 | 3.57E−09 |
| 021c | 5.08E+05 | 2.71E−03 | 5.34E−09 |
| hu029.40 | 4.68E+06 | 2.24E−02 | 4.79E−09 |
| hu029.53 | 4.38E+06 | 2.17E−02 | 4.94E−09 |
| hu029.55 | 4.47E+06 | 1.97E−02 | 4.42E−09 |
| hu029.55.S93Q | 5.06E+06 | 2.07E−02 | 4.08E−09 |
| 029c | 3.48E+06 | 2.68E−02 | 7.7E−09 |

### 5.3. Humanization of 029c

### 5.3.1. Humanization Design

CDR grafting method was used for humanization of 029c. Briefly, IGHV3-11*01 was first selected as humanization template for heavy chain while IGKV1-12*01 were first selected as humanization template for light chain, based on their homology to the original mouse sequences. CDRs were then defined using Kabat definition. For grafting, two methods were used to identify variants with the best combinations of canonical residues to retain the functional properties of the original antibody after humanization. In the first method, CDR sequences and different computationally designed combinations of canonical residues from 029c were grafted onto the templates and expressed in a 96-well high-throughput protein expression system. All the variants produced were then tested in multiple in vitro assays to select the best ones that retained the functional properties of 029c. In the second method, in addition to direct grafting of CDR sequences onto templates, all canonical positions of the grafted sequences were given either the mouse or the template amino acid during gene synthesis. The resulting libraries, which theoretically contained all possible combinations of canonical amino acids, were displayed on yeast cell surface and the best variants were selected based on their binding to the LAG3 antigen. Variants identified this way were similarly expressed and assayed as in the first method. For certain variants, back mutations obtained from both methods were combined.

5.3.2. Characterization of Humanized Antibodies Originated from 029c

### 5.3.2.1. Binding Activity Detection

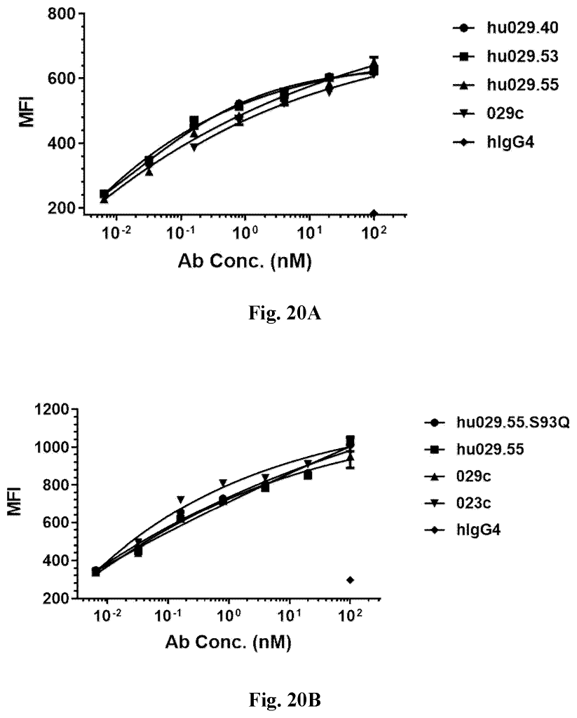

Binding activity of 029c originated humanized antibodies were determined by FACS assay using CHOK1 cells stably expressing human LAG3 (FIG. 20, refer to Example 3.3), where the prefix “hu” indicates “humanized”, the number in the suffix denotes the serial number of the humanized antibody, and the suffix S93Q denotes the replacement of Serine at position 93 with Glutamine to remove potential hotspot. As shown in FIGS. 20A and 20B, hu029.40, hu029.53, hu029.55 and hu029.55.S93Q were confirmed to retain the similar activity as the parental antibody 029c to bind to human LAG3.

### 5.3.2.2. LAG3/MHC II Interaction Blocking Activity Detection

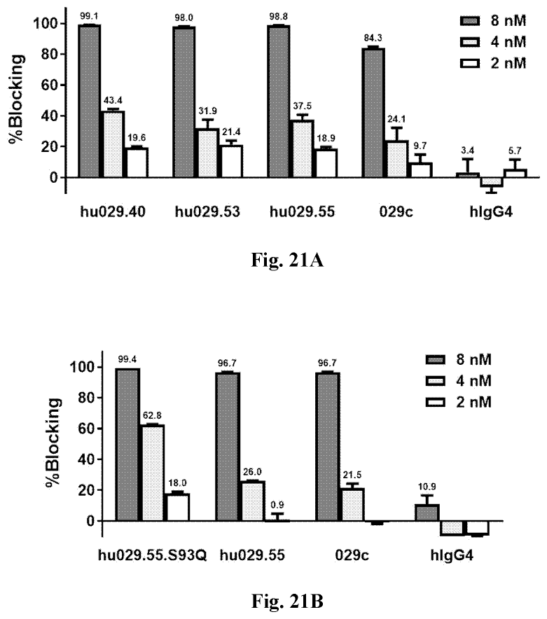

Activity of 029c originated humanized antibodies to block LAG3 and MHC II interaction was determined by competitive FACS assay (refer to Example 4.2.2). As shown in FIGS. 21A and 21B, hu029.40, hu029.53, hu029.55 and hu029.55.S93Q were confirmed to retain the similar activity as the parental antibody 029c to block LAG3 and MHC II interaction.

### 5.3.2.3. NFAT Reporter Assay

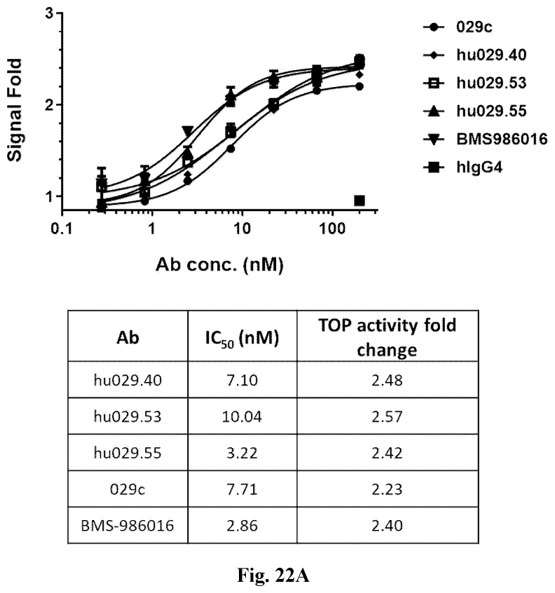

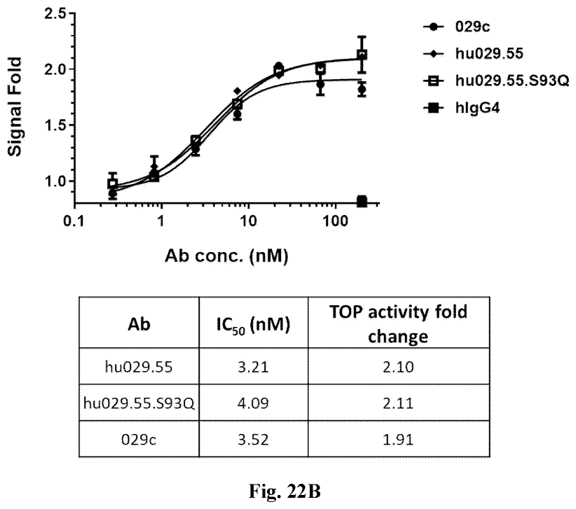

Blocking activity of 029c originated humanized antibodies to relieve LAG3/MHC II mediated suppression was assessed by cell based NFAT reporter assay (refer to methods described in Example 3.6). IC₅₀ and top activity fold change were calculated using GraphPad Prism9.0. As shown in FIGS. 22A and 22B, compared with the parental antibody 029c, hu029.40, hu029.53, hu029.55 and hu029.55.S93Q were confirmed to have similar or better activity to block LAG3/MHC II mediated inhibition of reporter gene activity.

### 5.3.2.4. Antigen Specific T Cell Activation Assay

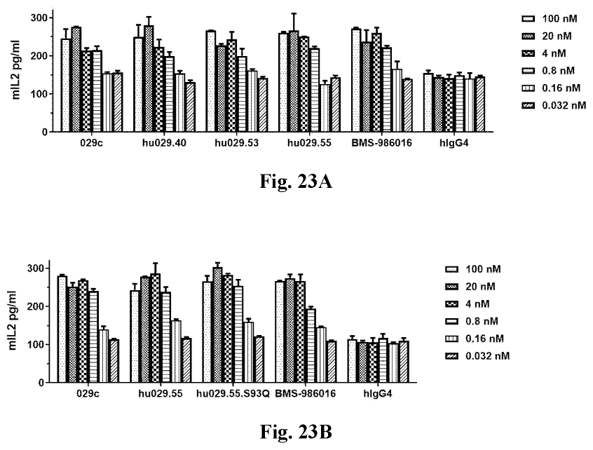

Functional activity of 029c originated humanized antibodies to relieve LAG3/MHC II mediated suppression was assessed by antigen specific T cell activation assay (refer to methods described in Example 3.7). As shown in FIGS. 23A and 23B, hu029.40, hu029.53, hu029.55 and hu029.55.S93Q were confirmed to have similar activity as BMS-986016 and the parental antibody 029c to block LAG3/MHC II inhibited antigen-pulsed IL-2 secretion of T cells.

### 5.3.2.5. Affinity Detection

029c originated humanized antibodies were characterized for binding affinity against human LAG3 using Bio-Layer Interferometry technology (Octet system). The association and dissociation curves were fit with 1:1 binding model, and the Ka/Kd/KD values for each antibody were calculated. As summarized in Table 16, 029c originated humanized antibodies were confirmed to retain the similar affinity as parental antibody 029c to bind to human LAG3.

### 5.4. In Vivo Efficacy Evaluation

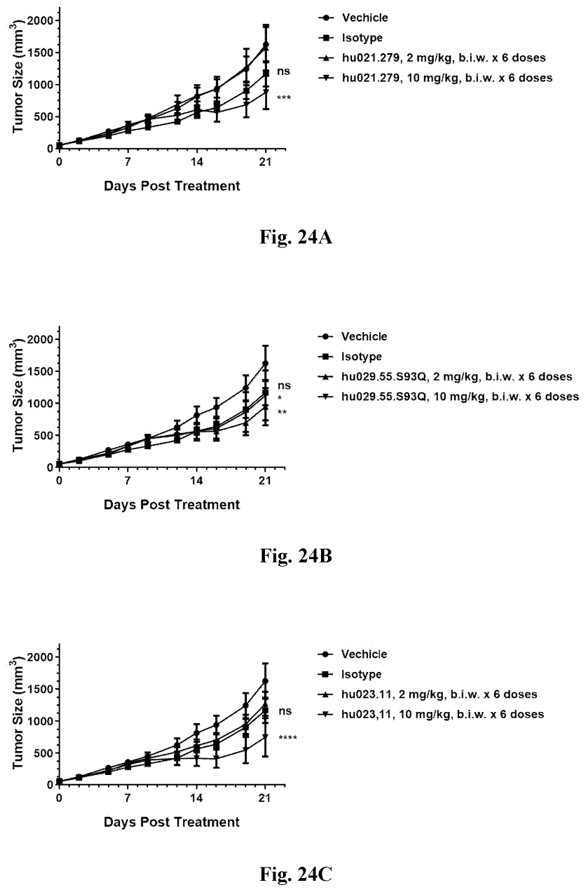

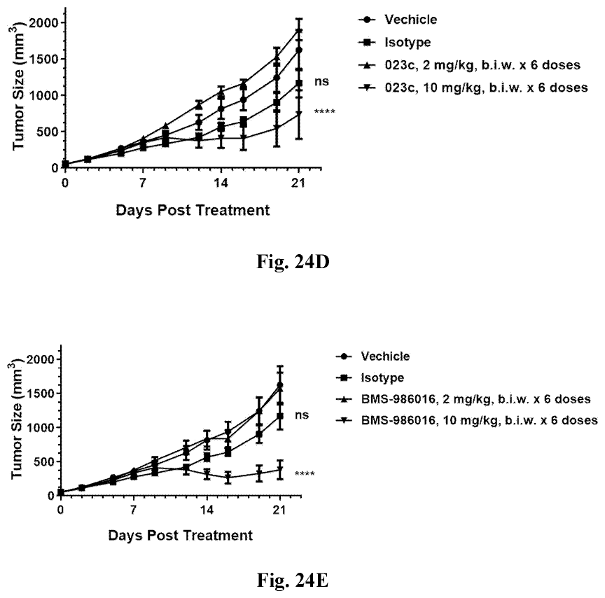

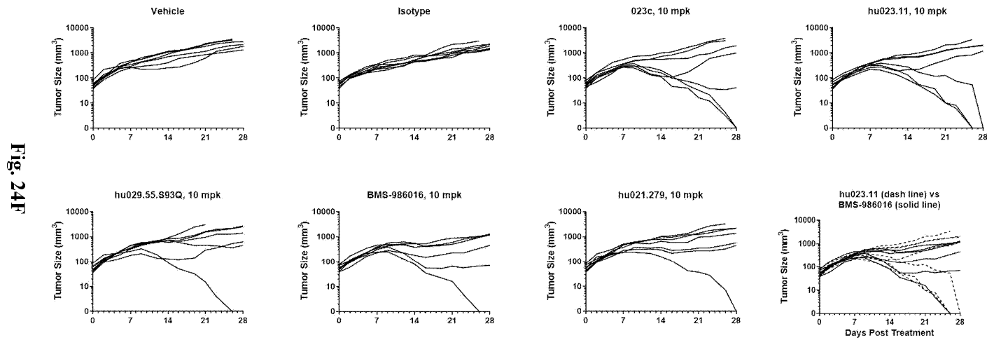

Human PD-1/human LAG3 double knock-in BALB/c mice were inoculated with EMT-6 cells. Treatment groups include vehicle (PBS), isotype control, 2 or 10 mpk hu21.279, hu029.55.S93Q, hu023.11, 023c, and BMS-986016. Treatment was initiated when tumors reached an average volume of 70-75 mm³. Anti-LAG3 antibodies were administrated IP twice a week for 6 times. Tumor volume was measured three times per week. Statistics were carried out by two-way anova comparing the mean tumor volume of different treatment groups to that of vehicle group. 10 mpk of hu021.279 (FIG. 24A), hu029.55.S93Q (FIG. 24B), hu023.11 (FIG. 24C), 023c (FIG. 24D) and BMS-986016 inhibited EMT-6 tumor growth in vivo at different levels. Hu023.11 showed similar anti-tumor effect as 023c. 1 of 7 mice in 10 mpk hu021.279 treatment group, 1 of 7 mice in 10 mpk hu029.55.S93Q treatment group, 1 of 7 mice in 10 mpk BMS-986016 treatment group, 2 of 7 mice in 10 mpk 023c treatment group and 3 of 7 mice in hu023.11 treatment group were tumor free at the end of the study (FIG. 24F).

1. An anti-LAG3 antibody or antigen-binding fragment thereof, comprising heavy chain HCDR1, HCDR2 and HCDR3, and/or light chain LCDR1, LCDR2 and LCDR3, wherein
(a) the HCDR1 comprises an amino acid sequence of SGYYWX₁₆ (SEQ ID NO: 234),
the HCDR2 comprises an amino acid sequence of
(SEQ ID NO: 235)
DISYX₁₇X₁₈GNNYNPSLKN,
the HCDR3 comprises an amino acid sequence of SEQ ID NO: 11,
the LCDR1 comprises an amino acid sequence of SEQ ID NO: 12,
the LCDR2 comprises an amino acid sequence of RASNX₁₉EX₂₀ (SEQ ID NO: 236), and
the LCDR3 comprises an amino acid sequence of SEQ ID NO: 14,
wherein, X₁₆ is N or T, X₁₇ is E or D, X₁₈ is G or A, X₁₉ is L or R, X₂₀ is T or S;
(b) the HCDR1 comprises an amino acid sequence of SEQ ID NO: 40,
the HCDR2 comprises an amino acid sequence of SEQ ID NO: 41,
the HCDR3 comprises an amino acid sequence of SEQ ID NO: 42,
the LCDR1 comprises an amino acid sequence of RSSX₁₄NIVHX₁₅DGNTYLE SEQ ID NO: 43,
the LCDR2 comprises an amino acid sequence of SEQ ID NO: 21, and
the LCDR3 comprises an amino acid sequence of QQHDX₂₁SPWT (SEQ ID NO: 237),
wherein, X₂₁ is S or Q;
(c) the HCDR1 comprises an amino acid sequence of SEQ ID NO: 17,
the HCDR2 comprises an amino acid sequence of
(SEQ ID NO: 238)
X₂₂IYTDTGEPTYAEEFKG,
the HCDR3 comprises an amino acid sequence of SEQ ID NO: 19,
the LCDR1 comprises an amino acid sequence of SEQ ID NO: 20,
the LCDR2 comprises an amino acid sequence of SEQ ID NO: 21, and
the LCDR3 comprises an amino acid sequence of QQHYNX₂₃PPT (SEQ ID NO: 239),
wherein, X₂₂ is M or I, X₂₃ is A or S;
(d) the HCDR1 comprises an amino acid sequence of SYGX₁N (SEQ ID NO: 226),
the HCDR2 comprises an amino acid sequence of
(SEQ ID NO: 227)
EIYPRSGNTYYNEX₂X₃X₄X₅,
the HCDR3 comprises an amino acid sequence of
(SEQ ID NO: 228)
GGTYDGYYYAMDX₆,
the LCDR1 comprises an amino acid sequence of
(SEQ ID NO: 229)
RASESVDNFGSSFX₇H,
the LCDR2 comprises an amino acid sequence of SEQ ID NO: 13, and
the LCDR3 comprises an amino acid sequence of SEQ ID NO: 58,
wherein, X₁ is I or V, X is K or R X₃ is F or L, X₄ is K or G, X is D or G, X₆ is F or Y, X₇ is L or M;
(e) the HCDR1 comprises an amino acid sequence of DYNX₈N (SEQ ID NO: 230),
the HCDR2 comprises an amino acid sequence of
(SEQ ID NO: 231)
LVDPIYGTIRYNQX₉FKX10
the HCDR3 comprises an amino acid sequence of
(SEQ ID NO: 232)
IX₁₁TX₁₂VRYFDX₁₃,
the LCDR1 comprises an amino acid sequence of
(SEQ ID NO: 233)
RSSX₁₄NIVHX₁₅DGNTYLE,
the LCDR2 comprises an amino acid sequence of SEQ ID NO: 36, and
the LCDR3 comprises an amino acid sequence of SEQ ID NO: 72,
wherein, X₈ is L or M, X₉ is N or K, X₁₀ is D or G, X₁₁ is M or T, X₁₂ is A or S, X₁₃ is H or Y, X₁₄ is L or Q, X₁₅ is S or T;
(f) the HCDR1 comprises an amino acid sequence of SEQ ID NO: 1,
the HCDR2 comprises an amino acid sequence of SEQ ID NO: 2,
the HCDR3 comprises an amino acid sequence of SEQ ID NO: 3,
the LCDR1 comprises an amino acid sequence of SEQ ID NO: 4,
the LCDR2 comprises an amino acid sequence of SEQ ID NO: 5, and
the LCDR3 comprises an amino acid sequence of SEQ ID NO: 6,
(g) the HCDR1 comprises an amino acid sequence of SEQ ID NO: 1,
the HCDR2 comprises an amino acid sequence of SEQ ID NO: 25,
the HCDR3 comprises an amino acid sequence of SEQ ID NO: 26,
the LCDR1 comprises an amino acid sequence of SEQ ID NO: 27,
the LCDR2 comprises an amino acid sequence of SEQ ID NO: 28, and
the LCDR3 comprises an amino acid sequence of SEQ ID NO: 29,
(h) the HCDR1 comprises an amino acid sequence of SEQ ID NO: 32,
the HCDR2 comprises an amino acid sequence of SEQ ID NO: 33,
the HCDR3 comprises an amino acid sequence of SEQ ID NO: 34,
the LCDR1 comprises an amino acid sequence of SEQ ID NO: 35,
the LCDR2 comprises an amino acid sequence of SEQ ID NO: 36, and
the LCDR3 comprises an amino acid sequence of SEQ ID NO: 37,
(i) the HCDR1 comprises an amino acid sequence of SEQ ID NO: 47,
the HCDR2 comprises an amino acid sequence of SEQ ID NO: 48,
the HCDR3 comprises an amino acid sequence of SEQ ID NO: 49,
the LCDR1 comprises an amino acid sequence of SEQ ID NO: 50,
the LCDR2 comprises an amino acid sequence of SEQ ID NO: 36, and
the LCDR3 comprises an amino acid sequence of SEQ ID NO: 51, or
(j) the HCDR1 comprises an amino acid sequence of SEQ ID NO: 9,
the HCDR2 comprises an amino acid sequence of SEQ ID NO: 61,
the HCDR3 comprises an amino acid sequence of SEQ ID NO: 62,
the LCDR1 comprises an amino acid sequence of SEQ ID NO: 63,
the LCDR2 comprises an amino acid sequence of SEQ ID NO: 64, and
the LCDR3 comprises an amino acid sequence of SEQ ID NO: 65.

2-6. (canceled)

7. The antibody or antigen-binding fragment thereof of claim 1, wherein
(a) the HCDR1 comprises an amino acid sequence of SEQ ID NO: 9; the HCDR2 comprises an amino acid sequence of SEQ ID NO: 10; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 13; and the LCDR3 comprises an amino acid sequence of SEQ ID NO: 14;
(b) the HCDR1 comprises an amino acid sequence of SEQ ID NO: 9; the HCDR2 comprises an amino acid sequence of SEQ ID NO: 90; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 91; and the LCDR3 comprises an amino acid sequence of SEQ ID NO: 14;
(c) the HCDR1 comprises an amino acid sequence of SEQ ID NO: 9; the HCDR2 comprises an amino acid sequence of SEQ ID NO: 10; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 94; and the LCDR3 comprises an amino acid sequence of SEQ ID NO: 14;
(d) the HCDR1 comprises an amino acid sequence of SEQ ID NO: 97; the HCDR2 comprises an amino acid sequence of SEQ ID NO: 98; the HCDR3 comprises an amino acid sequence of SEQ ID NO: 11; the LCDR1 comprises an amino acid sequence of SEQ ID NO: 12; the LCDR2 comprises an amino acid sequence of SEQ ID NO: 13; and the LCDR3 comprises an amino acid sequence of SEQ ID NO: 14;
(e) the HCDR1 comprises an amino acid sequence of SEQ ID NO: 40; the HCDR2 comprises an amino acid sequence of SEQ ID NO: 41; the HCDR3 comprises an amino acid sequence of SEQ ID NO: 42; the LCDR1 comprises an amino acid sequence of SEQ ID NO: 43; the LCDR2 comprises an amino acid sequence of SEQ ID NO: 21; and the LCDR3 comprises an amino acid sequence of SEQ ID NO: 44;
(f) the HCDR1 comprises an amino acid sequence of SEQ ID NO: 40; the HCDR2 comprises an amino acid sequence of SEQ ID NO: 41; the HCDR3 comprises an amino acid sequence of SEQ ID NO: 42; the LCDR1 comprises an amino acid sequence of SEQ ID NO: 43; the LCDR2 comprises an amino acid sequence of SEQ ID NO: 21; and the LCDR3 comprises an amino acid sequence of SEQ ID NO: 106;
(g) the HCDR1 comprises an amino acid sequence of SEQ ID NO: 17; the HCDR2 comprises an amino acid sequence of SEQ ID NO: 18; the HCDR3 comprises an amino acid sequence of SEQ ID NO: 19; the LCDR1 comprises an amino acid sequence of SEQ ID NO: 20; the LCDR2 comprises an amino acid sequence of SEQ ID NO: 21; and the LCDR3 comprises an amino acid sequence of SEQ ID NO: 22;
(h) the HCDR1 comprises an amino acid sequence of SEQ ID NO: 17; the HCDR2 comprises an amino acid sequence of SEQ ID NO: 18; the HCDR3 comprises an amino acid sequence of SEQ ID NO: 19; the LCDR1 comprises an amino acid sequence of SEQ ID NO: 20; the LCDR2 comprises an amino acid sequence of SEQ ID NO: 21; and the LCDR3 comprises an amino acid sequence of SEQ ID NO: 109;
(i) the HCDR1 comprises an amino acid sequence of SEQ ID NO: 54; the HCDR2 comprises an amino acid sequence of SEQ ID NO: 55; the HCDR3 comprises an amino acid sequence of SEQ ID NO: 56; the LCDR1 comprises an amino acid sequence of SEQ ID NO: 57; the LCDR2 comprises an amino acid sequence of SEQ ID NO: 13; and the LCDR3 comprises an amino acid sequence of SEQ ID NO: 58;
(j) the HCDR1 comprises an amino acid sequence of SEQ ID NO: 75; the HCDR2 comprises an amino acid sequence of SEQ ID NO: 76; the HCDR3 comprises an amino acid sequence of SEQ ID NO: 77; the LCDR1 comprises an amino acid sequence of SEQ ID NO: 78; the LCDR2 comprises an amino acid sequence of SEQ ID NO: 13; and the LCDR3 comprises an amino acid sequence of SEQ ID NO: 58;
(k) the HCDR1 comprises an amino acid sequence of SEQ ID NO: 54; the HCDR2 comprises an amino acid sequence of SEQ ID NO: 87; the HCDR3 comprises an amino acid sequence of SEQ ID NO: 77; the LCDR1 comprises an amino acid sequence of SEQ ID NO: 78; the LCDR2 comprises an amino acid sequence of SEQ ID NO: 13; and the LCDR3 comprises an amino acid sequence of SEQ ID NO: 58;
(l) the HCDR1 comprises an amino acid sequence of SEQ ID NO: 68; the HCDR2 comprises an amino acid sequence of SEQ ID NO: 69; the HCDR3 comprises an amino acid sequence of SEQ ID NO: 70; the LCDR1 comprises an amino acid sequence of SEQ ID NO: 71; the LCDR2 comprises an amino acid sequence of SEQ ID NO: 36; and the LCDR3 comprises an amino acid sequence of SEQ ID NO: 72; or
(m) the HCDR1 comprises an amino acid sequence of SEQ ID NO: 81; the HCDR2 comprises an amino acid sequence of SEQ ID NO: 82; the HCDR3 comprises an amino acid sequence of SEQ ID NO: 83; the LCDR1 comprises an amino acid sequence of SEQ ID NO: 84; the LCDR2 comprises an amino acid sequence of SEQ ID NO: 36; and the LCDR3 comprises an amino acid sequence of SEQ ID NO: 72.

8. (canceled)

9. The antibody or antigen-binding fragment thereof of claim 1, comprising a heavy chain variable region (VH) comprising the sequence of SEQ ID NO: 7, 15, 23, 30, 38, 45, 52, 59, 66, 73, 79, 85 88, 92, 95, 99, 101, 103, 105, 110, 114, 115, or 116, or a homologous sequence thereof having at least 80% sequence identity to SEQ ID NO: 7, 15, 23, 30, 38, 45, 52, 59, 66, 73, 79, 85, 88, 92, 95, 99, 101, 103, 105, 110, 114, 115, or 116.

10. The antibody or antigen-binding fragment thereof of claim 1, comprising a light chain variable region (VL) comprising the sequence of SEQ ID NO: 8, 16, 24, 31, 39, 46, 53, 60, 67, 74, 80, 86, 89, or 93, 96, 100, 102, 104, 107, 111, 112, or 113, a homologous sequence thereof having at least 80% sequence identity to SEQ ID NO: 8, 16, 24, 31, 39, 46, 53, 60, 67, 74, 80, 86, 89, 93, 96, 100, 102, 104, 107, 111, 112, or 113.

11-13. (canceled)

14. The antibody or an antigen-binding fragment thereof of claim 1, further comprising an Fc region, optionally an Fc region of human immunoglobulin (Ig), or optionally an Fc region of human IgG.

15-16. (canceled)

17. The antibody or an antigen-binding fragment thereof of claim 1, which is humanized.

18. The antibody or an antigen-binding fragment thereof of claim 1, which is a monoclonal antibody, a bispecific antibody, a multi-specific antibody, a recombinant antibody, a chimeric antibody, a labeled antibody, a bivalent antibody, an anti-idiotypic antibody or a fusion protein.

19. The antibody or an antigen-binding fragment thereof of claim 1, which is a diabody, a Fab, a Fab′, a F(ab′)2, a Fd, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

20-24. (canceled)

25. The antibody or an antigen-binding fragment thereof of claim 1, which is linked to one or more conjugate moieties.

26. The antibody or an antigen-binding fragment thereof of claim 25, wherein the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety, or other anticancer drugs.

27. An isolated polynucleotide encoding the antibody or an antigen-binding fragment thereof of claim 1.

28. A vector comprising the isolated polynucleotide of claim 27.

29. A host cell comprising the vector of claim 28.

30. A pharmaceutical composition, comprising:
(i) the antibody or an antigen-binding fragment thereof of claim 1, or the polynucleotide encoding the antibody or antigen-binding fragment thereof of claim 1, and
(ii) one or more pharmaceutically acceptable carriers.

31. The pharmaceutical composition of claim 30, further comprising an additional therapeutic agent.

32-34. (canceled)

35. A method of expressing the antibody or an antigen-binding fragment thereof of claim 1, comprising culturing the host cell of claim 29 under the condition at which the vector of claim 28 is expressed.

36. A method of treating, preventing or alleviating a disease associated with a suppressed immune system in a subject, comprising administering to the subject a therapeutically effective amount of the antibody or an antigen-binding fragment thereof of claim 1, or of the polynucleotide encoding the antibody or antigen-binding fragment thereof of claim 1, or the pharmaceutical composition of claim 30.

37. The method of claim 36, wherein the disease associated with a suppressed immune system is cancer or an infectious disease.

38-44. (canceled)

45. The method of claim 36, wherein the subject is human.

46-49. (canceled)

50. The antibody or antigen-binding fragment thereof of claim 1, comprising a VH and VL, wherein
(a) the VH comprises an amino acid sequence of SEQ ID NO: 15 and the VL comprises an amino acid sequence of SEQ ID NO: 16;
(b) the VH comprises an amino acid sequence of SEQ ID NO: 23 and the VL comprises an amino acid sequence of SEQ ID NO: 24;
(c) the VH comprises an amino acid sequence of SEQ ID NO: 45 and the VL comprises an amino acid sequence of SEQ ID NO: 46;
(d) the VH comprises an amino acid sequence of SEQ ID NO: 92 and the VL comprises an amino acid sequence of SEQ ID NO: 93;
(e) the VH comprises an amino acid sequence of SEQ ID NO: 95 and the VL comprises an amino acid sequence of SEQ ID NO: 96;
(f) the VH comprises an amino acid sequence of SEQ ID NO: 99 and the VL comprises an amino acid sequence of SEQ ID NO: 100;
(g) the VH comprises an amino acid sequence of SEQ ID NO: 101 and the VL comprises an amino acid sequence of SEQ ID NO: 102;
(h) the VH comprises an amino acid sequence of SEQ ID NO: 103 and the VL comprises an amino acid sequence of SEQ ID NO: 104;
(i) the VH comprises an amino acid sequence of SEQ ID NO: 105 and the VL comprises an amino acid sequence of SEQ ID NO: 104;
(j) the VH comprises an amino acid sequence of SEQ ID NO: 105 and the VL comprises an amino acid sequence of SEQ ID NO: 107;
(k) the VH comprises an amino acid sequence of SEQ ID NO: 110 and the VL comprises an amino acid sequence of SEQ ID NO: 111;
(l) the VH comprises an amino acid sequence of SEQ ID NO: 110 and the VL comprises an amino acid sequence of SEQ ID NO: 112;
(m) the VH comprises an amino acid sequence of SEQ ID NO: 110 and the VL comprises an amino acid sequence of SEQ ID NO: 113;
(n) the VH comprises an amino acid sequence of SEQ ID NO: 114 and the VL comprises an amino acid sequence of SEQ ID NO: 111;
(o) the VH comprises an amino acid sequence of SEQ ID NO: 114 and the VL comprises an amino acid sequence of SEQ ID NO: 112;
(p) the VH comprises an amino acid sequence of SEQ ID NO: 114 and the VL comprises an amino acid sequence of SEQ ID NO: 113;
(q) the VH comprises an amino acid sequence of SEQ ID NO: 115 and the VL comprises an amino acid sequence of SEQ ID NO: 111;
(r) the VH comprises an amino acid sequence of SEQ ID NO: 115 and the VL comprises an amino acid sequence of SEQ ID NO: 112;
(s) the VH comprises an amino acid sequence of SEQ ID NO: 115 and the VL comprises an amino acid sequence of SEQ ID NO: 113;
(t) the VH comprises an amino acid sequence of SEQ ID NO: 116 and the VL comprises an amino acid sequence of SEQ ID NO: 111;
(u) the VH comprises an amino acid sequence of SEQ ID NO: 116 and the VL comprises an amino acid sequence of SEQ ID NO: 112;
(v) the VH comprises an amino acid sequence of SEQ ID NO: 116 and the VL comprises an amino acid sequence of SEQ ID NO: 113;
(w) the VH comprises an amino acid sequence of SEQ ID NO: 7 and the VL comprises an amino acid sequence of SEQ ID NO: 8;
(x) the VH comprises an amino acid sequence of SEQ ID NO: 30 and the VL comprises an amino acid sequence of SEQ ID NO: 31;
(v) the VH comprises an amino acid sequence of SEQ ID NO: 38 and the VL comprises an amino acid sequence of SEQ ID NO: 39;
(z) the VH comprises an amino acid sequence of SEQ ID NO: 52 and the VL comprises an amino acid sequence of SEQ ID NO: 53;
(aa) the VH comprises an amino acid sequence of SEQ ID NO: 59 and the VL comprises an amino acid sequence of SEQ ID NO: 60;
(bb) the VH comprises an amino acid sequence of SEQ ID NO: 66 and the VL comprises an amino acid sequence of SEQ ID NO: 67;
(cc) the VH comprises an amino acid sequence of SEQ ID NO: 73 and the VL comprises an amino acid sequence of SEQ ID NO: 74;
(dd) the VH comprises an amino acid sequence of SEQ ID NO: 79 and the VL comprises an amino acid sequence of SEQ ID NO: 80;
(ee) the VH comprises an amino acid sequence of SEQ ID NO: 85 and the VL comprises an amino acid sequence of SEQ ID NO: 86;
(ff) the VH comprises an amino acid sequence of SEQ ID NO: 88 and the VL comprises an amino acid sequence of SEQ ID NO: 89.
